(19) United States
(12) Patent Application Publication
(10) Pub. No.: US 2011/0038885 A1
(43) Pub. Date: Feb. 17, 2011

(54) CHEMOKINE ANALOGS

(76) Inventors: Zheng Xin Dong, Holliston, MA (US); Sun H. Kim, Needham, MA (US)

Correspondence Address: FISH & RICHARDSON P.C. (SV), PO BOX 1022, MINNEAPOLIS, MN, 55440-1022, US

(21) Appl. No.: 12/670,720

(22) PCT Filed: Jul. 21, 2008
(86) PCT No.: PCT/US08/08857

§ 371 Date: Jan. 26, 2010

Related U.S. Application Data
(60) Provisional application No. 60/962,051, filed on Jul. 26, 2007.

(51) Int. Cl. A61K 38/12; C07K 7/64; A61P 37/04; A61P 37/08; A61P 19/02; A61P 37/06; A61P 11/06; A61P 17/06; A61P 35/00; A61P 33/00; A61P 33/06; A61P 19/08; A61P 31/12; A61P 31/18; A61P 31/14; A61P 9/00; A61P 29/00

(52) U.S. Cl. 424/185.1; 530/321; 514/21.1; 514/3.7; 514/3.8; 514/16.7; 514/4.6; 514/19.3; 514/18.7; 514/1.7; 514/16.6

(57) ABSTRACT

The present invention is directed to peptide analogs of chemokine, the pharmaceutically acceptable salts thereof, to methods of using such analogs to treat mammals and to pharmaceutical compositions useful therefor comprising said analogs.

### BACKGROUND OF THE INVENTION

Macrophage and/or monocyte recruitment plays a role in the morbidity and mortality of a broad spectrum of diseases, including autoimmune diseases, granulomatous diseases, infectious diseases, osteoporosis, and coronary artery disease. For example, in atherosclerosis, early during lipid lesion formation, circulating monocytes adhere to the activated endothelium overlying the incipient plaque. Under appropriate conditions, the monocytes then migrate into the developing intima. In the intima, macrophage accumulate lipoprotein and excrete an excess of proteases relative to protease inhibitors. If the lipoproteins are oxidized, they are toxic to macrophage, which results in macrophage death and an increase in an unstable, necrotic, extracellular lipid pool. An excess of proteases results in loss of extracellular matrix and destabilization of the fibrous plaque. Plaque instability, is the acute cause of myocardial infarction.

Many molecules have been identified that are necessary for the recruitment of monocytes and other inflammatory cell types to sites of injury or insult. These molecules represent targets for the inhibition of monocytes recruitment. One class of such molecules is adhesion molecules, e.g., receptors, for monocytes. Another class of molecules includes inflammatory mediators, such as TNFα and related molecules, the interleukins, e.g., IL-1β, and chemokines, e.g., monocytes chemoattractant protein-1 (MCP-1). As a result, agents which modulate the activity of chemokines are likely to be useful to prevent and treat a wide range of diseases. For example, Rollins et al. (U.S. Pat. No. 5,459,128) generally disclose analogs of MCP-1 that inhibit the monocyte chemoattractant activity of endogenous MCP-1. Analogs that are effective to inhibit endogenous MCP-1 are disclosed as analogs which are modified at 28-tyrosine, 24-arginine, 3-aspartate and/or in amino acids between residues 2-8 of MCP-1. In particular, Rollins et al. state that “[s]uccessful inhibition of the activity is found where MCP-1 is modified in one or more of the following ways: a) the 28-tyrosine is substituted by aspartate, b) the 24-arginine is substituted by phenylalanine, c) the 3-aspartate is substituted by alanine, and/or d) the 2-8 amino acid sequence is deleted” (col. 1, lines 49-54). The deletion of amino acids 2-8 of MCP-1 (“MCP-1(Δ2-8)”) results in a polypeptide that is inactive, i.e., MCP-1(Δ2-8) is not a chemoattractant (col. 5, lines 22-23).

Recent studies suggest that MCP-1(Δ2-8) exhibits a dominant negative effect, i.e., it forms heterodimers with wild-type MCP-1 that cannot elicit a biological effect (Zhang et al., Mol. Cell. Biol., 15:4851, 1995). Thus, MCP-1(Δ2-8) does not exhibit properties of a classic receptor antagonist. Moreover, MCP-1(Δ2-8) is unlikely to be widely useful for inhibition of MCP-1 activity in vivo, as MCP-1 (Δ2-8) is a large polypeptide with undesirable pharmacodynamic properties. Furthermore, it is unknown whether MCP-1(Δ2-8) is active as dominant-negative inhibitor of other chemokines associated with inflammation.

Therefore, there is a need to identify agents that inhibit or enhance chemokine-induced macrophage and/or monocyte recruitment and which have desirable pharmacodynamic properties. Moreover, there is a need to identify agents that inhibit or enhance chemokine-induced activities of other cell types, such as lymphocytes, neutrophils or eosinophils. Further, there is a need to identify agents that are pan-selective chemokine inhibitors.

### SUMMARY OF THE INVENTION

The present invention provides a therapeutic agent comprising an isolated and purified chemokine peptide, chemokine peptide variant, chemokine analog, or a derivative thereof. Preferably, the therapeutic agent of the invention inhibits the activity of more than one chemokine, although the agent may not inhibit the activity of all chemokines to the same extent. Alternatively, a preferred therapeutic agent of the invention specifically inhibits the activity of one chemokine to a greater extent than other chemokines. Yet another preferred therapeutic agent of the invention mimics the activity of a chemokine, e.g., it acts as an agonist. Thus, therapeutic agents that are chemokine antagonists and agonists are within the scope of the invention. A further preferred therapeutic agent of the invention is an agent that does not inhibit or mimic the activity of a chemokine but binds to or near the receptor for that chemokine, i.e., it is a neutral agent.

In one aspect, the invention features a peptide of the formula (I):

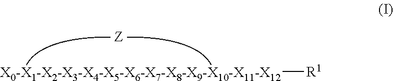

wherein

X₀ is H, pGlu, D-pGlu, (R²,R³)Y₀-Y₁-Y₂-Y₃-Y₄-Y₅-Y₆-Y₇-, or deleted;

X₁ is Cys, hCys, Pen, Lys, Orn, Dab, Dap, Glu, Asp, D-Cys, D-hCys, D-Pen, D-Lys, D-Orn, D-Dab, D-Dap, D-Glu, D-Asp —HN—(CR⁵R⁶)ₙ—C(O)—, —C(O)—(CR⁷R⁸)ₘ—(O)ₓ—(CR⁹C¹⁰)ₛ—C(O)—, or —CH₂—(CR¹¹C¹²)ₜ—C(O)—;

X₂ is Leu, hLeu, Ile, Cha, Val, Abu, D-Leu, D-hLeu, D-Ile, D-Cha, D-Val, D-Abu, or deleted;

X₃ is Aps, Glu, D-Asp, D-Glu, or deleted;

X₄ is Pro, Dmt, hPro, Thz, 3Hyp, 4Hyp, D-Pro, D-Dmt, D-hPro, D-Thi, D-Thz, D-3Hyp, D-4Hyp, or deleted;

X₅ is Lys, Arg, hArg, Orn, Dab, Dap, D-Lys, D-Arg, D-hArg, D-Orn, D-Dab, D-Dap, or deleted;

X₆ is Gln, Asn, D-Gln, D-Asn, or deleted;

X₇ is Lys, Arg, hArg, Orn, Dab, Dap, D-Lys, D-Arg, D-hArg, D-Orn, D-Dab, D-Dap, or deleted;

X₈ is Tip, 1Nal, 2Nal, D-Trp, D-1Nal, D-2Nal, or deleted;

X₉ is Ile, Leu, hLeu, Cha, Val, Abu, D-Ile, D-Leu, D-hLeu, D-Cha, D-Val, or D-Abu;

X₁₀ is Gln, Asn, D-Gln, or D-Asn;

X₁₁ is Cys, hCys, Pen, Lys, Orn, Dab, Dap, Glu, Asp, D-Cys, D-hCys, D-Pen, D-Lys, D-Orn, D-Dab, D-Dap, D-Glu, or D-Asp;

X₁₂ is Lys, Arg, hArg, Orn, Dab, Dap, D-Lys, D-Arg, D-hArg, D-Orn, D-Dab, D-Dap, or deleted;

Y₀ is Aps, Glu, D-Asp, D-Glu, or deleted;

Y₁ is Leu, hLeu, Ile, Cha, Val, Abu, D-Leu, D-hLeu, D-Ile, D-Cha, D-Val, D-Abu, or deleted;

Y₂ is Aps, Glu, D-Asp, D-Glu, or deleted;

Y₃ is Pro, Dmt, hPro, Thz, 3Hyp, 4Hyp, D-Pro, D-Dmt, D-hPro, D-Thi, D-Thz, D-3Hyp, D-4Hyp, or deleted;

Y₄ is Lys, Arg, hArg, Orn, Dab, Dap, D-Lys, D-Arg, D-hArg, D-Orn, D-Dab, D-Dap, or deleted;

Y₅ is Gln, Asn, D-Gln, D-Asn, or deleted;

Y₆ is Lys, Arg, hArg, Orn, Dab, Dap, D-Lys, D-Arg, D-hArg, D-Orn, D-Dab, D-Dap, or deleted;

Y₇ is Tip, 1Nal, 2Nal, D-Trp, D-1Nal, D-2Nal, or deleted;

R¹ is —OH or —NH₂;

provided that:

when X₁ and X₁₁ each independently is Cys, hCys, Pen, D-Cys, D-hCys, or D-Pen, Z is a single covalent bond between —S— in the side-chain of X₁ and —S— in the side-chain of X₁₁;

when X₁ is Lys; Orn, Dab, Dap, D-Lys, D-Orn, D-Dab, D-Dap, or —HN—(CR⁵R⁶)ₙ—C(O)— and X₁₁ is Glu, Aps, D-Glu, or D-Aps, Z is an amide bond;
when X₁ is Glu, Asp, D-Glu, D-Asp, or —C(O)—(CR⁷R⁸)ₘ—(O)ₓ—(CR⁹C¹⁰)ₛ—C(O)—, and X₁₁ is Lys, Orn, Dab, Dap, D-Lys, D-Orn, D-Dab, or D-Dap, Z is an amide bond; and

when X₁ is —CH₂—(CR¹¹C¹²)ₜ—C(O)— and X₁₁ is Cys, hCys, Pen, D-Cys, D-hCys, or D-Pen, Z is a single covalent bond between —CH₂— in the side chain of X₁ and —S— of X₁₁;

further provided that:

X₂ and Y₁ do not coexist;

X₃ and Y₂ do not coexist;

X₄ and Y₃ do not coexist;

X₅ and Y₄ do not coexist;

X₆ and Y₅ do not coexist;

X₇ and Y₆ do not coexist;

X₈ and Y₇ do not coexist; and

one of X₈ and Y₇ is not deleted;

each of R² and R³ is, independently for each occurrence, H, (C₁-C₃₀)alkyl, (C₁-C₃₀)acyl, substituted (C₁-C₃₀)alkyl, substituted (C₁-C₃₀)acyl, arylacyl, or arylakylacyl;

further provided that:

when R² is (C₁-C₃₀)acyl, substituted (C₁-C₃₀)acyl, arylacyl, or arylakylacyl, then R³ is H, (C₁-C₃₀)alkyl, or substituted (C₁-C₃₀)alkyl;

each of R⁵, R⁶, R⁷, R⁸, R⁹, R¹⁰, R¹¹ and R¹² is, independently for each occurrence, H, halogen, or (C₁-C₁₀)alkyl;

R⁵ and R⁶ can joint together to form a ring system;

R⁷ and R⁸ can joint together to form a ring system;

R⁹ and R¹⁰ can joint together to form a ring system;

R¹¹ and R¹² can joint together to form a ring system;

n is 1-20;

m is 1-5;

s is 1-5;

x is 0 or 1; and

t is 1-5.

A more preferred compound of formula (I) is where said compound is of the formula:

(SEQ ID NO: 1)
Ac-c(Cys-Leu-Asp-Pro-Lys-Gln-Lys-Trp-Ile-Gln-Cys)-
NH₂,
(SEQ ID NO: 2)
Leu-c(Cys-Asp-Pro-Lys-Gln-Lys-Trp-Ile-Gln-Cys)-
NH₂,
(SEQ ID NO: 3)
Leu-Asp-c(Cys-Pro-Lys-Gln-Lys-Trp-Ile-Gln-Cys)-
NH₂,
(SEQ ID NO: 4)
Leu-Asp-Pro-Lys-Gln-c(Cys-Lys-Trp-Ile-Gln-Cys)-
NH₂,
(SEQ ID NO: 5)
Leu-Asp-Pro-Lys-Gln-Lys-c(Cys-Trp-Ile-Gln-Cys)-
NH₂,
(SEQ ID NO: 6)
Leu-Asp-Pro-Lys-Gln-Lys-Trp-c(Cys-Ile-Gln-Cys)-
NH₂,
(SEQ ID NO: 7)
Lys-Trp-c(Cys-Ile-Gln-Cys)-NH₂,
(SEQ ID NO: 8)
Ac-c(Cys-Leu-Asp-Pro-Lys-Gln-Lys-Trp-Ile-Gln-Cys)-
D-Dap-NH₂,
(SEQ ID NO: 9)
Ac-Asp-c(Cys-Leu-Asp-Pro-Lys-Gln-Lys-Trp-Ile-Gln-
Cys)-D-Dap-NH₂,
(SEQ ID NO: 8)
Ac-c(Cys-Leu-Asp-Pro-Lys-Gln-Lys-Trp-Ile-Gln-Cys)-
Dab-NH₂,
(SEQ ID NO: 2)
Ac-Leu-c(Cys-Asp-Pro-Lys-Gln-Lys-Trp-Ile-Gln-Cys)-
NH₂,
(SEQ ID NO: 3)
Ac-Leu-Asp-c(Cys-Pro-Lys-Gln-Lys-Trp-Ile-Gln-Cys)-
NH₂,
(SEQ ID NO: 10)
Ac-Leu-Asp-Pro-c(Cys-Lys-Gln-Lys-Trp-Ile-Gln-Cys)-
NH₂,
(SEQ ID NO: 11)
Ac-Leu-Asp-Pro-Lys-c(Cys-Gln-Lys-Trp-Ile-Gln-Cys)-
NH₂,
(SEQ ID NO: 4)
Ac-Leu-Asp-Pro-Lys-Gln-c(Cys-Lys-Trp-Ile-Gln-Cys)-
NH₂,
(SEQ ID NO: 12)
Ac-c(Cys-Leu-Asp-Pro-Lys-Gln-Lys-Trp-Ile-D-Gln-
Cys)-NH₂,
(SEQ ID NO: 13)
Ac-c(Cys-Leu-Asp-Pro-Lys-Gln-Lys-Trp-D-Ile-Gln-
Cys)-NH₂,
(SEQ ID NO: 14)
Ac-c(Cys-Leu-Asp-Pro-Lys-Gln-Lys-D-Trp-Ile-Gln-
Cys)-NH₂,
(SEQ ID NO: 15)
Ac-c(Cys-Leu-Asp-Pro-Lys-Gln-D-Lys-Trp-Ile-Gln-
Cys)-NH₂,
(SEQ ID NO: 16)
Ac-c(Cys-Leu-Asp-Pro-Lys-D-Gln-Lys-Trp-Ile-Gln-
Cys)-NH₂,
(SEQ ID NO: 17)
Ac-c(Cys-Leu-Asp-Pro-D-Lys-Gln-Lys-Trp-Ile-Gln-
Cys)-NH₂,
(SEQ ID NO: 18)
Ac-c(Cys-Leu-Asp-D-Pro-Lys-Gln-Lys-Trp-Ile-Gln-
Cys)-NH₂,
(SEQ ID NO: 19)
Ac-c(Cys-Leu-D-Asp-Pro-Lys-Gln-Lys-Trp-Ile-Gln-
Cys)-NH₂,
(SEQ ID NO: 20)
Ac-c(Cys-D-Leu-Asp-Pro-Lys-Gln-Lys-Trp-Ile-Gln-
Cys)-NH₂,
(SEQ ID NO: 1)
c(Cys-Leu-Asp-Pro-Lys-Gln-Lys-Trp-Ile-Gln-Cys)-
NH₂,
(SEQ ID NO: 21)
Ac-Lys-c(Cys-Leu-Asp-Pro-Lys-Gln-Lys-Trp-Ile-Gln-
Cys)-NH₂,
(SEQ ID NO: 22)
c(D-Cys-Leu-Asp-Pro-Lys-Gln-Lys-Trp-Ile-Gln-Cys)-
Dap-NH₂,
(SEQ ID NO: 23)
c(Cys-Leu-Asp-Pro-Lys-Gln-Lys-Trp-Ile-Gln-D-Cys)-
NH₂,
(SEQ ID NO: 24)
c(D-Cys-Leu-Asp-Pro-Lys-Gln-Lys-Trp-Ile-Gln-D-
Cys)-NH₂,
(SEQ ID NO: 10)
Leu-Asp-Pro-c(Cys-Lys-Gln-Lys-Trp-Ile-Gln-Cys)-
NH₂,
(SEQ ID NO: 25)
c(D-Cys-Leu-Asp-Pro-Lys-Gln-Lys-Trp-Ile-Gln-Cys)-
NH₂,
(SEQ ID NO: 26)
Asp-Pro-c(Cys-Lys-Gln-Lys-Trp-Ile-Gln-Cys)-NH₂,
(SEQ ID NO: 27)
Asp-Pro-Lys-c(Cys-Gln-Lys-Trp-Ile-Gln-Cys)-NH₂,
(SEQ ID NO: 28)
c(Cys-Asp-Pro-Lys-Gln-Lys-Trp-Ile-Gln-Cys)-NH₂,
(SEQ ID NO: 29)
Pro-c(Cys-Lys-Gln-Lys-Trp-Ile-Gln-Cys)-NH₂,
(SEQ ID NO: 30)
Pro-Lys-c(Cys-Gln-Lys-Trp-Ile-Gln-Cys)-NH₂,
(SEQ ID NO: 31)
Pro-Lys-Gln-c(Cys-Lys-Trp-Ile-Gln-Cys)-NH₂,
(SEQ ID NO: 32)
c(Cys-Pro-Lys-Gln-Lys-Trp-Ile-Gln-Cys)-NH₂,
(SEQ ID NO: 33)
Lys-c(Cys-Gln-Lys-Trp-Ile-Gln-Cys)-NH₂,
(SEQ ID NO: 34)
Lys-Gln-c(Cys-Lys-Trp-Ile-Gln-Cys)-NH₂,
(SEQ ID NO: 35)
c(Cys-Lys-Gln-Lys-Trp-Ile-Gln-Cys)-NH₂,
(SEQ ID NO: 36)
c(Cys-Gln-Lys-Trp-Ile-Gln-Cys)-NH₂,
(SEQ ID NO: 37)
Lys-c(Cys-Trp-Ile-Gln-Cys)-NH₂,
(SEQ ID NO: 38)
c(Cys-Lys-Trp-Ile-Gln-Cys)-NH₂,
(SEQ ID NO: 39)
pGlu-c(Cys-Lys-Trp-Ile-Gln-Cys)-NH₂,
(SEQ ID NO: 39)
D-pGlu-c(Cys-Lys-Trp-Ile-Gln-Cys)-NH₂,
(SEQ ID NO: 39)
Ac-Gln-c(Cys-Lys-Trp-Ile-Gln-Cys)-NH₂,
(SEQ ID NO: 39)
Ac-D-Gln-c(Cys-Lys-Trp-Ile-Gln-Cys)-NH₂,
(SEQ ID NO: 38)
Ac-c(Cys-Lys-Trp-Ile-Gln-Cys)-NH₂,
(SEQ ID NO: 38)
nButyryl-c(Cys-Lys-Trp-Ile-Gln-Cys)-NH₂,
(SEQ ID NO: 38)
Benzoyl-c(Cys-Lys-Trp-Ile-Gln-Cys)-NH₂,
(SEQ ID NO: 38)
Isobutyryl-c(Cys-Lys-Trp-Ile-Gln-Cys)-NH₂,
(SEQ ID NO: 40)
c(βAla-Lys-Trp-Gln-Asp)-NH₂,
(SEQ ID NO: 41)
c(Gaba-Lys-Trp-Ile-Gln-Asp)-NH₂,
(SEQ ID NO: 42)
c(Gaba-Lys-Trp-Ile-Gln-Glu)-NH₂,
(SEQ ID NO: 43)
c(Succinyl-Lys-Trp-Ile-Gln-Orn)-NH₂,
(SEQ ID NO: 44)
c(Succinyl-Lys-Trp-Ile-Gln-Lys)-NH₂,
(SEQ ID NO: 45)
Ac-c(Asp-Lys-Trp-Ile-Gln-Lys)-NH₂,
(SEQ ID NO: 46)
c(Ac-Lys-Trp-Ile-Gln-Cys)-NH₂,
(SEQ ID NO: 38)
Trimethylacetyl-c(Cys-Lys-Trp-Ile-Gln-Cys)-NH₂,
(SEQ ID NO: 38)
4-Biphenylcarboxyl-c(Cys-Lys-Trp-Ile-Gln-Cys)-NH₂,
(SEQ ID NO: 38)
1-Naphthoyl-c(Cys-Lys-Trp-Ile-Gln-Cys)-NH₂,
(SEQ ID NO: 38)
2-Naphthoyl-c(Cys-Lys-Trp-Ile-Gln-Cys)-NH₂,
(SEQ ID NO: 38)
2-Indolecarboxyl-c(Cys-Lys-Trp-Ile-Gln-Cys)-NH₂,
(SEQ ID NO: 38)
p-Tolylacetyl-c(Cys-Lys-Trp-Ile-Gln-Cys)-NH₂,
(SEQ ID NO: 38)
trans-Cinnamoyl-c(Cys-Lys-Trp-Ile-Gln-Cys)-NH₂,
(SEQ ID NO: 38)
4-Chlorocinnamoyl-c(Cys-Lys-Trp-Ile-Gln-Cys)-NH₂,
(SEQ ID NO: 47)
c(Cys-Trp-Ile-Gln-Cys)-NH₂,
(SEQ ID NO: 48)
Ac-c(Cys-Lys-Trp-Ile-Gln-D-Cys)-NH₂,
(SEQ ID NO: 49)
Ac-c(Cys-Lys-Trp-Ile-D-Gln-Cys)-NH₂,
(SEQ ID NO: 50)
Ac-c(Cys-Lys-Trp-D-Ile-Gln-Cys)-NH₂,
(SEQ ID NO: 51)
Ac-c(Cys-Lys-D-Trp-Ile-Gln-Cys)-NH₂,
(SEQ ID NO: 52)
Ac-c(Cys-D-Lys-Trp-Ile-Gln-Cys)-NH₂,
(SEQ ID NO: 53)
Ac-c(D-Cys-Lys-Trp-Ile-Gln-Cys)-NH₂,
(SEQ ID NO: 54)
(Des-amino-4-methyl)Phe-c(Cys-Lys-Trp-Ile-Gln-
Cys)-NH₂,
(SEQ ID NO: 38)
n-Hexanoyl-c(Cys-Lys-Trp-Ile-Gln-Cys)-NH₂,
(SEQ ID NO: 38)
Octanoyl-c(Cys-Lys-Trp-Ile-Gln-Cys)-NH₂,
(SEQ ID NO: 38)
Decanoyl-c(Cys-Lys-Trp-Ile-Gln-Cys)-NH₂,
(SEQ ID NO: 38)
Dodecanoyl-c(Cys-Lys-Trp-Ile-Gln-Cys)-NH₂,
(SEQ ID NO: 55)
Trp-c(Cys-Ile-Gln-Cys)-NH₂,
(SEQ ID NO: 56)
c(Cys-Ile-Gln-Cys)-NH₂,
(SEQ ID NO: 57)
c(Ac-Lys-Trp-Ile-Gln-hCys)-NH₂,
(SEQ ID NO: 58)
c(Gly-Lys-Trp-Ile-Gln-Asp)-NH₂,
(SEQ ID NO: 58)
c(Gly-Lys-Trp-Ile-Gln-Glu)-NH₂,
(SEQ ID NO: 42)
c(β3Ala-Lys-Trp-Ile-Gln-Glu)-NH₂,
(SEQ ID NO: 41)
c(Apn-Lys-Trp-Ile-Gln-Asp)-NH₂,
(SEQ ID NO: 42)
c(Apn-Lys-Trp-Ile-Gln-Glu)-NH₂,
(SEQ ID NO: 41)
c(Ahx-Lys-Trp-Ile-Gln-Asp)-NH₂,
(SEQ ID NO: 42)
c(Ahx-Lys-Trp-Ile-Gln-Glu)-NH₂,
(SEQ ID NO: 41)
c(Aha-Lys-Trp-Ile-Gln-Asp)-NH₂,
(SEQ ID NO: 42)
c(Aha-Lys-Trp-Ile-Gln-Glu)-NH₂,
(SEQ ID NO: 41)
c(Aoc-Lys-Trp-Ile-Gln-Asp)-NH₂,
(SEQ ID NO: 42)
c(Aoc-Lys-Trp-Ile-Gln-Glu)-NH₂,
(SEQ ID NO: 41)
c(Anc-Lys-Trp-Ile-Gln-Asp)-NH₂,
(SEQ ID NO: 42)
c(Anc-Lys-Trp-Ile-Gln-Glu)-NH₂,
(SEQ ID NO: 59)
Isobutyryl-c(Asp-Lys-Trp-Ile-Gln-Dap)-NH₂,
(SEQ ID NO: 59)
Isobutyryl-c(Asp-Lys-Trp-Ile-Gln-Orn)-NH₂,
(SEQ ID NO: 60)
Isobutyryl-c(Glu-Lys-Trp-Ile-Gln-Dap)-NH₂,
(SEQ ID NO: 60)
Isobutyryl-c(Glu-Lys-Trp-Ile-Gln-Dab)-NH₂,
(SEQ ID NO: 60)
Isobutyryl-c(Glu-Lys-Trp-Ile-Gln-Orn)-NH₂,
(SEQ ID NO: 60)
Isobutyryl-c(Glu-Lys-Trp-Ile-Gln-Lys)-NH₂,
(SEQ ID NO: 61)
Isobutyryl-c(Dap-Lys-Trp-Ile-Gln-Asp)-NH₂,
(SEQ ID NO: 61)
Isobutyryl-c(Dab-Lys-Trp-Ile-Gln-Asp)-NH₂,
(SEQ ID NO: 61)
Isobutyryl-c(Lys-Lys-Trp-Ile-Gln-Asp)-NH₂,
(SEQ ID NO: 62)
Isobutyryl-c(Dap-Lys-Trp-Ile-Gln-Glu)-NH₂,
(SEQ ID NO: 62)
Isobutyryl-c(Dab-Lys-Trp-Ile-Gln-Glu)-NH₂,
(SEQ ID NO: 62)
Isobutyryl-c(Orn-Lys-Trp-Ile-Gln-Glu)-NH₂,
(SEQ ID NO: 62)
Isobutyryl-c(Lys-Lys-Trp-Ile-Gln-Glu)-NH₂,
(SEQ ID NO: 63)
Ac-c(Asp-Lys-Trp-Ile-Gln-Dap)-NH₂,
(SEQ ID NO: 63)
Ac-c(Asp-Lys-Trp-Ile-Gln-Dab)-NH₂,
(SEQ ID NO: 63)
Ac-c(Asp-Lys-Trp-Ile-Gln-Orn)-NH₂,
(SEQ ID NO: 64)
Ac-c(Glu-Lys-Trp-Ile-Gln-Dap)-NH₂,
(SEQ ID NO: 64)
Ac-c(Glu-Lys-Trp-Ile-Gln-Dab)-NH₂,
(SEQ ID NO: 64)
Ac-c(Glu-Lys-Trp-Ile-Gln-Orn)-NH₂,
(SEQ ID NO: 64)
Ac-c(Glu-Lys-Trp-Ile-Gln-Lys)-NH₂,
(SEQ ID NO: 65)
Ac-c(Dap-Lys-Trp-Ile-Gln-Asp)-NH₂,
(SEQ ID NO: 65)
Ac-c(Dab-Lys-Trp-Ile-Gln-Asp)-NH₂,
(SEQ ID NO: 65)
Ac-c(Orn-Lys-Trp-Ile-Gln-Asp)-NH₂,
(SEQ ID NO: 65)
Ac-c(Lys-Lys-Trp-Ile-Gln-Asp)-NH₂,
(SEQ ID NO: 66)
Ac-c(Dap-Lys-Trp-Ile-Gln-Glu)-NH₂,
(SEQ ID NO: 66)
Ac-c(Dab-Lys-Trp-Ile-Gln-Glu)-NH₂,
(SEQ ID NO: 66)
Ac-c(Orn-Lys-Trp-Ile-Gln-Glu)-NH₂,
(SEQ ID NO: 66)
Ac-c(Lys-Lys-Trp-Ile-Gln-Glu)-NH₂,
(SEQ ID NO: 43)
c(Succinyl-Lys-Trp-Ile-Gln-Dap)-NH₂,
(SEQ ID NO: 43)
c(Succinyl-Lys-Trp-Ile-Gln-Dab)-NH₂,
(SEQ ID NO: 67)
c(Glutaryl-Lys-Trp-Ile-Gln-Dap)-NH₂,
(SEQ ID NO: 67)
c(Glutaryl-Lys-Trp-Ile-Gln-Dab)-NH₂,
(SEQ ID NO: 67)
c(Glutaryl-Lys-Trp-Ile-Gln-Orn)-NH₂,
(SEQ ID NO: 67)
c(Glutaryl-Lys-Trp-Ile-Gln-Lys)-NH₂,
(SEQ ID NO: 68)
c(Diglycolyl-Lys-Trp-Ile-Gln-Dap)-NH₂,
(SEQ ID NO: 68)
c(Diglycolyl-Lys-Trp-Ile-Gln-Dab)-NH₂,
(SEQ ID NO: 68)
c(Diglycolyl-Lys-Trp-Ile-Gln-Orn)-NH₂,
(SEQ ID NO: 68)
c(Diglycolyl-Lys-Trp-Ile-Gln-Lys)-NH₂,
(SEQ ID NO: 69)
c(Adipoyl-Lys-Trp-Ile-Gln-Dap)-NH₂,
(SEQ ID NO: 69)
c(Adipoyl-Lys-Trp-Ile-Gln-Dab)-NH₂,
(SEQ ID NO: 69)
c(Adipoyl-Lys-Trp-Ile-Gln-Orn)-NH₂,
or
(SEQ ID NO: 69)
c(Adipoyl-Lys-Trp-Ile-Gln-Lys)-NH₂,

or a pharmaceutically acceptable salt thereof.

More preferred of the immediately foregoing group of compounds is a compound of the formula:

(SEQ ID NO: 38)
c(Cys-Lys-Trp-Ile-Gln-Cys)-NH₂,
(SEQ ID NO: 39)
Ac-Gln-c(Cys-Lys-Trp-Ile-Gln-Cys)-NH₂,
(SEQ ID NO: 38)
Isobutyryl-c(Cys-Lys-Trp-Ile-Gln-Cys)-NH₂,
(SEQ ID NO: 46)
c(Ac-Lys-Trp-Ile-Gln-Cys)-NH₂,
(SEQ ID NO: 38)
2-Indolecarboxyl-c(Cys-Lys-Trp-Ile-Gln-Cys)-NH₂,
(SEQ ID NO: 47)
c(Cys-Trp-Ile-Gln-Cys)-NH₂,
(SEQ ID NO: 38)
n-Hexanoyl-c(Cys-Lys-Trp-Ile-Gln-Cys)-NH₂,
(SEQ ID NO: 58)
c(Gly-Lys-Trp-Ile-Gln-Glu)-NH₂,
(SEQ ID NO: 41)
c(Ahx-Lys-Trp-Ile-Gln-Asp)-NH₂,
(SEQ ID NO: 42)
c(Ahx-Lys-Trp-Ile-Gln-Glu)-NH₂,
(SEQ ID NO: 42)
c(Aoc-Lys-Trp-Ile-Gln-Glu)-NH₂,
(SEQ ID NO: 59)
Isobutyryl-c(Asp-Lys-Trp-Ile-Gln-Dap)-NH₂,
(SEQ ID NO: 60)
Isobutyryl-c(Glu-Lys-Trp-Ile-Gln-Dab)-NH₂,
or
(SEQ ID NO: 62)
Isobutyryl-c(Dap-Lys-Trp-Ile-Gln-Glu)-NH₂,

or a pharmaceutically acceptable salt thereof.

Still more preferred of the immediately foregoing group of compounds is a compound of the formula:

| | |
|---|---|
| c(Cys-Trp-Ile-Gln-Cys)-NH₂, | (SEQ ID NO: 47) |
| or | |
| c(Gly-Lys-Trp-Ile-Gln-Glu)-NH₂, | (SEQ ID NO: 58) |

or a pharmaceutically acceptable salt thereof.

The invention also features peptides of the following formulae:

(SEQ ID NO: 70)
c(D-Cys-D-Gln-D-Ile-DTrp-D-Lys-D-Gln-D-Lys-D-Pro-
D-Asp-D-Leu-D-Cys)-Doc-Aoc-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Aoc-OH,
(SEQ ID NO: 72)
c(D-Cys-D-Gln-D-Ile-D-Trp-D-Lys-D-Gln-D-Lys-D-Pro-
D-Asp-D-Leu-D-Cys)-Ile-Glu-Aoc-OH,
(SEQ ID NO: 73)
c(D-Cys-D-Gln-D-Ile-D-Trp-D-Lys-D-Gln-D-Lys-D-Pro-
D-Asp-D-Leu-D-Cys)-D-Val-D-Glu-Aoc-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Gaba-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Ahx-OH,
(SEQ ID NO: 70)
c(D-Cys-D-Gln-D-Ile-D-Trp-D-Lys-D-Gln-D-Lys-D-Pro-
D-Asp-D-Leu-D-Cys)-Asp-Aoc-OH,
(SEQ ID NO: 70)
c(D-Cys-D-Gln-D-Ile-D-Trp-D-Lys-D-Gln-D-Lys-D-Pro-
D-Asp-D-Leu-D-Cys)-Glu-Aoc-OH,
(SEQ ID NO: 74)
c(D-Cys-D-Gln-D-Ile-D-Trp-D-Lys-D-Gln-D-Lys-D-Pro-
D-Asp-D-Leu-D-Cys)-D-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-D-Asp-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-Asp-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-Lys(Nε-octanoyl)-OH,
(SEQ ID NO: 76)
c(D-Cys-D-Gln-D-Ile-D-Trp-D-Lys-D-Gln-D-Lys-D-Pro-
D-Asp-D-Leu-D-Cys)-D-Val-D-Glu-Lys(Nε-octanoyl)-
0H,
(SEQ ID NO: 75)
c(D-Cys-D-Gln-D-Ile-D-Trp-D-Lys-D-Gln-D-Lys-D-Pro-
D-Asp-D-Leu-D-Cys)-Doc-Lys(Nε-octanoyl)-0H,
(SEQ ID NO: 77)
HEPA-c(D-Cys-D-Gln-D-Ile-D-Trp-D-Lys-D-Gln-D-Lys-
D-Pro-D-Asp-D-Leu-D-Cys)-NH₂,
(SEQ ID NO: 78)
c(D-Cys-D-Gln-D-Ile-D-Trp-D-Lys-D-Gln-D-Lys-D-Pro-
D-Asp-D-Leu-Lys(Nε-octanoyl)-D-Cys)-NH₂,
(SEQ ID NO: 79)
c(D-Cys-D-Gln-D-Ile-D-Trp-D-Lys-D-Gln-D-Lys-D-Pro-
D-Asp-Lys(Nε-octanoyl)-D-Leu-D-Cys)-OH,
(SEQ ID NO: 13)
Ac-c(Cys-Leu-Asp-Pro-Lys-Gln-Lys-Trp-A6c-Gln-Cys)-
NH₂,
(SEQ ID NO: 80)
Ac-c(Cys-A6c-Asp-Pro-Lys-Gln-Lys-Trp-Gln-Cys)-NH₂,
(SEQ ID NO: 81)
Ac-c(Cys-Lys-Gln-Lys-Trp-Ile-Gln-Cys)-D-Pro-D-Asp-
D-Leu-NH₂,
(SEQ ID NO: 82)
Ac-c(Cys-Pro-Lys-Gln-Lys-Trp-Ile-Gln-Cys)-D-Asp-D-
Leu-NH₂,
(SEQ ID NO: 83)
c(D-Cys-D-Gln-D-Ile-D-Trp-D-Lys-D-Gln-D-Lys-D-Pro-
D-Asp(D-Ala-OH)-D-Leu-D-Cys)-NH₂,
(SEQ ID NO: 84)
Octanoyl-Dap-c(D-Cys-D-Gln-D-Ile-D-Trp-D-Lys-D-
Gln-D-Lys-D-Pro-D-Asp-D-Leu-D-Cys)-NH₂,
(SEQ ID NO: 111)
c(D-Cys-D-Gln-D-Ile-D-Trp-D-Lys-D-Gln-D-Lys-D-Pro-
D-Asp(1-hexanol)-D-Leu-D-Cys)-NH₂,
(SEQ ID NO: 85)
Ac-Lys(Nε-dodecanoyl)-c(Cys-Leu-Asp-Pro-Lys-Gln-
Lys-Trp-Ile-Gln-Cys)-NH₂,
(SEQ ID NO: 86)
BOC-Trp-Gly-α-aminoglutaramide,
(SEQ ID NO: 86)
BOC-Trp-Val-α-aminoglutaramide,
(SEQ ID NO: 86)
BOC-Trp-βAla-α-aminoglutaramide,
(SEQ ID NO: 87)
D-Lys(Nε-tetradecanoyl)-D-Asp-c(Cys-Leu-Asp-
Pro-Lys-Gln-Lys-Trp-Ile-Gln-Cys)-NH₂,
(SEQ ID NO: 88)
D-Lys-D-Asp-c(Cys-Leu-Asp-Pro-Lys-Gln-Lys-Trp-Ile-
Gln-Cys)-NH₂,
(SEQ ID NO: 87)
D-Lys(Nε-octanoyl)-D-Asp-c(Cys-Leu-Asp-Pro-Lys-
Gln-Lys-Trp-Ile-Gln-Cys)-NH₂,
(SEQ ID NO: 89)
Lys(Nε-dodecanoyl)-Ile-c(Cys-Leu-Asp-Pro-Lys-Gln-
Lys-Trp-Ile-Gln-Cys)-NH₂,
(SEQ ID NO: 89)
Lys(Nε-dodecanoyl)-Asp-c(Cys-Leu-Asp-Pro-Lys-Gln-
Lys-Trp-Ile-Gln-Cys)-NH₂,
(SEQ ID NO: 90)
BOC-Tcc-βA1a-alpha-aminoglutaramide,
(SEQ ID NO: 91)
D-Lys(Nε-dodecanoyl)-Asp-c(Cys-Leu-Asp-Pro-Lys-
Gln-Lys-Trp-Ile-Gln-Cys)-NH₂,
(SEQ ID NO: 92)
Lys{Nε-[γ-Glu-(Nα-decanoyl)]}-c(Cys-Leu-Asp-Pro-
Lys-Gln-Lys-Trp-Ile-Gln-Cys)-NH₂,
(SEQ ID NO: 90)
BOC-Tic-βAla-a-aminoglutaramide,
(SEQ ID NO: 93)
3-indolepropionyl-L-valyl-α-aminoglutaramide,
(SEQ ID NO: 94)
N(3-indolylpropionyl)-βAla-α-aminoglutarimide,
(SEQ ID NO: 95)
Des-amino-Trp-βAla[ψ(CH₂NH)]-α-aminoglutarimide,
(SEQ ID NO: 94)
N-(3-indolepropyl)-βAla-α-aminoglutarimide,
(SEQ ID NO: 96)
N-(isopropoxycarbonyl)-Trp-βAla-α-
aminoglutarimide,
(SEQ ID NO: 97)
Trp-Gly-α-aminoglutaramide,
(SEQ ID NO: 97)
Trp-Val-α-aminoglutaramide,
(SEQ ID NO: 97)
Trp-βAla-α-aminoglutaramide,
(SEQ ID NO: 98)
c(D-Cys-D-Gln-D-Ile-D-Trp-D-Lys-D-Cys)-NH₂,
(SEQ ID NO: 99)
BOC-Trp-βhVal-α-aminoglutarimide,
(SEQ ID NO: 100)
Tryptaminylcarbonyl-βhVal-α-aminoglutarimide,
(SEQ ID NO: 101)
[aminoPeg₁₀-oxaglutaryl]₂-c(D-Cys-D-Gln-D-Ile-D-
Trp-D-Lys-D-Gln-D-Lys-D-Pro-D-Asp-D-Leu-D-Cys)-
NH₂,
(SEQ ID NO: 101)
[aminoPeg₁₀-oxaglutaryl]-c(D-Cys-D-Gln-D-Ile-D-
Trp-D-Lys-D-Gln-D-Lys-D-Pro-D-Asp-D-Leu-D-Cys)-
NH₂,
(SEQ ID NO: 102)
Trp-Aib-aminoglutarimide,
(SEQ ID NO: 103)
BOC-tryptophanolyl-carbonyl-Val-aminoglutarimide,
(SEQ ID NO: 98)
Ac-c(D-Cys-D-Gln-D-Ile-D-Trp-D-Lys-D-Cys)-NH₂,
(SEQ ID NO: 98)
nButyryl-c(D-Cys-D-Gln-D-Ile-D-Trp-D-Lys-D-Cys)-
NH₂,
(SEQ ID NO: 98)
Benzoyl-c(D-Cys-D-Gln-D-Ile-D-Trp-D-Lys-D-Cys)-
NH₂,
(SEQ ID NO: 98)
Isobutyryl-c(D-Cys-D-Gln-D-Ile-D-Trp-D-Lys-D-Cys)-
NH₂,
(SEQ ID NO: 104)
Amino-tryptophanolyl-carbonyl-Val-
aminoglutarimide,
(SEQ ID NO: 105)
c(βAla-D-Gln-D-Ile-D-Trp-D-Lys-D-Asp)-NH₂,
(SEQ ID NO: 105)
c(Gaba-D-Gln-D-Ile-D-Trp-D-Lys-D-Asp)-NH₂,
(SEQ ID NO: 105)
c(Apn-D-Gln-D-Ile-D-Trp-D-Lys-D-Asp)-NH₂,
(SEQ ID NO: 106)
Ac-c(D-Cys-D-Gln-D-Ile-D-Trp-D-Lys-D-Gln-D-Lys-D-
Pro-D-Asp-D-Leu-D-Cys)-NH₂,
(SEQ ID NO: 107)
c(βAla-D-Gln-D-Ile-D-Trp-D-Lys-D-Gln-D-Lys-D-Pro-
D-Asp-D-Leu-D-Asp)-NH₂,
(SEQ ID NO: 108)
Butanyl-c(D-Cys-D-Gln-D-Val-D-Trp-D-Lys-D-Cys)-
NH₂,
(SEQ ID NO: 109)
Isobutyryl-Asp-Lys-Trp-Ile-Gln-Lys-NH₂,
or
(SEQ ID NO: 110)
Isobutyryl-Orn-Lys-Trp-Ile-Gln-Asp-NH₂,

or a pharmaceutically acceptable salt thereof.

To identify a peptide of the invention useful in the methods of the invention, a sequence comparison of chemokines from different species can be performed. Then the cross-reactivity of a non-human chemokine for the human receptor is assessed. A preferred chemokine is one from a species which has the least sequence homology to the corresponding human chemokine, but which still cross-reacts by binding to the human receptor. Regions which have a high degree of sequence similarity or identity are employed to prepare a peptide of the invention. For example, to identify peptide of TGF-beta having antagonist, agonist or neutral properties, the amino acid sequence of human TGF-beta can be compared to that of Xenopus.

It is envisioned that the therapeutic agents of the invention include compounds having a chiral center that can be isolated in optically active and racemic forms.

Also provided are pharmaceutical compositions, delivery systems, and kits comprising the therapeutic agents of the invention.

The invention further provides methods to treat chemokine-associated indications. For example, the invention provides a method of preventing or inhibiting an indication associated with chemokine-induced activity. The method comprises administering to a mammal afflicted with, or at risk of, the indication an amount of a compound of formula (I) effective to prevent or inhibit said activity. Preferably, the administration is effective to inhibit the activity of more than one chemokine (i.e., the peptide is a pan-selective inhibitor). These agents are useful to treat indications such as acute and chronic inflammatory diseases, a disease with an autoimmune component, psoriasis, rheumatoid arthritis, inflammatory bowel disease, gouty arthritis, brain inflammation, sepsis, septic shock, acute respiratory distress syndrome, hemorrhagic shock, cardiogenic shock, hypovolemic shock, ischemia and reperfusion injury, multiple sclerosis, pulmonary fibrosis, organ transplant rejection, allergy, chronic obstructive pulmonary disease, asthma, and endometriosis.

The invention further provides a method to increase, augment or enhance a chemokine-associated inflammatory response in a mammal, comprising administering to the mammal an amount of a compound of formula (I) effective to increase, augment or enhance said response. These therapeutic agents are useful to increase an inflammatory response to, for example, intracellular pathogens or parasites, which often are associated with a poor immune response. Thus, these agents may be useful to treat or prevent tuberculosis and malaria. Therefore, the invention also provides a therapeutic method to prevent or treat parasitic infection.

The invention also provides a method of preventing or inhibiting an indication associated with histamine release from basophils or mast cells. The method comprises administering to a mammal at risk of, or afflicted with, the indication an effective amount of a compound of formula (I).

Also provided is a method of preventing or inhibiting an indication associated with monocyte, macrophage, neutrophil, B cell, T cell or eosinophil recruitment, or B cell or T cell activation or proliferation. The method comprises administering an effective amount of a compound of formula (I).

Further provided is a therapeutic method to prevent or treat vascular indications, comprising administering to a mammal in need of such therapy an effective amount of a compound of formula (I), wherein the indication is coronary artery disease, myocardial infarction, unstable angina pectoris, atherosclerosis or vasculitis.

The invention also provides a method to prevent or treat an autoimmune disorder. The method comprises administering to a mammal in need of such therapy an effective amount of a compound of formula (I).

Further provided is a method to modulate the chemokine-induced activity of macrophage, B cells, T cells or other hematopoietic cells, e.g., neutrophils, eosinophils or mast cells, at a preselected physiological site. The method comprises administering a dosage comprising an effective amount of a compound of formula (I), wherein the dosage form is linked, either covalently or non-covalently, to a targeting moiety. The targeting moiety binds to a cellular component at the preselected physiological site.

Moreover, it is also envisioned that an agent of the invention may be a targeting moiety, as some of the agents are selective chemokine inhibitors, rather than pan-chemokine inhibitors. For example, an agent of the invention may be useful in the targeted delivery of an isotope or other cytotoxic molecule to red cells for the treatment of disorders such as erythroid leukemia, erythroid myelosis, polycythemia vera or other erythroid dysplasias. Similarly, an agent of the invention that specifically targets a particular cell type may be useful in diagnostics. Thus, these agents can be radiolabeled (Chianelli et al., Nucl. Med. Comm., 18:437, 1997), or labeled with any other detectable signal, such as those useful in diagnostic imaging (e.g., MRI and CAT) to image sites of inflammation in disorders like rheumatoid arthritis and diabetes mellitus (type I).

Also provided is a therapeutic method to augment an immune response. The method comprises administering to a mammal an immunogenic moiety and an amount of a compound of formula (I), wherein the amount is effective to augment the immune response of the mammal to the immunogenic moiety. Thus, the invention also provides a vaccine comprising an immunogenic moiety and an amount of a compound of formula (I). As used herein, an “immunogenic moiety” means an isolated or purified composition or compound (e.g., a purified virus preparation or a native or recombinant viral or bacterial antigen) that, when introduced into an animal, preferably a mammal, results in a humoral and/or cellular immune response by the animal to the composition or compound. It is envisaged that the modified vaccine is delivered by the same routes as those used for unmodified immunogen (e.g., intravenous, intramuscular or orally).

The invention also provides a therapeutic method to prevent or inhibit asthma. The method comprises administering to a mammal in need of such therapy an effective amount of a compound of formula (I). Preferably, in this embodiment of the invention, a therapeutic agent is administered to the upper and/or lower respiratory tract.

Further provided is a therapeutic method to prevent or inhibit viral, e.g., poxvirus, herpesvirus (e.g., Herpesvirus samiri), cytomegalovirus (CMV) or lentivirus, infection or replication. The method comprises administering to a mammal in need of such therapy an effective amount of a compound of formula (I). Preferably, the therapeutic agents are employed to prevent or treat HIV. More preferably, the agent is administered before, during or after the administration of an anti-viral agent, e.g., for HIV AZT, a non-nucleoside reverse transcriptase inhibitor, a protease inhibitor or a combination thereof.

A therapeutic method to prevent or treat low bone mineral density is also provided. The method comprises administering to a mammal in need of such therapy an effective amount of a compound of formula (I).

Also provided is a method of suppressing tumor growth in a vertebrate animal comprising administering to said vertebrate a therapeutically effective amount of a compound of formula (I). Preferably, the method increases or enhances macrophages, B cell-, T cell- or other immune cell-associated activity at a tumor site.

Further provided is a method for preventing or treating rheumatoid arthritis in a mammal, comprising administering to the mammal an effective amount of a compound of formula (I).

Also provided is a method to prevent or treat organ transplant rejection. The method comprises administering an effective amount of a compound of formula (I).

Further provided is a method for preventing or treating psoriasis in a mammal, comprising administering to the mammal an effective amount of a compound of formula (I).

Also provided is a method to enhance wound healing. The method comprises administering an effective amount of a compound of formula (I).

Further provided is a method for preventing or treating an allergy in a mammal, comprising administering to the mammal an effective amount of a compound of formula (I).

Yet another embodiment of the invention is a method to prevent or inhibit an indication associated with elevated TNFα. The method comprises administering an effective amount of a compound of formula (I).

The invention also provides methods whereby the pharmacokinetics of desirable pharmaceutical agents may be modulated. In particular, agents which are normally rapidly cleared from the circulation may be retained longer by the addition of a peptide of the invention that has affinity for the Duffy antigen on red blood cells. This methodology may be particularly suited to modifying the pharmacokinetics of other biologically active, pharmaceutically useful peptides, as well as larger polypeptide or proteins. For example, a Duffy binding peptide may be coupled or linked, either covalently or non-covalently, to a molecule such as recombinant human growth hormone (HGH) or insulin, and administered via a depo injection. By partitioning the modified HGH to the red blood cells, HGH may have much more suitable pharmacokinetics, with longer half lives and less rapid changes in plasma concentrations. In another example, insulin coupled to a peptide of the invention may be particularly useful as a treatment for the highly insulin-resistant type II diabetic whose disease has progressed significantly. It is also envisioned that other small molecules may be coupled to Duffy binding molecules in a manner which preserves the intended function of the active molecule and of the Duffy binding molecule.

### DETAILED DESCRIPTION OF THE INVENTION

One skilled in the art can, based on the description herein, utilize the present invention to its fullest extent. The following specific embodiments are, therefore, to be construed as merely illustrations of the invention and are not meant to be construed as limiting the full scope of the invention in any way.

Abbreviations of the common amino acids are in accordance with the recommendations of IUPAC-IUB. The following are abbreviations of certain amino acids as may appear herein:

Abu α-aminobutyric acid

Aha 7-aminoheptanoic acid

Ahx 6-aminohexanoic acid

Aib α-aminoisobutyric acid

Ala or A alanine

βAla β-alanine

Anc 9-aminononanoic acid

Aoc 8-aminooctanoic acid

Apn 5-aminopentanoic acid

Arg or R arginine

hArg homoarginine

Asn or N asparagine

Asp or D aspartic acid

Cha β-cyclohexylalanine

Cys or C cysteine

hCys homocysteine

Dab 2,4-diaminobutyric acid

Dap 2,3-diaminopropionic acid

Dmt 5,5-dimethylthiazolidine-4-carboxylic acid

Doc 8-amino-3,6-dioxaoctanoic acid

Gaba 4-aminobutyric acid

Gln or Q glutamine

Glu or E glutamic acid

Gly or G glycine

His or H histidine

3Hyp trans-3-hydroxyproline

4Hyp trans-4-hydroxyproline

Ile or I isoleucine

Leu or L leucine

hLeu homoleucine

Lys or K lysine

Met or M methionine

1Nal β-(1-naphthyl)alanine:

2Nal β-(2-naphthyl)alanine

Nle norleucine

Orn ornithine

Pen penicillamine

pGlu pyroglutamic acid

Phe or F phenylalanine

Pro or P proline

hPro homoproline

Ser or S serine

Thr or T threonine

Thz thiaproline

Tcc (S)-2,3,4,5-tetrahydro-B-carboline-4-carboxylic acid

Tic tetrahydroisoquinoline-3-carboxylic acid

Trp or W tryptophan

Tyr or Y tyrosine

Val or V valine

βhVal β-homovaline

Additional abbreviations used herein include:

BOC tert-butyloxycarbonyl

BSA bovine serum albumin

Bzl benzyl

DCM dichloromethane

DIC N,N-diisopropylcarbodiimide

DIEA diisopropylethyl amine

Dmab 4-{N-(1-(4,4-dimethyl-2,6-dioxocyclohexylidene)-3-methylbutyl)-amino}benzyl

DMAP 4-(dimethylamino)pyridine

DMF dimethylformamide

DNP 2,4-dinitrophenyl

FCS fetal calf serum

Fm fluorenylmethyl

Fmoc fluorenylmethyloxycarbonyl

For formyl

HATU O-(7-azabenzotriazolyl)-1,1,3,3-tetramethyluronium hexafluorophosphate

HBTU 2-(1H-benzotriazole-1-yl)-1,1,3,3-tetramethyluronium hexafluorophosphate

HEPA 4-hydroxyethyl-1-carboxymethyl-piperazine

cHex cyclohexyl

HOAT O-(7-azabenzotriazol-1-yl)-1,1,3,3-tetramethyluronium hexafluorophosphate

HOBt 1-hydroxy-benzotriazole

MBHA 4-methylbenzhydrylamine

Mint 4-methoxytrityl

Mtt 4-methyltrityl

NMP N-methylpyrrolidone

Pbf 2,2,4,6,7-pentamethyldihydrobenzofuran-5-sulfonyl

Peg polyethyleneglycol

2-PhiPr 2-phenylisopropyl

PyBop benzotriazole-1-yl-oxy-tris-pyrrolidino-phosphonium hexafluorophosphate

PyBroP bromo-tris-pyrrolidino-phosphonium hexafluorophosphate

tBu tert-butyl

TIS triisopropylsilane

TOS tosyl

Trt trityl

TFA trifluoro acetic acid

TFFH tetramethylfluoroforamidinium hexafluorophosphate

Z benzyloxycarbonyl

What is meant by “Acc” is an amino acid selected from the group of:

1-amino-1-cyclopropanecarboxylic acid (“A3c”);

1-amino-1-cyclobutanecarboxylic acid (“A4c”);

1-amino-1-cyclopentanecarboxylic acid (“A5c);

1-amino-1-cyclohexanecarboxylic acid (“A6c”);

1-amino-1-cycloheptanecarboxylic acid (“A7c”);

1-amino-1-cyclooctanecarboxylic acid (“A8c”); and

1-amino-1-cyclononanecarboxylic acid (“A9c”).

With the exception of the N-terminal amino acid, all abbreviations (e.g., Ala) of amino acids in this disclosure stand for the structure of —NH—C(R)(R′)—CO—, wherein R and R′ each is, independently, hydrogen or the side chain of an amino acid (e.g., R═CH₃ and R′═H for Ala). For the N-terminal amino acid, the abbreviation stands for the structure of:

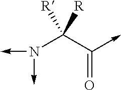

The designation “NH₂” in e.g., Ac-c(Cys-Lys-Trp-Ile-Gln-DCys)-NH₂ (SEQ ID NO:48), indicates that the C-terminus of the peptide is amidated. Ac-c(Cys-Lys-Trp-Ile-Gln-DCys) (SEQ ID NO:112), or alternatively Ac-c(Cys-Lys-Trp-Ile-Gln-DCys)-OH (SEQ ID NO:112), indicates that the C-terminus is the free acid.

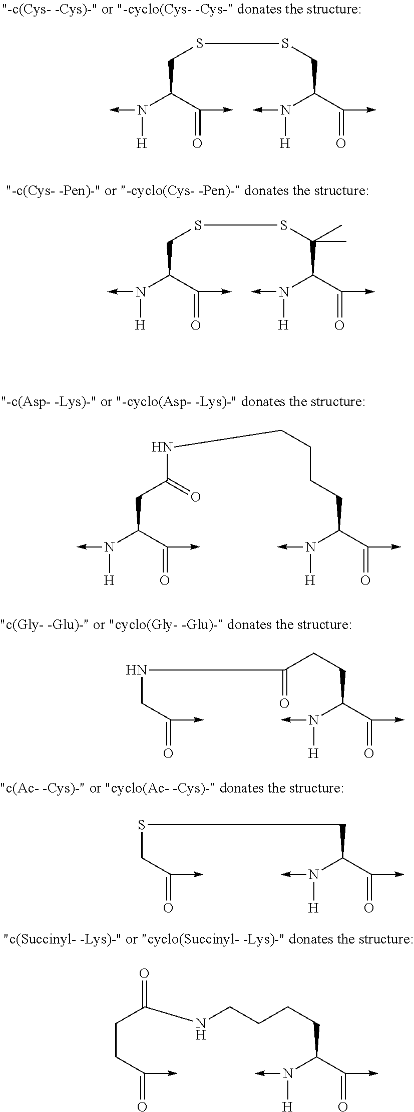

“Acyl” refers to R″—C(O)—, where R″ is H, alkyl, substituted alkyl, heteroalkyl, substituted heteroalkyl, alkenyl, substituted alkenyl, aryl, alkylaryl, or substituted alkylaryl.

“Alkyl” refers to a hydrocarbon group containing one or more carbon atoms, where multiple carbon atoms if present are joined by single bonds. The alkyl hydrocarbon group may be straight-chain or contain one or more branches or cyclic groups.

“Substituted alkyl” refers to an alkyl wherein one or more hydrogen atoms of the hydrocarbon group are replaced with one or more substituents selected from the group consisting of halogen (i.e., fluorine, chlorine, bromine, and iodine), —OH, —CN, —SH, —NH₂, —NHCH₃, —NO₂, —C₁₋₂₀ alkyl substituted with halogens, —CF₃, —OCH₃, —OCF₃, and —(CH₂)₀₋₂₀—COOH. In different embodiments, 1, 2, 3 or 4 substituents are present. The presence of —(CH₂)₀₋₂₀—COOH results in the production of an alkyl acid. Examples of alkyl acids containing, or consisting of, —(CH₂)₀₋₂₀—COOH include 2-norbornane acetic acid, tert-butyric acid and 3-cyclopentyl propionic acid.

“Heteroalkyl” refers to an alkyl wherein one or more of the carbon atoms in the hydrocarbon group are replaced with one or more of the following groups: amino, amido, —O—, —S—, or carbonyl. In different embodiments, 1 or 2 heteroatoms are present.

“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, —C₁₋₂₀ alkyl substituted with halogens, —CF₃, —OCH₃, —OCF₃, and —(CH₂)₀₋₂₀—COOH. In different embodiments, 1, 2, 3 or 4 substituents are present.

“Alkenyl” refers to a hydrocarbon group made up of two or more carbons where one or more carbon-carbon double bonds are present. The alkenyl hydrocarbon group may be straight-chain or contain one or more branches or cyclic groups.

“Substituted alkenyl” refers to an alkenyl wherein one or more hydrogens are replaced with one or more substituents selected from the group consisting of halogen (i.e., fluorine, chlorine, bromine, and iodine), —OH, —CN, —SH, —NH₂, —NHCH₃, —NO₂, —C₁₋₂₀ alkyl substituted with halogens, —CF₃, —OCH₃, —OCF₃, and —(CH₂)₀₋₂₀—COOH. In different embodiments, 1, 2, 3 or 4 substituents are present.

“Aryl” refers to an optionally substituted aromatic group with at least one ring having a conjugated pi-electron system, containing up to three conjugated or fused ring systems. Aryl includes carbocyclic aryl, heterocyclic aryl and biaryl groups. Preferably, the aryl is a 5 or 6 membered ring. Preferred atoms for a heterocyclic aryl are one or more sulfur, oxygen, and/or nitrogen. Examples of aryl include phenyl, 1-naphthyl, 2-naphthyl, indole, quinoline, 2-imidazole, and 9-anthracene. Aryl substituents are selected from the group consisting of —C₁₋₂₀ alkyl, —C₁₋₂₀ alkoxy, halogen (i.e., fluorine, chlorine, bromine, and iodine), —OH, —CN, —SH, —NH₂, —NO₂, —C₁₋₂₀ alkyl substituted with halogens, —CF₃, —OCF₃ and —(CH₂)₀₋₂₀-COOH. In different embodiments, the aryl contains 0, 1,2, 3, or 4 substituents.

“Alkylaryl” refers to an “alkyl” joined to an “aryl.”

“Chemokines” refers to a family of pro-inflammatory signaling molecules which act on macrophages, B cells, T cells, neutrophils, eosinophils, basophils, mast cells, smooth muscle cells, e.g., vascular smooth muscle cells, and the like (e.g., by affecting their migration, proliferation, or degranulation, or the immunomodulation of T cell development to Th1 and Th2 subtypes). Preferred chemokines are primate in origin, e.g., human, although the invention includes other mammalian chemokines, such as those of bovine, ovine, equine, canine, feline or rodent origin, as well as virally encoded chemokines.

Preferably, a peptide, variant, analog or derivative of the invention, has increased affinity for at least one chemokine receptor, e.g., about 1 μM to about 1 nM, more preferably about 1 nM to about pM, and also preferably has decreased Duffy binding, relative to a corresponding peptide having the native (“wild-type”) sequence or relative to the corresponding native chemokine. However, certain populations have individuals who are Duffy⁻, e.g., a certain percentage of African Americans are Duffy⁻. Thus, agents useful to treat these populations may have Duffy binding affinity that is equal to or greater than that of the corresponding native chemokine.

As used herein, “a chemokine-induced activity” includes, but is not limited to, an activity that is elicited through the binding of a chemokine, a therapeutic agent of the invention or other moiety, e.g., viral protein, to a chemokine receptor, or the binding of a therapeutic agent or other moiety in close physical proximity to the receptor so that the activity is altered. Chemokine receptors include, but are not limited to, CCR1, CCR2a, CCR2b, CCR3, CCR4, CCR5, CCR6, CCR7, CCR8, IL8R1, IL8R2, CC-CKRI, CC-CKR2, CC-CKR3, CXCR1, CXCR2, CXCR3, CX₃CR1 and CXCR4. Chemokine receptors play a role in cell migration, cell activation, viral or parasite entry, release of pro-inflammatory compounds, and the like.

As used herein, “indications associated with chemokine-induced activity” includes, but is not limited to, atherosclerosis and other forms of local or systemic vasculitis, diseases such as myocardial infarction, stroke and acute ischemia which are secondary to atherosclerosis; hypertension; reperfusion injury (Kumar et al., Circulation, 95:693, 1997); aortic aneurysms; vein graft hyperplasia; angiogenesis; hypercholesterolemia; congestive heart failure; Kawasaki's disease; stenosis or restenosis, particularly in patients undergoing angioplasty; pathologically low bone mineral density, such as osteoporosis (Posner et al., Bone, 21:321, 1997); ulcerative colitis; chronic obstructive pulmonary disease; infection with human immunodeficiency virus (HIV), other lentiviruses or retroviruses with similar mechanisms of cell entry via chemokine receptor(s), or infection with other viruses, e.g., cytomegalovirus (Sozzani et al., J. Leukoc. Biol., 62:30, 1997), or viral infection resulting in viral meningitis; organ transplantation, such as acute transplant rejection, allograft rejection and graft versus host disease; transplant vasculopathy; malaria and other consequences of infection by parasites related to plasmodium; asthma; allergic diseases, such as atopy (IgE-mediated components), allergic rhinitis, atopic dermatitis, anaphylaxis, allergic bronchopulmonary aspergillosis (IgE-mediated), and hypersensitivity pneumonitis (high IgG and reactive T cells) (pigeon breeders disease, farmer's lung disease, humidifier lung disease, malt workers' lung disease); allergies, including flea allergy dermatitis in mammals such as domestic animals, e.g., dogs and cats, contact allergens including mosquito bites or other insect sting allergies, poison ivy, poison oak, poison sumac, or other skin allergens; urticaria; eczema; pulmonary fibrosis such as an idiopathic pulmonary fibrosis; cystic fibrosis; hemolytic uremic syndrome (Van Setten et al., Pediatr. Res., 43:759, 1998); autoimmune disorders, including, but not limited to, type I diabetes, Crohn's disease, multiple sclerosis, arthritis, rheumatoid arthritis (Ogata et al., J. Pathol., 182:106, 1997; Gong et al., J. Exp. Med., 186:131, 1997), systemic lupus erythematosus, autoimmune (Hasimoto's) thyroiditis, autoimmune liver diseases such as hepatitis and primary biliary cirrhosis, hyperthyroidism (Graves' disease; thyrotoxicosis), insulin-resistant diabetes, autoimmune adrenal insufficiency (Addison's disease), autoimmune oophoritis, autoimmune orchitis, autoimmune hemolytic anemia, paroxysmal cold hemoglobinuria, Behcet's disease, autoimmune thrombocytopenia, autoimmune neutropenia, pernicious anemia, pure red cell anemia, autoimmune coagulopathies, myasthenia gravis, autoimmune polyneuritis, experimental allergic encephalomyelitis, pemphigus and other bullous diseases, rheumatic carditis, Goodpasture's syndrome, postcardiotomy syndrome, Sjogren's syndrome, polymyositis, dermatomyositis, and scleroderma; eye diseases such as uveitis or blinding Herpes stromal keratitis; liver disease; erhlichiosis or Lyme disease including Lyme arthritis; aberrant hematopoiesis; nephritis due to, for example, autosomal dominant polycystic kidney disease, diabetic nephropathy, IgA nephropathy, interstitial fibrosis, or lupus; as well as other disease states resulting from inappropriate inflammation, either local or systemic, for example, irritable or inflammatory bowel syndrome (Mazzucchelli et al., J. Pathol., 178:201, 1996), psoriasis (Gillitzer et al., Arch. Dermatol. Res., 284:26, 1992; Yu et al., Lab Investig., 71:226, 1994), delayed type hypersensitivity, Alzheimer's disease, chronic pulmonary inflammation, e.g., pulmonary alveolitis and pulmonary granuloma, gingival inflammation or other periodontal disease, and osseous inflammation associated with lesions of endodontic origin (Volejnikova et al., Am. J. Pathol., 150:1711, 1997), hypersensitivity lung diseases such as hypersensitivity pneumonitis (Sugiyama et al., Eur. Respir. J., 8:1084, 1995), an inflammation related to histamine release from basophils (Dvorak et al., J. Allergy Clin. Immunol., 98:355, 1996), such as hay fever, histamine release from mast cells (Galli et al., Ciba Foundation Symposium, 147:53, 1989), or mast cell tumors, types of type 1 hypersensitivity reactions (anaphylaxis, skin allergy, hives, allergic rhinitis, and allergic gastroenteritis); glomerulonephritis (Gesualdo et al., Kidney International, 51:155, 1997); inflammation associated with peritoneal dialysis (Sach et al., Nephrol. Dial. Transplant, 12:315, 1997); and pancreatitis.

Other indications falling within the scope of the invention include, but are not limited to, neoplasia, e.g., histocytoma, glioma, sarcoma, osteosarcoma, osteoma (Zheng et al., J. Cell Biochem., 70:121, 1998), melanoma, Kaposi's sarcoma, small cell lung cancer, and ovarian carcinoma as well as myelosuppression and mucositis associated with chemotherapy; brain or spinal cord trauma, such as after disc surgery (Ghirnikar et al., J. Neurosci. Res., 46:727, 1996; Berman et al., J. Immunol., 156:3017, 1996); gout; lung disease, e.g., due to respiratory syncicial virus infection of humans, cattle, pigs and the like, or lung injury (Lukacs et al., Adv. Immunol., 62:257, 1996); strokes; Loeffler's syndrome; chronic eosinophilic pneumonia; pulmonary fibrosis; would healing; bacterial infection, e.g., bacterial peritonitis or meningitis; granulomatous diseases such as Mycobacteriosis, Pneumocystosis, Histoplasmosis, Blastomycosis, Coccidiomycosis, Cryptococcosis, Aspergillosis, granulomatous enteritis, Candidiasis, foreign body granulomas and peritonitis, pulmonary granulomatosis, Wegener's granulomatosis (Del Papa et al., Arthritis Rheum., 39:758, 1996), leprosy, syphilis, cat-scratch disease, schistosomiasis (Jacobs et al., Am. J. Pathol., 150:2033, 1997), silicosis, sarcoidosis (Iida et al., Thorax, 52:431, 1997; Car et al., Am. J. Respir. Crit. CareMed., 149:655, 1994) and berylliosis; lethal endotoxemia (Zisman et al., J. Clin. Invest., 99:2832, 1997); and indications associated with a weak inflammatory response, e.g., which occur in parasitic infection, e.g., Leishmaniasis (Moll, Biol. Abs., 104:21765, 1997), trypanosome, Mycobacterium leprae or Mycobacterium tuberculosis infection, helminth infections, such as nematodes (round worms) (Trichuriasis, Enterobiasis, Ascariasis, Hookworm, Strongyloidiasis, Trichinosis, filariasis); trematodes (fluxes) (Schistosomiasis, Clonorchiasis), cestode (tape worms) (Echinococcosis, Taeniasis saginata, Cysticercosis); visceral works, visceral larva migrans (e.g., Toxocara), eosinophilic gastroenteritis (e.g., Anisaki spp., Phocanema ssp.), cutaneous larva migrans (Ancylostoma braziliense, Ancylostoma caninum), or fungal infection.

In addition, to prevent or treat indications associated with a weak inflammatory response, the agents of the invention may be employed as vaccine adjuvants.

The peptides of the invention may also be useful as contraceptives or to induce abortion, in acute respiratory distress syndrome, and diseases where steroids are routinely used (e.g., relapsing Beheers colitis and asthma).

Also included within the scope of the invention are indications associated with tumor necrosis factor α (TNFα), e.g., rheumatoid arthritis or endotoxemia, or indications associated with elevated levels of TNFα. These indications include, but are not limited to, endotoxic shock; Crohn's disease; fever, and flu-like symptoms; acute interstitial pneumonitis; septic and nonseptic shock; acute respiratory distress syndrome; thromboembolic conditions; bone resorption; arthritis; acute graft versus host disease; cerebral malaria; cachexia of tuberculosis or cancer; lung injury; and idiopathic fibrosis.

Identification of Therapeutic Agents Falling within the Scope of the Invention

Agents useful in the practice of the invention include agents that inhibit or reduce (e.g., chemokine receptor antagonists), or increase, augment or enhance (e.g., chemokine receptor agonists), chemokine-induced activity, e.g., monocyte or macrophage recruitment. These agents can be identified in vitro or in vivo assays, such as the assays described hereinbelow. It is recognized that not all agents falling within the scope of the invention can inhibit or enhance chemokine-induced activity in vitro or in vivo. The therapeutic agents of the invention may be direct receptor binding agonists and/or antagonists, or may act by a different mechanism, e.g., duplex formation of antisense nucleic acid with chemokine mRNA, or by more than one mechanism, so as to result in the alteration of chemokine-induced activity.

In vitro Chemotaxis

To determine whether an agent inhibits a chemokine-induced activity, such as macrophage recruitment, varying amounts of the agent are mixed with cells in the presence of a known chemoattractant. For example, a range of known concentrations of an agent, e.g., a chemokine peptide, is incubated with a defined number (e.g., 10⁴-10⁶) of human THP-1 monocyte cells in individual wells of the top compartment of a trans-well plate. Chemokine, at a concentration known to cause significant migration of THP-1 cells in the trans-well migration assay, is placed in the lower compartment of a trans-well plate. Cells are then incubated at 37° C. for a period sufficient to allow migration, e.g., 4 hours. After incubation, the cells are gently removed from the top of the filter with a pipette, 20 μl of 20 mM EDTA in simple PBS is added into each top well, and incubated for 20 minutes at 4° C. The filter is carefully flushed with media using a gentle flow, and removed. A standard curve consisting of a two-fold dilution series of THP-1 cells (in 29 μl) is placed to accurately quantify the number of cells that have migrated. Migrated cells are stained with 3 μl of MTT stock dye solution which is added directly into each well (5 mg/ml in RPMI-1640 without phenol red) (Sigma Chemical Co., St. Louis, Mo., USA) and incubated at 37° C. for 4 hours. The media is carefully aspirated from each well, and the converted dye is solubilized by 20 μl of DMSO. Absorbance of converted dye is measured at a wavelength of 595 nm using an ELISA plate reader. The number of migrated cells in each well is then determined by interpolation of the standard curve (see also Imai et al., J. Biol. Chem., 272:15036, 1997).

Any method suitable for counting cells can be used, for example, counting with a hemocytometer, incubation of the cells with MTT (see above), or FACS analysis. A negative control assay is also performed, using TGFβ or another non-chemokine chemoattractant (e.g., IL1β or TNFα). To assess whether the agent is cytotoxic, the same concentrations of agent are incubated with THP-1 cells. Agents which 1) are not cytotoxic at levels which inhibit migration, 2) are ineffective at inhibiting the negative control-induced migration, and 3) reduce or inhibit chemokine-induced THP-1 migration, are agents which fall within the scope of the invention.

Agents may also be screened in a chemotactic assay which employs human neutrophils, eosinophils, mast cells, basophils, platelets, lymphocytes or monocytes. For monocytes, 9 ml of fresh blood are transferred to a tube containing 1 ml of 3.8% sodium citrate, and left at room temperature for 15 minutes. 5 ml of this anti-coagulated blood are carefully layered over 3.5 ml Polymorphprep® (Nycomed Pharma, Oslo, Norway), and centrifuged at 500 g for 35 minutes per the manufacturer's instructions. The top band at the sample/medium interface contains monocytes. The monocytes are carefully removed with a glass pipette, and reconstituted to the original volume (5 ml). The cells are washed with PBS plus 10% fetal calf serum, and centrifuged at 400 g for 10 minutes. The washing step is repeated three times before the cells are counted. Cells are resuspended at 1×10⁷ cells/ml in RPMI-1640+10% FCS. The monocytes are cultured for two days at 37° C. in a humidified atmosphere of 5% CO₂.

On day 2, the cells are counted, spun down, and reconstituted to 1×10⁷ cells/ml in Gey's balanced salt solution+1 mg/ml BSA. Chemotaxis is induced in a 48 or 96-well disposable chemotaxis chamber fitted with a 5-8 μm polycarbonate filter for monocytes, neutrophils or eosinophils, or a 3 μm filter for lymphocytes (Uguccioni et al., Eur. J. Immunol., 25:64, 1995; Loetscher et al., J. Exp. Med., 184:569, 1996; Weber et al., J. Immunol., 4166, 1995) (PVP free) (ChemoTX, Neuroprobe Inc., Cabin John, MD, USA). 29 μl of chemoattractant or control are added to the lower compartment of each well. The framed filter is aligned with the holes in the corner of the filter frame and place over the wells. 2½×10⁵ monocytes in 25 μl of Gey's balanced salt solution+1 mg/ml BSA are added to the upper compartment. The agent is dissolved in Milli Q water and then serially diluted in the Gey's balanced salt solution. In most cases, the serially diluted agent is added to the upper compartment of the chemotaxis chamber. The chamber is incubated at 37° C. in a humidified atmosphere of 5% CO₂ for 1.5 hours.

Enzyme Release

The release of N-acetyl-β-D-glucosaminidase from monocytes may be employed to determine whether a therapeutic agent inhibits a cytokine-associated activity. Samples of 1.2×10⁶ monocytes in 0.3 ml of prewarmed medium (136 mM NaCl, 4.8 mM KCl, 1.2 mM KH₂PO₄, 1 mM CaCl₂, 20 mM HEPES, pH 7.4, 5 mM D-glucose, and 1 mg/ml fatty acid-free BSA) are pretreated for 2 minutes with cytochalasin B (2.7 mg/ml) and then stimulated with a chemokine in the presence or absence of the therapeutic agent. The reaction is stopped after 3 minutes by cooling on ice and centrifugation, and the enzyme activity is determined in the supernatant (Uguccioni et al., Eur. J. Immunol., 25:64, 1995).

The release of elastase from neutrophils may also be employed to determine whether a therapeutic agent inhibits a cytokine-associated activity (Pereri et al., J. Exp. Med., 1547, 1988; Clark-Lewis et al., J. Biol. Chem., 269:16075, 1994).

Cytosolic Free Ca²⁺ Concentration [Ca²⁺]ᵢ Changes

Monocytes, eosinophils, neutrophils and lymphocytes loaded with Fura-2 (0.1 nmol/10⁵ cells) are stimulated with a chemokine in the presence or absence of the therapeutic agent, and [Ca²⁺]ᵢ-related fluorescence changes are recorded (Von Tschanner et al., Nature, 324:369, 1986). For example, to determine cytosolic Ca²⁺ concentrations in monocytes, monocytes are incubated with 0.5 μM Fura-2/AM for 30 minutes at 37° C. in HEPES-buffered saline (145 mM NaCl, 5 mM KCl, 1 mM MgCl₂, 10 mM HEPES, and 10 mM glucose), pH 7.4, at 37° C., supplemented with 1% albumin (w/v) and 1 mM CaCl₂. After loading with Fura-2, the cells are centrifuged for 5 minutes at 300 g and then resuspended in buffer containing no added albumin, to a cell density of 1.5×10⁶ cells/ml, and kept at room temperature until use. This protocol results in a cytosolic Fura-2 concentration of about 100 μM. Serial dilutions of chemokines in PBS plus 0.1% albumin (w/v) (sterile filtered) are added to aliquots (0.7 ml) of cell suspension. The Fura-2 fluorescence of the monocyte suspension is measured at 37° C. in a single excitation, single emission (500 nm) wavelength Perkin-Elmer LS5 fluorometer (Perkin-Elmer, http://www.perkinelmer.com). [Ca²⁺]ᵢ is calculated from changes in fluorescence measured at a single excitation wavelength of 340 nm.

[Ca²⁺]ᵢ measurements in cells that are stably transformed with a molecularly cloned chemokine receptor which is not expressed in the corresponding non-transformed cells are performed essentially as described above. After loading with Fura-2/AM, cells (1×10⁶/ml) are kept in ice-cold medium (118 mM NaCl, 4.6 mM KCl, 25 mM NaHCO₃, 1 mM KH₂PO₄, 11 mM glucose, 50 mM HEPES, 1 mM MgCl₂, 1 mM CaCl₂, 0.1% gelatin (pH 7.4). Aliquots (2 ml) of cell suspension are prewarmed at 37° C. for 5 minutes in 3-ml plastic cuvettes, and fluorescence is measured in a fluorometer (Johnson Foundation Biomedical Group) with magnetic stirring and temperature controlled at 37° C. Excitation is set at 340 nm, and emission is set at 510 nm. [Ca²⁺]ᵢ is calculated as described above.

For studies in monocytes on cross-desensitization of calcium responses, chemokines are added sequentially with a 2-minute interval, and [Ca²⁺]ᵢ transients are recorded. The concentrations used in these types of studies vary for each chemokine and re set at levels known to induce the maximal response for [Ca²⁺]ᵢ mobilization (see Forssmann et al., FEBS Lett., 408:211, 1997; Sozzani et al., J. Leukoc. Biol., 57:788, 1995; Berkhout et al., J. Biol. Chem., 272:16404, 1997).

Chemokine Binding and Binding Displacement

In general, specific binding is calculated as the amount of labeled agent bound in the absence of cold competitor minus the amount of labeled agent bound in the presence of cold competitor. The amount of specific binding in the presence of varied amounts of cold competitor can be used to determine the association constant for the agent, as well as the number of binding sites on the cell for the agent, using, for example, Scatchard Analysis. The agent may be labeled by radiolabeling (e.g., iodination) or with a suitable biochemical tag (e.g., biotin) or by addition of a photoactivatable crosslinking group. Agents with an association constant lower than 100 μM (i.e., which bind more strongly than an agent with an association constant of 100 μM) and which have at least about 2,500, preferably at least 10,000, and more preferably greater than 25,000, binding sites per cell for at least one cell type which expresses a chemokine receptor, fall under the scope of this invention. THP-1 cells have at least about 5,000 MCP-1 receptors/cell.

For example, monocytes are suspended in RPMI 1640 medium without bicarbonate containing 0.2% bovine serum albumin and 0.1% azide. Radiolabeled chemokine peptide is incubated with 1-2×10⁶ cells, e.g., THP-1 cells, in the presence or absence of increasing concentrations of unlabeled chemokine for 15 minutes at 37° C. in a 96-well plate in a final volume of 0.2 ml (e.g., PBS+0.5% FCS). After the incubation, 0.5 ml of ice-cold wash buffer (20 mM Tris, 0.5 M NaCl, pH 7.4) is added, and cells are collected onto a polyethyleneimine-treated Whatman GF/C filter using a Brandall cell harvester. Filters are washed with 4 ml of cold wash buffer, and the radioactivity bound to the filters is countered in a γ-counter.

For competition studies, the IC₅₀ is calculated with a curve fitting program (GraFit, Erithacus Software, London, U.K.), using a four-parameter logistic, cpm_{bound}=cpmₘₐₓ/(1+([L]/IC₅₀)^{s})=cpmₙₛ, where cpmₘₐₓ represents the binding without competitor, [L] is the competitor, cpmₙₛ is the non-specific binding, and s is the slope factor. The cpm_{bound} is corrected for “no cell” controls. To obtain the IQ and capacity of specific binding, data from homologous displacement experiments are fitted into a single-site ligand binding equation using the GraFit best fit program.

Chemokine binding to cells stably transformed with a molecularly cloned chemokine receptor is performed essentially as described above except that radiolabeled agent is diluted with unlabeled chemokine. Cells are incubated with radiolabeled agent plus or minus unlabeled chemokines for 30 minutes at 37° C. (see also Imai et al., supra; Sozzani et al., supra; Berkhout et al., supra; WO 97/22698).

Binding to the Duffy Antigen Receptor for Chemokines (DARC)

The affinity of the therapeutic agent to DARC may be determined to any method known in the art. Agents which bind to DARC with a lower association constant (i.e., stronger binding) than they bind to chemokine receptors (i.e., a DARC selectivity ration of <1), and which bind to DARC with an association constant lower than 100 μM, preferably lower than 10 μM and more preferably lower than 1 μM, are useful in particular embodiments of the methods of the invention. In contrast, agents which do not bind DARC, or do not bind to DARC with an affinity that is greater than their affinity for chemokine receptors (i.e., a selectivity ration of >1), are useful in the practice of other embodiments of the methods of the invention.

Inhibition of the Co-Mitogenic Activity of Chemokines

Many chemokines are co-mitogenic with low concentrations of FCS. Assays well known in the art for determination of DNA synthesis induced by any known chemokine plus a low concentration (<5%) of FCS on suitable cells (e.g., smooth muscle cells) in the presence or absence of the agent may be employed to screen agents for such inhibitory activity. See Porreca et al., J. Vasc. Res., 34:58, 1997, the disclosure of which in incorporated by reference herein.

Anti-Lentiviral Activity

To prepare cell lines that are susceptible to lentiviral infection as a result of the expression of a particular chemokine receptor, a molecularly cloned chemokine receptor is introduced into a cell line that does not otherwise express the chemokine receptor, e.g., HeLa-MAGI (Kimpton and Emerman, J. Viral., 66(5):3026, 1992) or U373-MAGI (Harrington and Geballe, J. Viral., 67:5939, 1993) cells, by infection with a retroviral vector. Expression of the chemokine receptor on the cell surface is demonstrated by immunostaining live cells using antibody. Expression of the RNA encoding the receptor is demonstrated by RT-PCR analysis. HeLa-MAGI and U373-MAGI express β-galactosidase after lentiviral infection. Incubation of infected cells with X-gal results in the deposit of a blue stain in these cells.

Infection of the chemokine receptor-stably transformed cell lines with HIV in the presence or absence of agent is performed in 12-well plates with 10-fold serial dilutions of 300 μl of virus in the presence of 30 μg/ml DEAE-Dextran as described (Kimpton and Emerman, supra). Viral stocks are normalized by ELISA or p24^{gag} (Coulter Immunology, Miami, Fla., USA) or p27^{gag} (Coulter Immunology, Miami, Fla., USA) for HIV-1 and HIV-2/SIV, respectively, using standards provided by the manufacturer.

Two days after infection, cells are fixed and stained for (3-galactosidase activity with X-gal. The cells are stained for 50-120 minutes at 37° C. The infectious titer is the number of blue cells per well multiplied by the dilution of virus and normalized to 1 ml.

For other methods useful to determine whether an agent inhibits lentiviral infection and/or replication, see also Cocchi et al., Science, 270:1811, 1995, and W097/22698.

Agonists

To determine whether an agent of the invention is a chemokine receptor agonist, varying amounts of a labeled form of the agent, e.g., biotinylated, are mixed with cells that express the receptor. The affinity of the labeled agent for the cells is then determined. Agents that bind to receptors with a reasonable affinity and interact with the receptor by inducing signaling, are within the scope of the invention. While not encompassed by the term “agonist” or “antagonist”, agents that bind to or near the receptor but elicit no response are also within the scope of the invention, and are termed “neutral” agents.

Agents with agonist activity may also be identified using the transwell migration assay, where the cells are placed in the upper compartment in the absence of agent, and the agent is placed at varying concentrations in the lower compartment in place of the chemokine. If the agent(s) have agonist activity, more cells are found in the lower compartment at the end of the assay in wells containing the agent(s) than in wells containing inactive control, i.e., agent or medium alone. Preferably, agents having agonist activity also stimulate migration of primary human cells, e.g., monocyte, in a transwell migration assay.

Moreover, weak agonists or neutral agonists (agents which bind to the receptor but do not inhibit binding of native chemokine and its subsequent signaling, nor do they induce signaling themselves) can be identified by screening the agents for ability to displace the binding of HIV gp120, specifically the V3 loop of gp120, to the surface of THP-1 cells or Jurkat cells. Cells are incubated with labeled (for example, radioiodinated) recombinant gp120 protein in an amount effective to bind to the virus receptor, in the presence or absence of various concentrations of the agent(s). Agents which reduce or abolish gp120 binding are agonists or neutral agents within the scope of the invention.

In vivo Studies

A rapid method to determine whether an agent of the invention inhibits or augments an inflammatory response is to inject a selected chemokine into the skin of an animal in the presence or absence of an agent of the invention. At some later point in time, animals are sacrificed and the number of inflammatory cells in animals exposed to chemokine and the agent is compared to the number of inflammatory cells in animals to chemokine alone, e.g., by quantitative immunofluorescence, relative to control animals.

Peptide Synthesis

The present isolated, purified chemokine peptides, peptide variants or derivatives thereof, can be synthesized in vitro, e.g., by the solid phase peptide synthetic method. The solid phase peptide synthetic method is an established and widely used method, which is described in the following references: Stewart et al., Solid Phase Peptide Synthesis, W. H. Freedman Co., San Francisco, 1969; Merrifield, J. Am. Chem. Soc., 85:2149, 1963; Meienhofer in “Hormonal Proteins and Peptides,” ed.; C. H. Li, Academic Press, 2:48-267, 1973; Bavaay and Merrifield, “The Peptides,” eds. E. Gross and F. Meienhofer, 2:3-285, 1980; and Clark-Lewis et al., Meth. Enzymol., 287:233, 1997. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given chemokine peptide can be readily prepared. For example, amides of the chemokine peptide or chemokine peptide variants of the present invention may also be prepared by techniques well known in the art for converting a carboxylic acid group or precursor, to an amide.

Salts of carboxyl groups of a peptide or peptide variant of the invention may be prepared in the usual manner by contacting the peptide with one or more equivalents of a desired base such as, for example, a metallic hydroxide base, e.g., sodium hydroxide, a metal carbonate or bicarbonate base such, for example, sodium carbonate or sodium bicarbonate; or an amide base such as, for example, triethylamine, triethanolamine, and the like.

N-acyl derivatives of an amino group of the chemokine peptide or peptide variants may be prepared by utilizing an N-acyl protected amino acid for the final condensation, or by acylating a protected or unprotected peptide. O-acyl derivatives may be prepared, for example, by acylation of a free hydroxyl peptide or peptide resin. Either acylation may be carried out using standard acylating reagents such as acyl halide, anhydrides, acyl imidazoles, and the like. Both N- and O-acylation may be carried out together, if desired.

In addition, the amino acid sequence of a chemokine peptide can be modified so as to result in a chemokine peptide variant. The modification includes the substitution of at least one amino acid residue in the peptide for another amino acid residue, including substitutions which utilize the D rather than L form, as well as other well known amino acid analogs, e.g., unnatural amino acids such as a-disubstituted amino acids, N-alkyl amino acids, lactic acid, and the like.

The substituents R² and R³ of the above generic formula may be attached to the free amine of the N-terminal amino acid by standard methods known in the art. For example, alkyl groups, e.g., (C₁-C₃₀)alkyl, may be attached using reductive alkylation. Hydroxyalkyl groups, e.g., (C₁-C₃₀)hydroxyalkyl, may also be attached using reductive alkylation wherein the free hydroxy group is protected with a t-butyl ester. Acyl groups, e.g., COE¹, may be attached by coupling the free acid, e.g., E¹COOH, to the free amine of the N-terminal amino acid by mixing the completed resin with 3 molar equivalents of both the free acid and diisopropylcarbodiimide in methylene chloride for one hour. If the free acid contains a free hydroxy group, e.g., p-hydroxyphenylpropionic acid, then the coupling should be performed with an additional 3 molar equivalents of HOBt.

When R¹ is —NH₂, the synthesis of the peptide starts with an Fmoc-amino acid which is coupled to the Rink Amide MBHA resin. If R¹ is —OH, the synthesis of the peptide starts with a Fmoc-amino acid which is coupled to Wang resin.

### Example 1

### c(Cys-Lys-Trp-Ile-Gln-Cys)-NH₂ (SEQ ID NO:38)

The title peptide was synthesized on an Advanced ChemTech model 396 multiple peptide synthesizer (the “ACT 396 multiple peptide synthesizer”) (Advanced ChemTech, Louisville, Ky., USA) using Fluorenylmethyloxycarbonyl (Fmoc) chemistry. A Rink Amide 4-methylbenzylhydrylamine (MBHA) resin (Novabiochem, San Diego, Calif., USA) with substitution of 0.58 mmol/g was used. The Fmoc amino acids (Novabiochem, San Diego, Calif., USA, and Chem-Impex, Wood Dale, Ill., USA) used were Fmoc-Cys(Trt)-OH, Fmoc-Lys(Boc)-OH, Fmoc-Trp(Boc)-OH, Fmoc-Ile-OH, and Fmoc-Gln(Trt)-OH. The synthesis was carried out on a 0.090 mmol scale. The Fmoc groups were removed by treatment with 25% piperidine in N,N-dimethylformamide (DMF) for 5 min. Then a second deprotection was done for 25 min. In each coupling step, the Fmoc amino acid (4 eq, 0.36 mmol), N,N-diisopropylcarbodiimide (DIC) (4 eq, 0.36 mmol), and 1-hydroxy-benzotriazole (HOBt) (4 eq, 0.36 mmol) in DMF (1.5 mL) were used. Double coupling was performed for each residue.

The ACT 396 multiple peptide synthesizer was programmed to perform the following reaction cycle: 1) washing with DMF, 2) removing Fmoc protecting group with 25% piperidine in DMF for 5 and 25 minutes, 3) washing with DMF, 4) coupling with Fmoc amino acid in the presence of DIC and HOBt for 1 hour, 5) washing with DMF, and 6) repeat step 4. The resin was coupled successively according to the sequence of the title peptide. After the peptide chain was assembled and the last Fmoc-protecting group was removed, the resin was washed completely by using DMF and dichloromethane (DCM).

To cleave the peptide off the resin, the peptide-resin was treated with a TFA solution containing 8% triisopropylsilane (TIS) for 2 hours at room temperature. The resin was filtered off and the filtrate was poured into 25 mL of ether. The precipitate was collected by centrifugation. This crude product was dissolved in 0.2N NH₄OAc aqueous solution (180 mL) and the pH of the solution was adjusted to ˜8.5 by adding concentrated NH₄OH. The solution was opened to the air for 72 hours at room temperature. The solution was acidified with HOAc after the oxidation reaction was complete.

The resulting crude product was purified on a reverse-phase preparative HPLC system with a column (4×43 cm) of C₁₈ DYNAMAX-100 A⁰ (Varian, Walnut Creek, Calif., USA). The column was eluted over approximately 1 hour using a linear gradient of 85% A: 15% B to 30% A: 70% B, where A was 0.1% TFA in water and B was 0.1% TFA in acetonitrile. The fractions were checked by analytical HPLC and those containing pure product were pooled and lyophilized to dryness to give 31.5 mg (45% yield) of a white solid. Purity was 95.4% based on the analytical HPLC analysis. Electro-spray ionization mass spectrometry (ESI-MS) analysis gave the molecular weight at 776.3 (in agreement with the calculated molecular weight of 776.98).

### Example 2

### c(Cys-Trp-Ile-Gln-Cys)-NH₂ (SEQ ID NO:47)

The title peptide was synthesized by using a synthetic procedure analogous to the one described in Example 1. From 0.068 mmol Rink Amide MBHA resin, 13.1 mg of the desired product was obtained (yield=30%). Purity was 99% based on the analytical HPLC analysis. Electro-spray ionization mass spectrometry (ESI-MS) analysis gave the molecular weight at 648.2 (in agreement with the calculated molecular weight of 648.81).

### Example 3

### Ac-DGln-c(Cys-Lys-Trp-Ile-Gln-Cys)-NH₂ (SEQ ID NO:39)

The title peptide was synthesized by using a synthetic procedure analogous to the one described in Example 1. From 0.090 mmol Rink Amide MBHA resin, 31.5 mg of the desired product was obtained (yield=37%). Purity was 91.3% based on the analytical HPLC analysis. Electro-spray ionization mass spectrometry (ESI-MS) analysis gave the molecular weight at 946.5 (in agreement with the calculated molecular weight of 947.15).

### Example 4

### (Ac=Lys=Trp=Ile=Gln=hCyc)=NH₂ (SEQ ID NO:57)

The title peptide was synthesized using Fmoc-chemistry on an Advanced ChemTech Apex peptide synthesizer (Advanced ChemTech, Louisville, Ky., USA). 150 mg of 0.78 mmol/g (0.117 mmol) Rink Amide MBHA resin (Novabiochem, San Diego, Calif., USA) was placed in a reaction well and pre-swollen in 1.5 mL of dimethylformamide (DMF) prior to synthesis. Cycle 1: The resin was treated with two 1.5 mL portions of 25% piperidine in DMF for 5 and 25 minutes, respectively, followed by 4 washes of 1.5 mL DMF. Amino acids stocks were prepared in N-methylpyrrollidinone (NMP) as 0.45N solutions containing 0.45N HOBt. DIC was prepared as a 1.8N solution in NMP. To the resin 1.55 mL of the first amino acid (0.7 mmol), Fmoc-homocysteine(Trt)-OH (Bachem, Torrance, Calif., USA), was added along with 0.39 mL (0.7 mmol) of DIC. After one hour of constant mixing, the coupling reagents were drained from the resin and the coupling step repeated. The resin was washed with two 1.5 mL aliquots of DMF for 1 minute. The process of assembling the peptide (deblock/wash/acylate/wash) was repeated for cycles 2-5 identical to that as described for cycle 1. The following amino acids were used: for cycle 2, Fmoc-Gln(Trt)-OH (Genzyme, Cambridge, Mass., USA); for cycle 3, Fmoc-Ile-OH (Novabiochem, San Diego, Calif., USA); for cycle 4, Fmoc-Trp(Boc)-OH (Genzyme, Cambridge, Mass., USA); and for cycle 5, Fmoc-Lys(Boc)-OH (Novabiochem, San Diego, Calif., USA). The N-terminal Fmoc was removed with 25% piperidine in DMF, followed by four 1.5 mL DMF washes for 1 minute. To the resin 1.5 mL of 1N bromoacetic acid in NMP plus 0.5 mL of 1N DIC in NMP was added. The resin was mixed for 30 minutes, the well emptied, and the acetylation procedure repeated. The resin was washed with DMF, followed by 3 washes with dichloromethane (DCM).

To the resin, 5 mL of the following reagent was added: 2% TIS, 5% water, 5% (w/v) dithiothrieitol (DTT), 88% TFA, and allowed to mix for 3.5 hours. The filtrate was collected into 40 mL of cold anhydrous ethyl ether. The precipitate was pelleted for 10 minutes at 3500 RPM in a refrigerated centrifuge. The ether was decanted, and the peptide re-suspended in fresh ether. The ether workup was performed a total of 3 times. Following the last ether wash the peptide was allowed to air dry to remove residual ether. The peptide was dissolved in 10% acetonitrile and analyzed by mass spectrometry and reverse-phase HPLC. HPLC analysis employing a 30×4.6 cm C18 column (Vydac) using a gradient of 2-60% acetonitrile (0.1% TFA) over 30 minutes. This analysis identified a product with >75% purity. Mass analysis employing electrospray ionization identified a main product containing a mass of 812.5 Da, which corresponded to the desired Br-peptide product. The crude product (˜95 mg) was diluted to a concentration of 1 mg/mL in 10% DMSO, the pH was adjusted to 6.0 with saturated ammonium carbonate in water. After 16 hours the peptide was re-examined by mass spectrometry analysis and HPLC. Mass spectrometery analysis identified a single species with a mass of 730.3, suggesting the Br was successfully displaced. The peptide solution was concentrated to ˜10 mL using a Genevac concentrator, and then purified on a preparative HPLC equipped with a C18 column using a similar elution gradient. The fractions were checked by analytical HPLC and those containing pure product were pooled and lyophilized to dryness to give 25.8 mg (30% yield) of a white solid. Purity was 95% based on the analytical HPLC analysis. Electro-spray ionization mass spectrometry (ESI-MS) analysis gave the molecular weight at 730.3 (in agreement with the calculated molecular weight of 729.90).

### Example 5

### c(Ahx-Lys-Trp-Ile-Gln-Asp)-NH₂ (SEQ ID NO:41)

The title peptide was synthesized using Fmoc-chemistry on an Advanced ChemTech Apex peptide synthesizer (Advanced ChemTech, Louisville, Ky., USA). 115 mg of 0.78 mmol/g (0.09 mmol) Rink Amide MBHA resin (Novabiochem, San Diego, Calif., USA) was placed in a reaction well and pre-swollen in 1.5 mL of DMF prior to synthesis. Cycle 1: The resin was treated with two 1.5 mL portions of 25% piperidine in DMF for 5 and 25 minutes respectively, followed by 4 washes of 1.5 mL DMF, mixing for 1 minute, emptying for 1 minute. Amino acids stocks were prepared in NMP as 0.45N solutions containing 0.45N HOBt. DIC was prepared as a 0.75N solution in NMP. To the resin 1.1 mL of the first amino acid (0.49 mmol), Fmoc-Asp(O-2-PhiPr)-OH, (Novabiochem, San Diego, Calif., USA) was added along with 0.65 mL (0.49 mmol) of DIC. After one hour of constant mixing the coupling reagents were drained from the resin, and the coupling step repeated. The resin wash washed with two 1.5 mL aliquots of DMF for 1 minute. The process of assembling the peptide (deblock/wash/acylate/wash) was repeated for cycles 2-5 identical to that as described for cycle 1. The following amino acids were used: for cycle 2, Fmoc-Gln(Trt)-OH (Genzyme, Cambridge, Mass., USA); for cycle 3, Fmoc-Ile-OH (Novabiochem, San Diego, Calif., USA); for cycle 4, Fmoc-Trp(Boc)-OH (Genzyme, Cambridge, Mass., USA); for cycle 5, Fmoc-Lys(Boc)-OH (Novabiochem, San Diego, Calif., USA); and for cycle 6, Fmoc-Ahx-OH (Advanced ChemTech, Louisville, Ky., USA). The N-terminal Fmoc was removed with 25% piperidine in DMF, followed by four 1.5 mL DMF washes for 1 minute, and 3 washes with DCM.

The side-chain protecting group of Asp was deblocked using ten, 5 mL washes of 5% TIS, 1% TFA in DCM for 2 minutes. Following the last wash the resin was washed with DCM and DMF twice, respectively. The resin was treated with 25% piperdine twice for 2 minutes, followed by 4 DMF washes. An aliquot of resin (<1 mg) gave a positive Kaiser test (positive control). The resin was then treated with the following conditions to form the lactam: 2 mL of 0.5N HOBt, 2 mL of 0.5N PyBop and 1 mL of 2N DIEA, twice for 4 hours with constant stirring. A second round of lactam forming conditions was employed: 1 mL 0.5N HOAT, 1 mL of 0.5N HATU and 1 mL of 1N DIEA, for 2 hours with constant stirring. The resin was washed with DMF, followed by 3 washes with DCM.

To the resin, 5 mL of the following reagent was added: 2% triisopropylsilane, 5% water, 5% (w/v) dithiothrieitol, 88% trifluoroacetic acid, and allowed to mix for 3.5 hours. The filtrate was collected into 40 mL of cold anhydrous ethyl ether. The precipitate was pelleted for 10 minutes at 3500 RPM in a refrigerated centrifuge. The ether was decanted, and the peptide re-suspended in fresh ether. The ether workup was performed a total of 3 times. Following the last ether wash the peptide was allowed to air dry to remove residual ether. The peptide was dissolved in 10% acetonitrile and analyzed by mass spectrometry. A single product with a mass of 783.4 Da was identified, which corresponded to the desired lactam-peptide product. The crude product (˜65 mg) was purified on a preparative HPLC equipped with a C18 column using a suitable elution gradient. The fractions were checked by analytical HPLC and those containing pure product were pooled and lyophilized to dryness to give 12.3 mg (17% yield) of a white solid. Purity was greater than 77% based on the analytical HPLC analysis. Electro-spray ionization mass spectrometry (ESI-MS) analysis gave the molecular weight at 783.4 (in agreement with the calculated molecular weight of 782.94).

### Example 6

### Isobutyryl-c(Asp-Lys-Trp-Ile-Gln-Dap)-NH₂(SEQ ID NO:59)

The title peptide was synthesized using Fmoc-chemistry on an Advanced ChemTech Apex peptide synthesizer (Advanced ChemTech, Louisville, Ky., USA). 115 mg of 0.78 mmol/g (0.09 mmol) Rink Amide MBHA resin (Novabiochem, San Diego, Calif., USA) was placed in a reaction well and pre-swollen in 1.5 mL of DMF prior to synthesis. Cycle 1: The resin was treated with two 1.5 mL portions of 25% piperidine in DMF for 5 and 25 minutes, respectively, followed by 4 washes of 1.5 mL DMF. Amino acids stocks were prepared in (NMP) as 0.45N solutions containing 0.45N HOBt. DIC was prepared as a 0.75N solution in NMP. To the resin 1.1 mL of the first amino acid (0.49 mmol), Fmoc-Dap(Mtt)-OH, (Novabiochem, San Diego, Calif., USA) was added along with 0.65 mL (0.49 mmol) of DIC. After one hour of constant mixing the coupling reagents were drained from the resin, and the coupling step repeated. The resin wash washed with two 1.5 mL aliquots of DMF for 1 minute. The process of assembling the peptide (deblock/wash/acylate/wash) was repeated for cycles 2-5 identical to that as described for cycle 1. The following amino acids were used: for cycle 2, Fmoc-Gln(Trt)-OH (Genzyme, Cambridge, Mass., USA); for cycle 3, Fmoc-Ile-OH (Novabiochem, San Diego, Calif., USA); for cycle 4, Fmoc-Trp(Boc)-OH (Genzyme, Cambridge, Mass., USA); for cycle 5, Fmoc-Lys(Boc)-OH (Novabiochem, San Diego, Calif., USA); and for cycle 6, Fmoc-Asp(O-2-PhiPr)-OH, (Novabiochem, San Diego, Calif., USA). The N-terminal Fmoc was removed with 25% piperidine in DMF, followed by four 1.5 mL DMF washes for 1 minute. DIEA, 0.7 mL of 4N in NMP, along with 1.1 mL of 0.45N isobutyric anhydride in NMP was added to the resin and mixed for 30 minutes, and repeated once. The resin was washed with DMF, followed by 3 washes with dichloromethane (DCM).

The side-chain protecting group of Asp(O-2-PhiPr) and Dap(Mtt) were deblocked using ten, 5 mL washes of 5% TIS, 1% TFA in DCM for 2 minutes. Following the last wash the resin was washed with DCM and DMF twice respectively. The resin was treated with 25% piperdine twice for 2 minutes, followed by 4 DMF washes. An aliquot of resin (<1 mg) gave a positive Kaiser test (positive control). The resin was then treated with the following conditions to form the lactam: 2 mL of 0.5N HOBt, 2 mL of 0.5N PyBop and 1 mL of 2N DIEA, twice for 4 hours with constant stirring. A follow-up Kaiser test was still positive indicating incomplete lactam formation. A second round of lactam forming conditions was employed: 1 mL 0.5N HOAT, 1 mL of 0.5N HATU and 1 mL of 1N DIEA, for 2 hours with constant stirring. A follow-up Kaiser test showed no blue color change indicating no presence of a free amine. The resin was washed with DMF, followed by 3 washes with DCM. To the resin, 5 mL of the following reagent was added: 2% triisopropylsilane, 5% water, 5% (w/v) dithiothrieitol, 88% trifluoroacetic acid, and allowed to mix for 3.5 hours. The filtrate was collected into 40 mL of cold anhydrous ethyl ether. The precipitate was pelleted for 10 minutes at 3500 RPM in a refrigerated centrifuge. The ether was decanted, and the peptide re-suspended in fresh ether. The ether workup was performed a total of 3 times. Following the last ether wash, the peptide was allowed to air dry to remove residual ether. The peptide was dissolved in 10% acetonitrile and analyzed by mass spectrometry. A single product with a mass of 826.5 Da was identified, which corresponded to the desired lactam-peptide product. The crude product (˜65 mg) was purified on a preparative HPLC equipped with a C18 column using a suitable elution gradient. The fractions were checked by analytical HPLC and those containing pure product were pooled and lyophilized to dryness to give 4 mg of a white solid. Purity was 89% based on the analytical HPLC analysis. Electro-spray ionization mass spectrometry (ESI-MS) analysis gave the molecular weight at 826.5 (in agreement with the calculated molecular weight of 825.96).

### Example 7

### Isobutyryl-c(Dap-Lys-Trp-Ile-Gln-Glu)-NH₂ (SEQ ID NO:62)

The title peptide was synthesized using Fmoc-chemistry on an Advanced ChemTech Apex peptide synthesizer (Advanced ChemTech, Louisville, Ky., USA). 115 mg of 0.78 mmol/g (0.09 mmol) Rink Amide MBHA resin (Novabiochem, San Diego, Calif., USA) was placed in a reaction well and pre-swollen in 1.5 mL of DMF prior to synthesis. Cycle 1: The resin was treated with two 1.5 mL portions of 25% piperidine in DMF for 5 and 25 minutes, respectively, followed by 4 washes of 1.5 mL DMF. Amino acids stocks were prepared in NMP as 0.45N solutions containing 0.45N HOBt. DIC was prepared as a 0.75N solution in NMP. To the resin 1.1 mL of the first amino acid (0.49 mmol), Fmoc-Glu(O-2-PhiPr)-OH (Novabiochem, San Diego, Calif., USA) was added along with 0.65 mL (0.49 mmol) of DIC. After one hour of constant mixing the coupling reagents were drained from the resin, and the coupling step repeated. Following amino acid acylation the resin was washed with two 1.5 mL aliquots of DMF for 1 minute. The process of assembling the peptide (deblock/wash/acylate/wash) was repeated for cycles 2-5 identical to that as described for cycle 1. The following amino acids were used: for cycle 2, Fmoc-Gln(Trt)-OH (Genzyme, Cambridge, Mass., USA); for cycle 3, Fmoc-Ile-OH (Novabiochem, San Diego, Calif., USA); for cycle 4, Fmoc-Trp(Boc)-OH (Genzyme, Cambridge, Mass., USA); for cycle 5, Fmoc-Lys(Boc)-OH (Novabiochem, San Diego, Calif., USA); for cycle 6, Fmoc-Dap(Mtt)-OH (Novabiochem, San Diego, Calif., USA). The N-terminal Fmoc was removed with 25% piperidine in DMF, followed by four 1.5 mL DMF washes for 1 minute. DIEA, 0.7 ml of 4N in NMP, along with 1.1 mL of 0.45N isobutyric anhydride in NMP was added to the resin and mixed for 30 minutes, and repeated once. The resin was washed with DMF, followed by 3 washes with dichloromethane (DCM). The side-chain protecting group of Glu(O-2-PhiPr) and Dap(Mtt) were deblocked using ten, 5 mL washes of 5% TIS, 1% TFA in DCM for 2 minutes. Following the last wash the resin was washed with DCM and DMF twice respectively. The resin was treated with 25% piperdine twice for 2 minutes, followed by 4 DMF washes. An aliquot of resin (<1 mg) gave a positive Kaiser test (positive control). The resin was then treated with the following conditions to form the lactam: 2 mL of 0.5N HOBt, 2 mL of 0.5N PyBop and 1 mL of 2M DIEA, twice for 4 hours with constant stirring. A follow-up Kaiser test was still positive indicating incomplete lactam formation. A second round of lactam forming conditions was employed: 1 mL 0.5N HOAT, 1 mL of 0.5N HATU and 1 mL of 1N DIEA, for 2 hours with constant stirring. A follow-up Kaiser test showed no blue color change indicating no presence of a free amine. The resin was washed with DMF, followed by 3 washes with DCM.

To the resin, 5 mL of the following reagent was added: 2% triisopropylsilane, 5% water, 5% (w/v) dithiothrieitol, 88% trifluoroacetic acid, and allowed to mix for 3.5 hours. The filtrate was collected into 40 mL of cold anhydrous ethyl ether. The precipitate was pelleted for 10 minutes at 3500 RPM in a refrigerated centrifuge. The ether was decanted, and the peptide re-suspended in fresh ether. The ether workup was performed a total of 3 times. Following the last ether wash the peptide was allowed to air dry to remove residual ether. The peptide was dissolved in 10% acetonitrile and analyzed by mass spectrometry. A single product with a mass of 840.5 Da was identified, which corresponded to the desired lactam-peptide product. The crude product (˜65 mg) was purified on a preparative HPLC equipped with a C18 column using a suitable elution gradient. The fractions were checked by analytical HPLC and those containing pure product were pooled and lyophilized to dryness to give 23 mg of a white solid. Purity was 84% based on the analytical HPLC analysis. Electro-spray ionization mass spectrometry (ESI-MS) analysis gave the molecular weight at 840.5 (in agreement with the calculated molecular weight of 839.99).

### Example 8

### c(Glutaryl-Lys-Trp-Ile-Gln-Orn)-NH₂ (SEQ ID NO:67)

The title peptide was synthesized using Fmoc-chemistry on an Advanced ChemTech Apex peptide synthesizer (Advanced ChemTech, Louisville, Ky., USA). 115 mg of 0.78 mmol/g (0.09 mmol) Rink Amide MBHA resin (Novabiochem, San Diego, Calif., USA) was placed in a reaction well and pre-swollen in 1.5 mL of DMF prior to synthesis. Cycle 1: The resin was treated with two 1.5 mL portions of 25% piperidine in DMF for 5 and 25 minutes, respectively, followed by 4 washes of 1.5 mL DMF. Amino acids stocks were prepared in (NMP) as 0.45N solutions containing 0.45N HOBt. DIC was prepared as a 0.75N solution in NMP. To the resin 1.1 mL of the first amino acid (0.49 mmol), Fmoc-Orn(Mtt)-OH (Novabiochem, San Diego, Calif., USA) was added along with 0.65 mL (0.49 mmol) of DIC. After one hour of constant mixing the coupling reagents were drained from the resin, and the coupling step repeated. The resin wash washed with two 1.5 mL aliquots of DMF for 1 minute. The process of assembling the peptide (deblock/wash/acylate/wash) was repeated for cycles 2-5 identical to that as described for cycle 1. The following amino acids were used: for cycle 2, Fmoc-Gln(Trt)-OH (Genzyme, Cambridge, Mass., USA); for cycle 3, Fmoc-Ile-OH (Novabiochem, San Diego, Calif., USA); for cycle 4, Fmoc-Trp(Boc)-OH (Genzyme, Cambridge, Mass., USA); and for cycle 5, Fmoc-Lys(Boc)-OH (Novabiochem, San Diego, Calif., USA). The N-terminal Fmoc was removed with 25% piperidine in DMF, followed by four 1.5 mL DMF washes for 1 minute. DIEA, 0.7 mL of 4N in NMP, along with 1.1 mL of 0.45N glutaric anhydride in NMP was added to the resin and mixed for 30 minutes, and repeated once. The resin was washed with DMF, followed by 3 washes with dichloromethane (DCM).

The side-chain protecting group of Orn(Mtt) was deblocked using ten, 5 mL washes of 5% TIS, 1% TFA in DCM for 2 minutes. Following the last wash the resin was washed with DCM and DMF twice respectively. The resin was treated with 25% piperdine twice for 2 minutes, followed by 4 DMF washes. An aliquot of resin (<1 mg) gave a positive Kaiser test (positive control). The resin was then treated with the following conditions to form the lactam: 2 mL of 0.5N HOBt, 2 mL of 0.5N PyBop and 1 mL of 2N DIEA, twice for 4 hours with constant stirring. A follow-up Kaiser test was still positive indicating incomplete lactam formation. A second round of lactam forming conditions was employed: 1 mL 0.5N HOAT, 1 mL of 0.5N HATU and 1 mL of IN DIEA, for 2 hours with constant stirring. A follow-up Kaiser test showed no blue color change indicating no presence of a free amine. The resin was washed with DMF, followed by 3 washes with DCM. To the resin, 5 mL of the following reagent was added: 2% triisopropylsilane, 5% water, 5% (w/v) dithiothrieitol, 88% trifluoroacetic acid, and allowed to mix for 3.5 hours. The filtrate was collected into 40 mL of cold anhydrous ethyl ether. The precipitate was pelleted for 10 minutes at 3500 RPM in a refrigerated centrifuge. The ether was decanted, and the peptide re-suspended in fresh ether. The ether workup was performed a total of 3 times. Following the last ether wash the peptide was allowed to air dry to remove residual ether. The peptide was dissolved in 10% acetonitrile and analyzed by mass spectrometry. A single product with a mass of 783.6 Da was identified, which corresponded to the desired lactam-peptide product. The crude product (˜65 mg) was purified on a preparative HPLC equipped with a C18 column using a suitable elution gradient. The fractions were checked by analytical HPLC and those containing pure product were pooled and lyophilized to dryness to give 12 mg of a white solid. Purity was 99% based on the analytical HPLC analysis. Electro-spray ionization mass spectrometry (ESI-MS) analysis gave the molecular weight at 783.6 (in agreement with the calculated molecular weight of 782.93).

The following examples were made according to the appropriate procedures described hereinabove:

(SEQ ID NO: 1)
Example 9:
Ac-c(Cys-Leu-Asp-Pro-Lys-Gln-Lys-Trp-Ile-Gln-Cys)-
NH₂
(SEQ ID NO: 2)
Example 10:
Leu-c(Cys-Asp-Pro-Lys-Gln-Lys-Trp-Ile-Gln-Cys)-NH₂
(SEQ ID NO: 3)
Example 11:
Leu-Asp-c(Cys-Pro-Lys-Gln-Lys-Trp-Ile-Gln-Cys)-NH₂
(SEQ ID NO: 4)
Example 12:
Leu-Asp-Pro-Lys-Gln-c(Cys-Lys-Trp-Ile-Gln-Cys)-NH₂
(SEQ ID NO: 5)
Example 13:
Leu-Asp-Pro-Lys-Gln-Lys-c(Cys-Trp-Ile-Gln-Cys)-NH₂
(SEQ ID NO: 6)
Example 14:
Leu-Asp-Pro-Lys-Gln-Lys-Trp-c(Cys-Ile-Gln-Cys)-NH₂
(SEQ ID NO: 7)
Example 15:
Lys-Trp-c(Cys-Ile-Gln-Cys)-NH₂
(SEQ ID NO: 8)
Example 16:
Ac-c(Cys-Leu-Asp-Pro-Lys-Gln-Lys-Trp-Ile-Gln-Cys)-
D-Dap-NH₂
(SEQ ID NO: 9)
Example 17:
Ac-Asp-c(Cys-Leu-Asp-Pro-Lys-Gln-Lys-Trp-Ile-Gln-
Cys)-D-Dap-NH₂
(SEQ ID NO: 8)
Example 18:
Ac-c(Cys-Leu-Asp-Pro-Lys-Gln-Lys-Trp-Ile-Gln-Cys)-
Dab-NH₂
(SEQ ID NO: 2)
Example 19:
Ac-Leu-c(Cys-Asp-Pro-Lys-Gln-Lys-Trp-Ile-Gln-Cys)-
NH₂
(SEQ ID NO: 3)
Example 20:
Ac-Leu-Asp-c(Cys-Pro-Lys-Gln-Lys-Trp-Ile-Gln-Cys)-
NH₂
(SEQ ID NO: 10)
Example 21:
Ac-Leu-Asp-Pro-c(Cys-Lys-Gln-Lys-Trp-Ile-Gln-Cys)-
NH₂
(SEQ ID NO: 11)
Example 22:
Ac-Leu-Asp-Pro-Lys-c(Cys-Gln-Lys-Trp-Ile-Gln-Cys)-
NH₂
(SEQ ID NO: 4)
Example 23:
Ac-Leu-Asp-Pro-Lys-Gln-c(Cys-Lys-Trp-Ile-Gln-Cys)-
NH₂
(SEQ ID NO: 12)
Example 24:
Ac-c(Cys-Leu-Asp-Pro-Lys-Gln-Lys-Trp-Ile-D-Gln-
Cys)-NH₂
(SEQ ID NO: 13)
Example 25:
Ac-c(Cys-Leu-Asp-Pro-Lys-Gln-Lys-Trp-D-Ile-Gln-
Cys)-NH₂
(SEQ ID NO: 14)
Example 26:
Ac-c(Cys-Leu-Asp-Pro-Lys-Gln-Lys-D-Trp-Ile-Gln-
Cys)-NH₂
(SEQ ID NO: 15)
Example 27:
Ac-c(Cys-Leu-Asp-Pro-Lys-Gln-D-Lys-Trp-Ile-Gln-
Cys)-NH₂
(SEQ ID NO: 16)
Example 28:
Ac-c(Cys-Leu-Asp-Pro-Lys-D-Gln-Lys-Trp-Ile-Gln-
Cys)-NH₂
(SEQ ID NO: 17)
Example 29:
Ac-c(Cys-Leu-Asp-Pro-D-Lys-Gln-Lys-Trp-Ile-Gln-
Cys)-NH₂
(SEQ ID NO: 18)
Example 30:
Ac-c(Cys-Leu-Asp-D-Pro-Lys-Gln-Lys-Trp-Ile-Gln-
Cys)-NH₂
(SEQ ID NO: 19)
Example 31:
Ac-c(Cys-Leu-D-Asp-Pro-Lys-Gln-Lys-Trp-Ile-Gln-
Cys)-NH₂
(SEQ ID NO: 20)
Example 32:
Ac-c(Cys-D-Leu-Asp-Pro-Lys-Gln-Lys-Trp-Ile-Gln-
Cys)-NH₂
(SEQ ID NO: 1)
Example 33:
c(Cys-Leu-Asp-Pro-Lys-Gln-Lys-Trp-Ile-Gln-Cys)-
NH₂
(SEQ ID NO: 21)
Example 34:
Ac-Lys-c(Cys-Leu-Asp-Pro-Lys-Gln-Lys-Trp-Ile-Gln-
Cys)-NH₂
(SEQ ID NO: 22)
Example 35:
c(D-Cys-Leu-Asp-Pro-Lys-Gln-Lys-Trp-Ile-Gln-Cys)-
Dap-NH₂
(SEQ ID NO: 23)
Example 36:
c(Cys-Leu-Asp-Pro-Lys-Gln-Lys-Trp-Ile-Gln-D-Cys)-
NH₂
(SEQ ID NO: 24)
Example 37:
c(D-Cys-Leu-Asp-Pro-Lys-Gln-Lys-Trp-Ile-Gln-D-
Cys)-NH₂
(SEQ ID NO: 10)
Example 38:
Leu-Asp-Pro-c(Cys-Lys-Gln-Lys-Trp-Ile-Gln-Cys)-
NH₂
(SEQ ID NO: 25)
Example 39:
c(D-Cys-Leu-Asp-Pro-Lys-Gln-Lys-Trp-Ile-Gln-Cys)-
NH₂
(SEQ ID NO: 26)
Example 40:
Asp-Pro-c(Cys-Lys-Gln-Lys-Trp-Ile-Gln-Cys)-NH₂
(SEQ ID NO: 27)
Example 41:
Asp-Pro-Lys-c(Cys-Gln-Lys-Trp-Ile-Gln-Cys)-NH₂
(SEQ ID NO: 28)
Example 42:
c(Cys-Asp-Pro-Lys-Gln-Lys-Trp-Ile-Gln-Cys)-NH₂
(SEQ ID NO: 29)
Example 43:
Pro-c(Cys-Lys-Gln-Lys-Trp-Ile-Gln-Cys)-NH₂
(SEQ ID NO: 30)
Example 44:
Pro-Lys-c(Cys-Gln-Lys-Trp-Ile-Gln-Cys)-NH₂
(SEQ ID NO: 31)
Example 45:
Pro-Lys-Gln-c(Cys-Lys-Trp-Ile-Gln-Cys)-NH₂
(SEQ ID NO: 32)
Example 46:
c(Cys-Pro-Lys-Gln-Lys-Trp-Ile-Gln-Cys)-NH₂
(SEQ ID NO: 33)
Example 47:
Lys-c(Cys-Gln-Lys-Trp-Ile-Gln-Cys)-NH₂
(SEQ ID NO: 34)
Example 48:
Lys-Gln-c(Cys-Lys-Trp-Ile-Gln-Cys)-NH₂
(SEQ ID NO: 35)
Example 49:
c(Cys-Lys-Gln-Lys-Trp-Ile-Gln-Cys)-NH₂
(SEQ ID NO: 36)
Example 50:
c(Cys-Gln-Lys-Trp-Ile-Gln-Cys)-NH₂
(SEQ ID NO: 37)
Example 51:
Lys-c(Cys-Trp-Ile-Gln-Cys)-NH₂
(SEQ ID NO: 39)
Example 52:
pGlu-c(Cys-Lys-Trp-Ile-Gln-Cys)-NH₂
(SEQ ID NO: 39)
Example 53:
DpGlu-c(Cys-Lys-Trp-Ile-Gln-Cys)-NH₂
(SEQ ID NO: 39)
Example 54:
Ac-Gln-c(Cys-Lys-Trp-Ile-Gln-Cys)-NH₂
(SEQ ID NO: 38)
Example 55:
Ac-c(Cys-Lys-Trp-Ile-Gln-Cys)-NH₂
(SEQ ID NO: 38)
Example 56:
nButyryl-c(Cys-Lys-Trp-Ile-Gln-Cys)-NH₂
(SEQ ID NO: 38)
Example 57:
Benzoyl-c(Cys-Lys-Trp-Ile-Gln-Cys)-NH₂
(SEQ ID NO: 38)
Example 58:
Isobutyryl-c(Cys-Lys-Trp-Ile-Gln-Cys)-NH₂
(SEQ ID NO: 40)
Example 59:
c((βA1a-Lys-Trp-Gln-Asp)-NH₂
(SEQ ID NO: 41)
Example 60:
c(Gaba-Lys-Trp-Ile-Gln-Asp)-NH₂
(SEQ ID NO: 42)
Example 61:
c(Gaba-Lys-Trp-Ile-Gln-Glu)-NH₂
(SEQ ID NO: 43)
Example 62:
c(Succinyl-Lys-Trp-Ile-Gln-Orn)-NH₂
(SEQ ID NO: 44)
Example 63:
c(Succinyl-Lys-Trp-Ile-Gln-Lys)-NH₂
(SEQ ID NO: 45)
Example 64:
Ac-c(Asp-Lys-Trp-Ile-Gln-Lys)-NH₂
(SEQ ID NO: 46)
Example 65:
c(Ac-Lys-Trp-Ile-Gln-Cys)-NH₂
(SEQ ID NO: 38)
Example 66:
Trimethylacetyl-c(Cys-Lys-Trp-Ile-Gln-Cys)-NH₂
(SEQ ID NO: 38)
Example 67:
4-Biphenylcarboxyl-c(Cys-Lys-Trp-Ile-Gln-Cys)-NH₂
(SEQ ID NO: 38)
Example 68:
1-Naphthoyl-c(Cys-Lys-Trp-Ile-Gln-Cys)-NH₂
(SEQ ID NO: 38)
Example 69:
2-Naphthoyl-c(Cys-Lys-Trp-Ile-Gln-Cys)-NH₂
(SEQ ID NO: 38)
Example 70:
2-Indolecarboxyl-c(Cys-Lys-Trp-Ile-Gln-Cys)-NH₂
(SEQ ID NO: 38)
Example 71:
p-Tolylacetyl-c(Cys-Lys-Trp-Ile-Gln-Cys)-NH₂
(SEQ ID NO: 38)
Example 72:
trans-Ciimamoyl-c(Cys-Lys-Trp-Ile-Gln-Cys)-NH₂
(SEQ ID NO: 38)
Example 73:
4-Chlorocinnamoyl-c(Cys-Lys-Trp-Ile-Gln-Cys)-NH₂
(SEQ ID NO: 48)
Example 74:
Ac-c(Cys-Lys-Trp-Ile-Gln-D-Cys)-NH₂
(SEQ ID NO: 49)
Example 75:
Ac-c(Cys-Lys-Trp-Ile-D-Gln-Cys)-NH₂
(SEQ ID NO: 50)
Example 76:
Ac-c(Cys-Lys-Trp-D-Ile-Gln-Cys)-NH₂
(SEQ ID NO: 51)
Example 77:
Ac-c(Cys-Lys-D-Trp-Ile-Gln-Cys)-NH₂
(SEQ ID NO: 52)
Example 78:
Ac-c(Cys-D-Lys-Trp-Ile-Gln-Cys)-NH₂
(SEQ ID NO: 53)
Example 79:
Ac-c(D-Cys-Lys-Trp-Ile-Gln-Cys)-NH₂
(SEQ ID NO: 54)
Example 80:
(Des-amino-4-methyl)Phe-c(Cys-Lys-Trp-Ile-Gln-
Cys)-NH₂
(SEQ ID NO: 38)
Example 81:
n-Hexanoyl-c(Cys-Lys-Trp-Ile-Gln-Cys)-NH₂
(SEQ ID NO: 38)
Example 82:
Octanoyl-c(Cys-Lys-Trp-Ile-Gln-Cys)-NH₂
(SEQ ID NO: 38)
Example 83:
Decanoyl-c(Cys-Lys-Trp-Ile-Gln-Cys)-NH₂
(SEQ ID NO: 38)
Example 84:
Dodecanoyl-c(Cys-Lys-Trp-Ile-Gln-Cys)-NH₂
(SEQ ID NO: 55)
Example 85:
Trp-c(Cys-Ile-Gln-Cys)-NH₂
(SEQ ID NO: 56)
Example 86:
c(Cys-Ile-Gln-Cys)-NH₂
(SEQ ID NO: 58)
Example 87:
c(Gly-Lys-Trp-Ile-Gln-Asp)-NH₂
(SEQ ID NO: 58)
Example 88:
c(Gly-Lys-Trp-Ile-Gln-Glu)-NH₂
(SEQ ID NO: 42)
Example 89:
c((βAla-Lys-Trp-Ile-Gln-Glu)-NH₂
(SEQ ID NO: 41)
Example 90:
c(Apn-Lys-Trp-Ile-Gln-Asp)-NH₂
(SEQ ID NO: 42)
Example 91:
c(Apn-Lys-Trp-Ile-Gln-Glu)-NH₂
(SEQ ID NO: 42)
Example 92:
c(Ahx-Lys-Trp-Ile-Gln-Glu)-NH₂
(SEQ ID NO: 41)
Example 93:
c(Aha-Lys-Trp-Ile-Gln-Asp)-NH₂
(SEQ ID NO: 42)
Example 94:
c(Aha-Lys-Trp-Ile-Gln-Glu)-NH₂
(SEQ ID NO: 41)
Example 95:
c(Aoc-Lys-Trp-Ile-Gln-Asp)-NH₂
(SEQ ID NO: 42)
Example 96:
c(Aoc-Lys-Trp-Ile-Gln-Glu)-NH₂
(SEQ ID NO: 41)
Example 97:
c(Anc-Lys-Trp-Ile-Gln-Asp)-NH₂
(SEQ ID NO: 42)
Example 98:
c(Anc-Lys-Trp-Ile-Gln-Glu)-NH₂
(SEQ ID NO: 59)
Example 99:
Isobutyryl-c(Asp-Lys-Trp-Ile-Gln-Orn)-NH₂
(SEQ ID NO: 60)
Example 100:
Isobutyryl-c(Glu-Lys-Trp-Ile-Gln-Dap)-NH₂
(SEQ ID NO: 60)
Example 101:
Isobutyryl-c(Glu-Lys-Trp-Ile-Gln-Dab)-NH₂
(SEQ ID NO: 60)
Example 102:
Isobutyryl-c(Glu-Lys-Trp-Ile-Gln-Orn)-NH₂
(SEQ ID NO: 60)
Example 103:
Isobutyryl-c(Glu-Lys-Trp-Ile-Gln-Lys)-NH₂
(SEQ ID NO: 61)
Example 104:
Isobutyryl-c(Dap-Lys-Trp-Ile-Gln-Asp)-NH₂
(SEQ ID NO: 61)
Example 105:
Isobutyryl-c(Dab-Lys-Trp-Ile-Gln-Asp)-NH₂
(SEQ ID NO: 61)
Example 106:
Isobutyryl-c(Lys-Lys-Trp-Ile-Gln-Asp)-NH₂
(SEQ ID NO: 62)
Example 107:
Isobutyryl-c(Dab-Lys-Trp-Ile-Gln-Glu)-NH₂
(SEQ ID NO: 62)
Example 108:
Isobutyryl-c(Orn-Lys-Trp-Ile-Gln-Glu)-NH₂
(SEQ ID NO: 62)
Example 109:
Isobutyryl-c(Lys-Lys-Trp-Ile-Gln-Glu)-NH₂
(SEQ ID NO: 63)
Example 110:
Ac-c(Asp-Lys-Trp-Ile-Gln-Dap)-NH₂
(SEQ ID NO: 63)
Example 111:
Ac-c(Asp-Lys-Trp-Ile-Gln-Dab)-NH₂
(SEQ ID NO: 63)
Example 112:
Ac-c(Asp-Lys-Trp-Ile-Gln-Orn)-NH₂
(SEQ ID NO: 64)
Example 113:
Ac-c(Glu-Lys-Trp-Ile-Gln-Dap)-NH₂
(SEQ ID NO: 64)
Example 114:
Ac-c(Glu-Lys-Trp-Ile-Gln-Dab)-NH₂
(SEQ ID NO: 64)
Example 115:
Ac-c(Glu-Lys-Trp-Ile-Gln-Orn)-NH₂
(SEQ ID NO: 64)
Example 116:
Ac-c(Glu-Lys-Trp-Ile-Gln-Lys)-NH₂
(SEQ ID NO: 65)
Example 117:
Ac-c(Dap-Lys-Trp-Ile-Gln-Asp)-NH₂
(SEQ ID NO: 65)
Example 118:
Ac-c(Dab-Lys-Trp-Ile-Gln-Asp)-NH₂
(SEQ ID NO: 65)
Example 119:
Ac-c(Orn-Lys-Trp-Ile-Gln-Asp)-NH₂
(SEQ ID NO: 65)
Example 120:
Ac-c(Lys-Lys-Trp-Ile-Gln-Asp)-NH₂
(SEQ ID NO: 66)
Example 121:
Ac-c(Dap-Lys-Trp-Ile-Gln-Glu)-NH₂
(SEQ ID NO: 66)
Example 122:
Ac-c(Dab-Lys-Trp-Ile-Gln-Glu)-NH₂
(SEQ ID NO: 66)
Example 123:
Ac-c(Orn-Lys-Trp-Ile-Gln-Glu)-NH₂
(SEQ ID NO: 66)
Example 124:
Ac-c(Lys-Lys-Trp-Ile-Gln-Glu)-NH₂
(SEQ ID NO: 43)
Example 125:
c(Succinyl-Lys-Trp-Ile-Gln-Dap)-NH₂
(SEQ ID NO: 43)
Example 126:
c(Succinyl-Lys-Trp-Ile-Gln-Dab)-NH₂
(SEQ ID NO: 67)
Example 127:
c(Glutaryl-Lys-Trp-Ile-Gln-Dap)-NH₂NH₂
(SEQ ID NO: 67)
Example 128:
c(Glutaryl-Lys-Trp-Ile-Gln-Dab)-NH₂
(SEQ ID NO: 67)
Example 129:
c(Glutaryl-Lys-Trp-Ile-Gln-Lys)-NH₂
(SEQ ID NO: 68)
Example 130:
c(Diglycolyl-Lys-Trp-Ile-Gln-Dap)-NH₂
(SEQ ID NO: 68)
Example 131:
c(Diglycolyl-Lys-Trp-Ile-Gln-Dab)-NH₂
(SEQ ID NO: 68)
Example 132:
c(Diglycolyl-Lys-Trp-Ile-Gln-Orn)-NH₂
(SEQ ID NO: 68)
Example 133:
c(Diglycolyl-Lys-Trp-Ile-Gln-Lys)-NH₂
(SEQ ID NO: 69)
Example 134:
c(Adipoyl-Lys-Trp-Ile-Gln-Dap)-NH₂
(SEQ ID NO: 69)
Example 135:
c(Adipoyl-Lys-Trp-Ile-Gln-Dab)-NH₂
(SEQ ID NO: 69)
Example 136:
c(Adipoyl-Lys-Trp-Ile-Gln-Orn)-NH₂
(SEQ ID NO: 69)
Example 137:
c(Adipoyl-Lys-Trp-Ile-Gln-Lys)-NH₂
(SEQ ID NO: 70)
Example 138:
c(D-Cys-D-Gln-D-Ile-D-Trp-D-Lys-D-Gln-D-Lys-D-Pro-
D-Asp-D-Leu-D-Cys)-Doc-Aoc-OH
(SEQ ID NO: 71)
Example 139:
c(D-Cys-D-Gln-D-Ile-D-Trp-D-Lys-D-Gln-D-Lys-D-Pro-
D-Asp-D-Leu-D-Cys)-Aoc-OH
(SEQ ID NO: 72)
Example 140:
c(D-Cys-D-Gln-D-Ile-D-Trp-D-Lys-D-Gln-D-Lys-D-Pro-
D-Asp-D-Leu-D-Cys)-Ile-Glu-Aoc-OH
(SEQ ID NO: 73)
Example 141:
c(D-Cys-D-Gln-D-Ile-D-Trp-D-Lys-D-Gln-D-Lys-D-Pro-
D-Asp-D-Leu-D-Cys)-D-Val-D-Glu-Aoc-OH
(SEQ ID NO: 71)
Example 142:
c(D-Cys-D-Gln-D-Ile-D-Trp-D-Lys-D-Gln-D-Lys-D-Pro-
D-Asp-D-Leu-D-Cys)-Gaba-OH
(SEQ ID NO: 71)
Example 143:
c(D-Cys-D-Gln-D-Ile-D-Trp-D-Lys-D-Gln-D-Lys-D-Pro-
D-Asp-D-Leu-D-Cys)-Ahx-OH
(SEQ ID NO: 70)
Example 144:
c(D-Cys-D-Gln-D-Ile-D-Trp-D-Lys-D-Gln-D-Lys-D-Pro-
D-Asp-D-Leu-D-Cys)-Asp-Aoc-OH
(SEQ ID NO: 70)
Example 145:
c(D-Cys-D-Gln-D-Ile-D-Trp-D-Lys-D-Gln-D-Lys-D-Pro-
D-Asp-D-Leu-D-Cys)-Glu-Aoc-OH
(SEQ ID NO: 74)
Example 146:
c(D-Cys-D-Gln-D-Ile-D-Trp-D-Lys-D-Gln-D-Lys-D-Pro-
D-Asp-D-Leu-D-Cys)-D-Lys(N/-octanoyl)-OH
(SEQ ID NO: 75)
Example 147:
c(D-Cys-D-Gln-D-Ile-D-Trp-D-Lys-D-Gln-D-Lys-D-Pro-
D-Asp-D-Leu-D-Cys)-D-Asp-Lys(Nε-octanoyl)-OH
(SEQ ID NO: 75)
Example 148:
c(D-Cys-D-Gln-D-Ile-D-Trp-D-Lys-D-Gln-D-Lys-D-Pro-
D-Asp-D-Leu-D-Cys)-Asp-Lys(Nε-octanoyl)-OH
(SEQ ID NO: 75)
Example 149:
c(D-Cys-D-Gln-D-Ile-D-Trp-D-Lys-D-Gln-D-Lys-D-Pro-
D-Asp-D-Leu-D-Cys)-Lys(Nε-octanoyl)-OH
(SEQ ID NO: 76)
Example 150:
c(D-Cys-D-Gln-D-Ile-D-Trp-D-Lys-D-Gln-D-Lys-D-Pro-
D-Asp-D-Leu-D-Cys)-D-Val-D-Glu-Lys(Nε-octanoyl)-OH
(SEQ ID NO: 75)
Example 151:
c(D-Cys-D-Gln-D-Ile-D-Trp-D-Lys-D-Gln-D-Lys-D-Pro-
D-Asp-D-Leu-D-Cys)-Doc-Lys(Nε-octanoyl)-OH
(SEQ ID NO: 77)
Example 152:
HEPA-c(D-Cys-D-Gln-D-Ile-D-Trp-D-Lys-D-Gln-D-Lys-
D-Pro-D-Asp-D-Leu-D-Cys)-NH₂
(SEQ ID NO: 78)
Example 153:
c(D-Cys-D-Gln-D-Ile-D-Trp-D-Lys-D-Gln-D-Lys-D-Pro-
D-Asp-D-Leu-Lys(Nε-octanoyl)-D-Cys)-NH2
(SEQ ID NO: 79)
Example 154:
c(D-Cys-D-Gln-D-Ile-D-Trp-D-Lys-D-Gln-D-Lys-D-Pro-
D-Asp-Lys(Nε-octanoyl)-D-Leu-D-Cys)-OH
(SEQ ID NO: 13)
Example 155:
Ac-c(Cys-Leu-Asp-Pro-Lys-Gln-Lys-Trp-A6c-Gln-Cys)-
NH₂
(SEQ ID NO: 80)
Example 156:
Ac-c(Cys-A6c-Asp-Pro-Lys-Gln-Lys-Trp-Gln-Cys)-NH₂
(SEQ ID NO: 81)
Example 157:
Ac-c(Cys-Lys-Gln-Lys-Trp-Ile-Gln-Cys)-D-Pro-D-Asp-
D-Leu-NH₂
(SEQ ID NO: 82)
Example 158:
Ac-c(Cys-Pro-Lys-Gln-Lys-Trp-Ile-Gln-Cys)-D-Asp-D-
Leu-NH₂
(SEQ ID NO: 83)
Example 159:
c(D-Cys-D-Gln-D-Ile-D-Trp-D-Lys-D-Gln-D-Lys-D-Pro-
D-Asp(D-Ala-OH)-D-Leu-D-Cys)-NH₂
(SEQ ID NO: 84)
Example 160:
Octanoyl-Dap-c(D-Cys-D-Gln-D-Ile-D-Trp-D-Lys-D-
Gln-D-Lys-D-Pro-D-Asp-D-Leu-D-Cys)-NH₂
(SEQ ID NO: 111)
Example 161:
c(D-Cys-D-Gln-D-Ile-D-Trp-D-Lys-D-Gln-D-Lys-D-
Pro-D-Asp(1-hexanol)-D-Leu-D-Cys)-NH₂
(SEQ ID NO: 85)
Example 162:
Ac-Lys(N/-dodecanoyl)-c(Cys-Leu-Asp-Pro-Lys-Gln-
Lys-Trp-Ile-Gln-Cys)-NH₂
(SEQ ID NO: 86)
Example 163:
BOC-Trp-Gly-α-aminoglutaramide
(SEQ ID NO: 86)
Example 164:
BOC-Trp-Val-α-aminoglutaramide
(SEQ ID NO: 86)
Example 165:
BOC-Trp-βA1a-α-aminoglutaramide
(SEQ ID NO: 87)
Example 166:
D-Lys(Nε-tetradecanoyl)-D-Asp-c(Cys-Leu-Asp-Pro-
Lys-Gln-Lys-Trp-Ile-Gln-Cys)-NH₂
(SEQ ID NO: 88)
Example 167:
D-Lys-D-Asp-c(Cys-Leu-Asp-Pro-Lys-Gln-Lys-Trp-Ile-
Gln-Cys)-NH₂
(SEQ ID NO: 88)
Example 168:
D-Lys(Nε-octanoyl)-D-Asp-c(Cys-Leu-Asp-Pro-Lys-
Gln-Lys-Trp-Ile-Gln-Cys)-NH₂
(SEQ ID NO: 89)
Example 169:
Lys(Nε-dodecanoyl)-Ile-c(Cys-Leu-Asp-Pro-Lys-Gln-
Lys-Trp-Ile-Gln-Cys)-NH₂
(SEQ ID NO: 89)
Example 170:
Lys(Nε-dodecanoyl)-Asp-c(Cys-Leu-Asp-Pro-Lys-Gln-
Lys-Trp-Ile-Gln-Cys)-NH₂
(SEQ ID NO: 90)
Example 171:
BOC-Tcc-βAla-α-aminoglutaramide
(SEQ ID NO: 91)
Example 172:
D-Lys(Nε-dodecanoyl)-Asp-c(Cys-Leu-Asp-Pro-Lys-
Gln-Lys-Trp-Ile-Gln-Cys)-NH₂
(SEQ ID NO: 92)
Example 173:
Lys{Nε-[γ-Glu-(Nα-decanoyl)]}-c(Cys-Leu-Asp-Pro-
Lys-Gln-Lys-Trp-Ile-Gln-Cys)-NH₂
(SEQ ID NO: 90)
Example 174:
BOC-Tic-βAla-α-aminoglutaramide
(SEQ ID NO: 93)
Example 175:
3-indolepropionyl-L-valyl-α-aminoglutaramide
(SEQ ID NO: 94)
Example 176:
N(3-indolylpropionyl)-βAla-α-aminoglutarimide
(SEQ ID NO: 95)
Example 177:
Des-amino-Trp-βAla[ψ(CH₂NH)]-α-aminoglutarimide
(SEQ ID NO: 94)
Example 178:
N-(3-indolepropyl)-βAla-α-aminoglutarimide
(SEQ ID NO: 96)
Example 179:
N-(isopropoxycarbonyl)-Trp-βAla-α-aminoglutarimide
(SEQ ID NO: 97)
Example 180:
Trp-Gly-α-aminoglutaramide
(SEQ ID NO: 97)
Example 181:
Trp-Val-α-aminoglutaramide
(SEQ ID NO: 97)
Example 182:
Trp-βAla-α-aminoglutaramide
(SEQ ID NO: 98)
Example 183:
c(D-Cys-D-Gln-D-Ile-D-Trp-D-Lys-D-Cys)-NH₂
(SEQ ID NO: 99)
Example 184:
BOC-Trp-βhVal-α-aminoglutarimide
(SEQ ID NO: 100)
Example 185:
Tryptaminylcarbonyl-βhVal-α-aminoglutarimide
(SEQ ID NO: 101)
Example 186:
[aminoPeg₁₀-oxaglutaryl]₂-c(D-Cys-D-Gln-D-Ile-D-
Trp-D-Lys-D-Gln-D-Lys-D-Pro-D-Asp-D-Leu-D-Cys)-NH₂
(SEQ ID NO: 101)
Example 187:
[aminoPeg₁₀-oxaglutaryl]-c(D-Cys-D-Gln-D-Ile-D-
Trp-D-Lys-D-Gln-D-Lys-D-Pro-D-Asp-D-Leu-D-Cys)-NH₂
(SEQ ID NO: 102)
Example 188:
Trp-Aib-aminoglutarimide
(SEQ ID NO: 103)
Example 189:
BOC-tryptophanolyl-carbonyl-Val-aminoglutarimide
(SEQ ID NO: 98)
Example 190:
Ac-c(D-Cys-D-Gln-D-Ile-D-Trp-D-Lys-D-Cys)-NH₂
(SEQ ID NO: 98)
Example 191:
nButyryl-c(D-Cys-D-Gln-D-Ile-D-Trp-D-Lys-D-Cys)-
NH₂
(SEQ ID NO: 98)
Example 192:
Benzoyl-c(D-Cys-D-Gln-D-Ile-D-Trp-D-Lys-D-Cys)-NH₂
(SEQ ID NO: 98)
Example 193:
Isobutyryl-c(D-Cys-D-Gln-D-Ile-D-Trp-D-Lys-D-Cys)-
NH₂
SEQ ID NO: 104)
Example 194:
Amino-tryptophanolyl-carbonyl-Val-aminoglutarimide
(SEQ ID NO: 105)
Example 195:
c((βAla-D-Gln-D-Ile-D-Trp-D-Lys-D-Asp)-NH₂
(SEQ ID NO: 105)
Example 196:
c(Gaba-D-Gln-D-Ile-D-Trp-D-Lys-D-Asp)-NH₂
(SEQ ID NO: 105)
Example 197:
c(Apn-D-Gln-D-Ile-D-Trp-D-Lys-D-Asp)-NH₂
(SEQ ID NO: 106)
Example 198:
Ac-c(D-Cys-D-Gln-D-Ile-D-Trp-D-Lys-D-Gln-D-Lys-D-
Pro-D-Asp-D-Leu-D-Cys)-NH₂
(SEQ ID NO: 107)
Example 199:
c(βAla-D-Gln-D-Ile-D-Trp-D-Lys-D-Gln-D-Lys-D-Pro-
D-Asp-D-Leu-D-Asp)-NH₂
(SEQ ID NO: 108)
Example 200:
Butanyl-c(D-Cys-D-Gln-D-Val-D-Trp-D-Lys-D-Cys)-NH₂
(SEQ ID NO: 109)
Example 201:
Isobutyryl-Asp-Lys-Trp-Ile-Gln-Lys-NH₂
(SEQ ID NO: 110)
Example 202:
Isobutyryl-Orn-Lys-Trp-Ile-Gln-Asp-NH₂

Physical data for the compounds exemplified herein are given in Table 1.

TABLE 1

| Example | Calculated | Experimental | |
|---|---|---|---|
| Number | Mol. Wt. | Mol. Wt. | Purity |
| 9 | 1400.68 | 1401.2 | 99 |
| 10 | 1358.65 | 1358.4 | 92 |
| 11 | 1358.65 | 1358 | 96 |
| 12 | 1358.65 | 1358.2 | 86 |
| 13 | 1358.65 | 1358.4 | 87 |
| 14 | 1358.65 | 1358.2 | 88 |
| 15 | 776.98 | 776.4 | 90 |
| 16 | 1486.78 | 1486 | 99 |
| 17 | 1601.86 | 1600.8 | 97.7 |
| 18 | 1500.8 | 1500.2 | 98 |
| 19 | 1400.68 | 1400.4 | 93.2 |
| 20 | 1400.68 | 1399.8 | 95.4 |
| 21 | 1400.68 | 1400.2 | 78.9 |
| 22 | 1400.68 | 1399.8 | 86.5 |
| 23 | 1400.68 | 1399.8 | 82.8 |
| 24 | 1400.68 | 1400.2 | 86.5 |
| 25 | 1400.68 | 1400.4 | 94.3 |
| 26 | 1400.68 | 1400.2 | 88.2 |
| 27 | 1400.68 | 1400.2 | 89.6 |
| 28 | 1400.68 | 1400 | 90 |
| 29 | 1400.68 | 1400.2 | 79.1 |
| 30 | 1400.68 | 1400 | 88.8 |
| 31 | 1400.68 | 1400.2 | 92.1 |
| 32 | 1400.68 | 1400.2 | 85.2 |
| 33 | 1358.65 | 1357 | 98 |
| 34 | 1528.86 | 1528.8 | 94 |
| 35 | 1444.74 | N/A | 96.6 |
| 36 | 1358.65 | N/A | 86.2 |
| 37 | 1358.65 | N/A | 94.1 |
| 38 | 1358.65 | N/A | 90.2 |
| 39 | 1358.65 | N/A | 95.9 |
| 40 | 1245.49 | 1244.28 | 95 |
| 41 | 1245.49 | N/A | 99 |
| 42 | 1245.49 | N/A | 96.6 |
| 43 | 1130.4 | N/A | 97.6 |
| 44 | 1130.4 | N/a | 97.3 |
| 45 | 1130.4 | N/A | 95.8 |
| 46 | 1130.4 | N/A | 93.5 |
| 47 | 1033.28 | N/A | 97.6 |
| 48 | 1033.28 | 1032.33 | 95 |
| 49 | 1033.28 | N/A | 98 |
| 50 | 905.11 | N/A | 94.4 |
| 51 | 776.98 | N/A | 96.5 |
| 52 | 888.079 | N/A | 89.5 |
| 53 | 888.079 | 887.5 | 93.5 |
| 54 | 947.147 | 945.6 | 81.9 |
| 55 | 819.017 | 818.3 | 94.3 |
| 56 | 847.07 | N/A | 88.1 |
| 57 | 881.087 | N/A | 88.3 |
| 58 | 847.07 | 847.5 | 93.9 |
| 59 | 627.699 | 627.1 | 99 |
| 60 | 754.885 | 754.1 | 96 |
| 61 | 768.911 | 768.5 | 84 |
| 62 | 768.911 | 767.8 | 97 |
| 63 | 782.938 | 782.4 | 76 |
| 64 | 839.99 | 839 | 99 |
| 65 | 715.872 | 715.3 | 97 |
| 66 | 861.097 | 860.80 | 93 |
| 67 | 957.185 | 956.7 | 95 |
| 68 | 931.147 | 930.50 | 99 |
| 69 | 931.147 | 930.5 | 95 |
| 70 | 920.124 | 919.36 | 96.2 |
| 71 | 909.141 | 908.7 | 95 |
| 72 | 907.125 | 906.8 | 95 |
| 73 | 941.57 | 940.60 | 96 |
| 74 | 819.017 | 818.2 | 91 |
| 75 | 819.017 | 818.1 | 99 |
| 76 | 819.017 | 818.1 | 99 |
| 77 | 819.017 | 818.1 | 97 |
| 78 | 819.017 | 818.1 | 99 |
| 79 | 819.017 | 818.2 | 99 |
| 80 | 923.168 | 922.4 | 99.9 |
| 81 | 875.124 | 874.4 | 98 |
| 82 | 903.177 | 902.7 | 92 |
| 83 | 931.231 | 930.60 | 94 |
| 84 | 959.285 | 958.6 | 99.9 |
| 85 | 648.806 | 647.9 | 92 |
| 86 | 462.593 | 461.6 | 96 |
| 87 | 726.831 | 726.4 | 90.8 |
| 88 | 740.858 | 740.5 | 98.3 |
| 89 | 754.885 | 754.6 | 95.9 |
| 90 | 768.911 | 768.6 | 91.4 |
| 91 | 782.938 | 782.7 | 95.4 |
| 92 | 796.965 | 796.6 | 94.4 |
| 93 | 796.965 | 796.6 | 93.9 |
| 94 | 810.992 | 810.6 | 90 |
| 95 | 810.992 | 810.6 | 80.8 |
| 96 | 825.019 | 824.8 | 91.8 |
| 97 | 825.019 | 824.5 | 96.4 |
| 98 | 839.045 | 839.6 | 92 |
| 99 | 854.017 | 853.8 | 95.1 |
| 100 | 839.99 | 839.8 | 93.2 |
| 101 | 854.017 | 853.2 | 65.8 |
| 102 | 868.044 | 867.8 | 88.3 |
| 103 | 882.07 | 881.6 | 83.9 |
| 104 | 825.963 | 825.6 | 95.9 |
| 105 | 839.99 | 839.5 | 83.3 |
| 106 | 868.044 | 867.6 | 77.5 |
| 107 | 854.017 | 853.6 | 82.4 |
| 108 | 868.044 | 867.7 | 87.4 |
| 109 | 882.07 | 881.7 | 96.2 |
| 110 | 797.909 | 797.5 | 98.7 |
| 111 | 811.936 | 811.3 | 99.9 |
| 112 | 825.963 | 823.52 | 97.3 |
| 113 | 811.936 | 811.3 | 97.2 |
| 114 | 825.963 | 825.3 | 98.2 |
| 115 | 839.99 | 839.4 | 99.2 |
| 116 | 854.017 | 853.4 | 97.3 |
| 117 | 797.909 | 979.3 | 94.8 |
| 118 | 811.936 | 811.3 | 98.8 |
| 119 | 825.963 | 825.3 | 97.4 |
| 120 | 839.99 | 839.4 | 99.9 |
| 121 | 811.936 | 811.3 | 96.4 |
| 122 | 825.963 | 825.6 | 90 |
| 123 | 839.99 | 839.4 | 96.2 |
| 124 | 854.017 | 853.4 | 74.5 |
| 125 | 740.858 | 740.6 | 97.2 |
| 126 | 754.885 | 754.6 | 99.9 |
| 127 | 754.885 | 754.6 | 99.6 |
| 128 | 768.911 | 768.6 | 96.6 |
| 129 | 796.965 | 796.3 | 99 |
| 130 | 756.857 | 756.6 | 88.9 |
| 131 | 770.884 | 770.6 | 99.9 |
| 132 | 784.91 | 784.3 | 99.1 |
| 133 | 798.937 | 798.3 | 99.9 |
| 134 | 768.911 | 768.3 | 99.9 |
| 135 | 782.938 | 782.3 | 99.9 |
| 136 | 796.965 | 796.4 | 99.9 |
| 137 | 810.992 | 810.4 | 99.9 |
| 138 | 1646 | 1645.8 | 94 |
| 139 | 1500.84 | 1500.4 | 99 |
| 140 | 1743.12 | 1743 | 99 |
| 141 | 1729.09 | 1728.4 | 99 |
| 142 | 1444.74 | 1444.6 | 98 |
| 143 | 1472.79 | 1472.4 | 94 |
| 144 | 1615.93 | 1615.7 | 99 |
| 145 | 1629.96 | 1629.8 | 99 |
| 146 | 1614 | 1613.8 | 99 |
| 147 | 1729.09 | 1729 | 99 |
| 148 | 1729.09 | 1728.3 | 99 |
| 149 | 1614 | 1613.4 | 99 |
| 150 | 1842.25 | 1841.4 | 99 |
| 151 | 1759.16 | 1758.8 | 99 |
| 152 | 1528.86 | N/A | 99 |
| 153 | 1613.02 | 1612.2 | 99 |
| 154 | 1614 | 1612.4 | 99 |
| 155 | 1412.69 | 1411.6 | 95.2 |
| 156 | 1299.54 | 1298.4 | 94 |
| 157 | 1400.68 | 1400.4 | 82.1 |
| 158 | 1400.68 | 1400 | 90.5 |
| 159 | 1429.72 | 1429.5 | 99 |
| 160 | 1570.94 | 1570.1 | 99 |
| 161 | 1442.81 | 1442.5 | 99 |
| 162 | 1711.16 | 1710 | 99 |
| 163 | 471.511 | 470.6 | 90 |
| 164 | 513.591 | 513.2 | 90 |
| 165 | 485.538 | 484.5 | 90 |
| 166 | 1812.27 | 1811 | 85 |
| 167 | 1601.91 | 1600.4 | 94 |
| 168 | 1728.11 | 1726.8 | 92 |
| 169 | 1782.28 | 1781 | 99 |
| 170 | 1784.21 | 1783.3 | 97 |
| 171 | 497.549 | 496.7 | 90 |
| 172 | 1784.21 | 1782.3 | 93 |
| 173 | 1770.19 | 1770.2 | 94 |
| 174 | 458.512 | 457.6 | 90 |
| 175 | 398.46 | 398.3 | 96 |
| 176 | 370.407 | 370.1 | 94 |
| 177 | 356.424 | 356.1 | 97 |
| 178 | 356.424 | 356.1 | 97 |
| 179 | 471.511 | 471.1 | 97 |
| 180 | 371.395 | 370.6 | 89 |
| 181 | 413.475 | 412.7 | 94 |
| 182 | 385.422 | 384.7 | 94 |
| 183 | 776.98 | 775.8 | 99 |
| 184 | 527.618 | 527.3 | 90 |
| 185 | 427.502 | 427.2 | 90 |
| 186 | 2556.01 | 2556.1 | 95 |
| 187 | 1957.33 | 1956.9 | 95 |
| 188 | 399.448 | 399.3 | 99 |
| 189 | 543.617 | N/A | 99 |
| 190 | 819.017 | N/A | 92.3 |
| 191 | 847.07 | 846.4 | 95.5 |
| 192 | 881.087 | N/A | 91.2 |
| 193 | 847.07 | N/A | 81.4 |
| 194 | 443.501 | 443.2 | 99.9 |
| 195 | 740.858 | 740.3 | 88 |
| 196 | 754.885 | 754.4 | 98 |
| 197 | 768.911 | 768.5 | 99 |
| 198 | 1400.68 | 1399.6 | 99 |
| 199 | 1322.52 | 1321.6 | 96 |
| 200 | 833.043 | 832.2 | 93 |
| 201 | 886.058 | 885.8 | 80.3 |
| 202 | 872.031 | 871.7 | 99 |

A selection of the exemplified compounds were tested using the above-discussed assay methods and other assay methods well know to those skilled in the relevant art and the results are reported in Table 2.

TABLE 2

| | | | T ½ | | T ½ |
|---|---|---|---|---|---|
| Example | v MCP-1 | v IL-8 | rat plasma | | rat |
| Number | EC50 (nM) | inhibition (%) | in vitro (hr) | | in vivo (min) |
| 1 | 12 | 86.7 | >28 | h | 19 |
| 2 | 7 | | | | |
| 3 | 6 | | | | |
| 4 | 20 | | | | |
| 5 | 5 | | | | |
| 6 | 5 | | | | |
| 7 | 5 | | | | |
| 8 | 5 | | | | |
| 9 | 2 | 102.3 | | | 5.4 |
| 10 | 10 | 102.2 | | | |
| 11 | 330 | 38.1 | | | |
| 12 | 50 | 94.8 | | | |
| 13 | 8000 | 5.8 | | | |
| 14 | 2000 | 10.5 | | | |
| 15 | 3000 | 10.5 | | | |
| 16 | 800 | 19.8 | | | |
| 17 | >1000 | 15.5 | | | |
| 18 | 20 | 103.5 | | | |
| 19 | 7 | 100.5 | | | <<5 |
| 20 | 8 | 93.9 | | | 2.7/4.7 |
| 21 | 7 | 96.3 | | | 22 |
| 22 | 900 | 16.8 | | | |
| 23 | >1000 | 13.4 | | | |
| 24 | 200 | 33.8 | | | |
| 25 | 30 | 96.9 | | | |
| 26 | 20 | 94.9 | | | |
| 27 | 7 | 91.0 | | | 22.7/2,.3 |
| 28 | 4 | 96.9 | | | 14 |
| 29 | 30 | 92.0 | | | |
| 30 | 8 | 99.2 | | | <10 |
| 31 | 50 | 95.8 | | | |
| 32 | >1000 | 8.7 | | | |
| 33 | 20 | 90.0 | >15 | | 6 |
| 34 | 250 | 75.7 | | | |
| 35 | 50 | 91.9 | | | |
| 36 | 600 | 21.8 | | | |
| 37 | >1000 | −14.7 | | | |
| 38 | >1000 | −12 | | | |
| 39 | >1000 | 3.8 | | | |
| 40 | 7 | 113.5 | | | |
| 41 | 80 | 67.8 | | | |
| 42 | 25 | 94.4 | | | |
| 43 | 600 | 21.9 | | | |
| 44 | 1000 | 12.9 | | | |
| 45 | 150 | 74.2 | | | |
| 46 | 150 | 52.4 | | | |
| 47 | 20 | 103.2 | | | |
| 48 | 8 | 91.9 | | | <5 |
| 49 | 100 | 72.6 | | | |
| 50 | 25 | 104.8 | | | |
| 51 | 80 | 115.9 | | | |
| 52 | 25 | | | | |
| 53 | 6 | | | | 19.4/23.1 |
| 54 | 10 | | | | 30.3/20.9 |
| 55 | 6 | | | | 18.0/16.5 |
| 56 | 25 | | | | |
| 57 | 15 | | | | |
| 58 | 8 | | | | |
| 59 | 25 | | | | |
| 60 | 30 | | | | |
| 61 | 25 | | | | |
| 62 | 66 | | | | |
| 63 | 66 | | | | |
| 64 | 20 | | | | |
| 65 | 30 | | | | |
| 66 | 10 | | | | |
| 67 | 8 | | | | |
| 68 | 30 | | | | |
| 69 | 15 | | | | |
| 70 | 5 | | | | |
| 71 | 10 | | | | |
| 72 | 8 | | | | |
| 73 | 10 | | | | |
| 74 | 7 | | | | |
| 75 | 25 | | | | |
| 76 | 20 | | | | |
| 77 | 20 | | | | |
| 78 | 30 | | | | |
| 79 | 60 | | | | |
| 80 | 10 | | | | |
| 81 | 5 | | | | |
| 82 | 25 | | | | |
| 83 | 20 | | | | |
| 84 | 15 | | | | |
| 85 | 50 | | | | |
| 86 | 7.5 | | | | |
| 87 | 15 | | | | |
| 88 | 9 | | | | |
| 89 | 20 | | | | |
| 90 | 7 | | | | |
| 91 | 9 | | | | |
| 92 | 5 | | | | |
| 93 | 8 | | | | |
| 94 | 10 | | | | |
| 95 | 20 | | | | |
| 96 | 4 | | | | |
| 97 | 15 | | | | |
| 98 | 15 | | | | |
| 99 | 50 | | | | |
| 100 | 10 | | | | |
| 101 | 4 | | | | |
| 102 | 10 | | | | |
| 103 | 8 | | | | |
| 104 | 50 | | | | |
| 105 | 15 | | | | |
| 106 | 8 | | | | |
| 107 | 8 | | | | |
| 108 | 8 | | | | |
| 109 | 8 | | | | |
| 110 | 8 | | | | |
| 111 | 4 | | | | |
| 112 | 6 | | | | |
| 113 | 30 | | | | |
| 114 | 8 | | | | |
| 115 | 5 | | | | |
| 116 | 8 | | | | |
| 117 | 5 | | | | |
| 118 | 8 | | | | |
| 119 | 8 | | | | |
| 120 | 8 | | | | |
| 121 | 10 | | | | |
| 122 | 10 | | | | |
| 123 | 7 | | | | |
| 124 | 8 | | | | |
| 125 | 15 | | | | |
| 126 | 6 | | | | |
| 127 | 7 | | | | |
| 128 | 10 | | | | |
| 129 | 400 | | | | |
| 130 | 50 | | | | |
| 131 | 30 | | | | |
| 132 | 200 | | | | |
| 138 | 20 | 98.4 | | | |
| 139 | 100 | 47.0 | | | |
| 140 | 400 | 43.4 | | | |
| 141 | 330 | 27.5 | | | |
| 142 | 20 | 107.3 | | | |
| 143 | 30 | 103.5 | | | |
| 144 | 3000 | 25.4 | | | |
| 145 | 200 | 23.8 | | | |
| 146 | 800 | 46.3 | | | |
| 147 | 500 | 29.0 | | | |
| 148 | 6 | 100.9 | | | 39 |
| 149 | 66 | 106.2 | | | |
| 150 | 33 | 100.5 | | | |
| 151 | 28 | 103.7 | | | |
| 152 | 20 | 103.5 | | | 38 |
| 153 | >1000 | 4.1 | | | |
| 154 | >1000 | 9.8 | | | |
| 155 | >1000 | −1.5 | | | |
| 156 | >1000 | 3.1 | | | |
| 157 | >1000 | −15 | | | |
| 158 | >1000 | −17.9 | | | |
| 160 | 25 | 100.2 | | | |
| 161 | 8 | 99.8 | | | |
| 162 | 20 | 101.1 | | | 17 |
| 163 | 20 | 105.7 | 6.1 | | |
| 164 | 20 | 104.4 | 11.1 | | 33 |
| 165 | 5 | 111.5 | 4.0 | | |
| 166 | 25 | 100.7 | | | |
| 167 | 25 | 99.6 | | | |
| 168 | 600 | 21.3 | | | |
| 169 | >1000 | 7.7 | | | |
| 170 | >1000 | −5.1 | | | 5 |
| 171 | 602 | 84.6 | 8.5 | | |
| 172 | 333 | | | | |
| 173 | >1000 | | | | |
| 174 | 200 | | | | |
| 175 | 15 | | 6.8 | | 16.0/15.8 |
| 176 | 200 | | 5.4 | | |
| 177 | >1000 | | 4.0 | | |
| 178 | 50 | | 1.7 | | |
| 179 | | | 9.1 | | |
| 180 | 25 | | ND | | |
| 181 | 20 | | ND | | |
| 182 | 6 | | 0.9 | | |
| 183 | 20 | | >24 | | |
| 184 | 10 | | 1.3 | | |
| 185 | 15 | | 2.6 | | |
| 186 | 20 | | | | |
| 187 | 66 | | | | |
| 188 | 66 | | 3.1 | | |
| 189 | ND | | | | |
| 190 | 30 | | | | |
| 191 | 5 | | | | 30 |
| 192 | 7 | | | | |
| 193 | 25 | | | | |
| 194 | 50 | | | | |
| 195 | 45 | | | | |
| 196 | 50 | | | | |
| 197 | 20 | | | | |
| 198 | 20 | | | | |
| 199 | 20 | | | | |
| 200 | 30 | | | | |
| 201 | 10 | | | | |
| 202 | 8 | | | | |

Dosages, Formulations and Routes of Administration of the Agents of the Invention

The therapeutic agents of the invention, including their salts, are preferably administered so as to achieve serum levels of about 0.01 pM to about 100 nM, more preferably at doses of about 0.01 pM to about 5nM, and even more preferably at doses of about 0.1 pM to about 2 nM, of the therapeutic agent. To achieve these levels, the agent may be administered at dosages of at least 0.01 to about 100 mg/kg, more preferably about 0.1 to about 50 mg/kg, and even more preferably about 0.1 to about 30 mg/kg, of body weight, although other dosages may provide beneficial results. The amount administered will vary depending on various factors including, but not limited to, the agent chosen, the disease, whether prevention or treatment is to be achieved, and if the agent is modified for bioavailability and in vivo stability.

One or more suitable unit dosage forms comprising the therapeutic agents of the invention, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipients to form a pharmaceutical formulation, or unit dosage form. The total active ingredients in such formulation comprise from 0.1 to 99.9% by weight of the formulation. By “pharmaceutically acceptable” it is meant the carrier, diluent, excipients, and/or salt must be compatible with the other ingredients of the formulation, and not deleterious to the recipient thereof The active ingredient for oral administration may be present as a powder or as granules; as a solution, a suspension or an emulsion; or in achievable base as a synthetic resin for ingestion of the active ingredients from a chewing gum. The active ingredient may also be presented as a bolus, electuary or paste.

Formulations suitable for vaginal administration may be presented as pessaries, tampons, creams, gels, pastes, douches, lubricants, foams or sprays containing, in addition to the active ingredient, such carriers as are known in the art to be appropriate. Formulations suitable for rectal administration may be presented as suppositories.

Pharmaceutical formulations containing the therapeutic agents of the invention can be prepared by procedures known in the art using well known and readily available ingredients. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturing agents such as glycerol; disintegrating agents such as calcium carbonate and sodium bicarbonate; agents for retarding dissolution such as paraffin; resorption accelerators such as quarternary ammonium compounds; surface active agents such as acetyl alcohol, glycerol monostearate; adsorptive carriers such as kaolin and bentonite; and lubricants such as talc, calcium and magnesium stearate, and solid polyethyl glycols.

For example, tablets and caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration and may be presented in unit dose form in ampules, pre-filled syringes, small volume infusion containers or in multi-dose containers with an added preservative.

These formulations can contain pharmaceutically acceptable vehicles and adjuvants which are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

Further, the compound of this invention can be administered in a sustained release composition such as those described in the following patents and patent applications. U.S. Pat. No. 5,672,659 teaches sustained release compositions comprising a bioactive agent and a polyester. U.S. Pat. No. 5,595,760 teaches sustained release compositions comprising a bioactive agent in a gelable form. U.S. Pat. No. 5,821,221 teaches polymeric sustained release compositions comprising a bioactive agent and chitosan. U.S. Pat. No. 5,916,883 teaches sustained release compositions comprising a bioactive agent and cyclodextrin. The teachings of the foregoing patents and applications are incorporated herein by reference.

The use of immediate or of sustained release compositions depends on the type of indications targeted. If the indication consists of an acute or over-acute disorder, a treatment with an immediate form will be preferred over the same with a prolonged release composition. On the contrary, for preventive or long-term treatments, a prolonged release composition will generally be preferred.

The therapeutic agents of the invention can be delivered via patches for transdermal administration. See U.S. Pat. No. 5,560,922 for examples of patches suitable for transdermal delivery of a therapeutic agent.

For administration to the upper (nasal) or lower respiratory tract by inhalation, the therapeutic agents of the invention are conveniently delivered form an insufflator, nebulizer or pressurized pack or other convenient means of delivering an aerosol spray.

The local delivery of the therapeutic agents of the invention can also be by a variety of techniques which administer the agent at or near the site of disease. Examples include local delivery catheters, shunts and stents or other implantable devices, site specific carriers, direct injection, or direct applications.

For topical administration, the therapeutic agents may be formulated as is known in the art for direct application to a target area. Conventional forms for this purpose include would dressings, coated bandages or other polymer covering, ointments, creams, lotions, pastes, jellies, sprays, and aerosols, as well as in toothpaste and mouthwash, or by other suitable forms, e.g., via a coated condom. The active ingredients can also be delivered via iontophoresis, e.g., as disclosed in U.S. Pat. Nos. 4,140,122; 4,383,529; or 4,051,842.

The formulations and compositions described herein may also contain other ingredients such as antimicrobial agents, or preservatives. Furthermore, the active ingredients may also be used in combination with other therapeutic agents, for example, oral contraceptives, bronchodilators, anti-viral agents, steroids and the like.

It is to be understood that while the invention has been described in conjunction with the detailed description thereof, that the foregoing description is intended to illustrate and not to limit the scope of the invention. Other aspects, advantages, and modifications are within the claims. Also, the contents of each references cited herein is incorporated by reference in its entirety.

What is claimed is:

1. A peptide of formula (I): 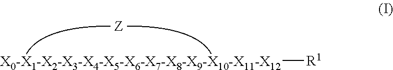
wherein
X₀ is H, pGlu, D-pGlu, (R²,R³)Y₀-Y₁-Y₂-Y₃-Y₄-Y₅-Y₆-Y₇-, or deleted;
X₁ is Cys, hCys, Pen, Lys, Orn, Dab, Dap, Glu, Asp, D-Cys, D-hCys, D-Pen, D-Lys, D-Orn, D-Dab, D-Dap, D-Glu, D-Asp —HN—(CR⁵R⁶)ₙ—C(O)—, —C(O)—(CR⁷R⁸)ₘ—(O)ₓ—) (CR⁹C¹⁰)ₛ—C(O)—, or —CH₂—(CR¹¹C¹²)ₜ—C(O)—;
X₂ is Leu, hLeu, Ile, Cha, Val, Abu, D-Leu, D-hLeu, D-Ile, D-Cha, D-Val, D-Abu, or deleted;
X₃ is Aps, Glu, D-Asp, D-Glu, or deleted;
X₄ is Pro, Dmt, hPro, Thz, 3Hyp, 4Hyp, D-Pro, D-Dmt, D-hPro, D-Thi, D-Thz, D-3Hyp, D-4Hyp, or deleted;
X₅ is Lys, Arg, hArg, Orn, Dab, Dap, D-Lys, D-Arg, D-hArg, D-Orn, D-Dab, D-Dap, or deleted;
X₆ is Gln, Asn, D-Gln, D-Asn, or deleted;
X₇ is Lys, Arg, hArg, Orn, Dab, Dap, D-Lys, D-Arg, D-hArg, D-Orn, D-Dab, D-Dap, or deleted;
X₈ is Trp, 1Nal, 2Nal, D-Trp, D-1Nal, D-2Nal, or deleted;
X₉ is Ile, Leu, hLeu, Cha, Val, Abu, D-Ile, D-Leu, D-hLeu, D-Cha, D-Val, or D-Abu;
X₁₀ is Gln, Asn, D-Gln, or D-Asn;
X₁₁ is Cys, hCys, Pen, Lys, Orn, Dab, Dap, Glu, Asp, D-Cys, D-hCys, D-Pen, D-Lys, D-Orn, D-Dab, D-Dap, D-Glu, or D-Asp;
X₁₂ is Lys, Arg, hArg, Orn, Dab, Dap, D-Lys, D-Arg, D-hArg, D-Orn, D-Dab, D-Dap, or deleted;
Y₀ is Aps, Glu, D-Asp, D-Glu, or deleted;
Y₁ is Leu, hLeu, Ile, Cha, Val, Abu, D-Leu, D-hLeu, D-Ile, D-Cha, D-Val, D-Abu, or deleted;
Y₂ is Aps, Glu, D-Asp, D-Glu, or deleted;
Y₃ is Pro, Dmt, hPro, Thz, 3Hyp, 4Hyp, D-Pro, D-Dmt, D-hPro, D-Thi, D-Thz, D-3Hyp, D-4Hyp, or deleted;
Y₄ is Lys, Arg, hArg, Orn, Dab, Dap, D-Lys, D-Arg, D-hArg, D-Orn, D-Dab, D-Dap, or deleted;
Y₅ is Gln, Asn, D-Gln, D-Asn, or deleted;
Y₆ is Lys, Arg, hArg, Orn, Dab, Dap, D-Lys, D-Arg, D-hArg, D-Orn, D-Dab, D-Dap, or deleted;
Y₇ is Tip, 1Nal, 2Nal, D-Trp, D-1Nal, D-2Nal, or deleted;
R¹ is —OH or —NH₂;
provided that:
when X₁ and X₁₁ each indenpendently is Cys, hCys, Pen, D-Cys, D-hCys, or D-Pen, Z is a single covalent bond between —S— in the side-chain of X₁ and —S— in the side-chain of X₁₁;
when X₁ is Lys, Orn, Dab, Dap, D-Lys, D-Orn, D-Dab, D-Dap, or —HN—(CR⁵R⁶)ₙ—C(O)— and X₁₁ is Glu, Aps, D-Glu, or D-Aps, Z is an amide bond;
when X₁ is Glu, Asp, D-Glu, D-Asp, or —C(O)—(CR⁷R⁸)ₘ—(O)ₓ—(CR⁹C¹⁰)ₛ—C(O)—, and X₁₁ is Lys, Orn, Dab, Dap, D-Lys, D-Orn, D-Dab, or D-Dap, Z is an amide bond; and
when X₁ is —CH₂—(CR¹¹C¹²)ₜ—C(O)— and X₁₁ is Cys, hCys, Pen, D-Cys, D-hCys, or D-Pen, Z is a single covalent bond between —CH₂— in the side chain of X₁ and —S— of X₁₁;
further provided that:
X₂ and Y₁ do not coexist;
X₃ and Y₂ do not coexist;
X₄ and Y₃ do not coexist;
X₅ and Y₄ do not coexist;
X₆ and Y₅ do not coexist;
X₇ and Y₆ do not coexist;
X₈ and Y₇ do not coexist; and
one of X₈ and Y₇ is not deleted;
each of R² and R³ is, independently for each occurrence, H, (C₁-C₃₀)alkyl, (C₁-C₃₀)acyl, substituted (C₁-C₃₀)alkyl, substituted (C₁-C₃₀)acyl, arylacyl, or arylakylacyl;
further provided that:
when R² is (C₁-C₃₀)acyl, substituted (C₁-C₃₀)acyl, arylacyl, or arylakylacyl, then R³ is H, (C₁-C₃₀)alkyl, or substituted (C₁-C₃₀)alkyl;
each of R⁵, R⁶, R⁷, R⁸, R⁹, R₁₁, and R¹² is, independently for each occurrence, H, halogen, or (C₁-C₁₀)alkyl;
R⁵ and R⁶ can joint together to form a ring system;
R⁷ and R⁸ can joint together to form a ring system;
R⁹ and R¹⁰ can joint together to form a ring system;
R¹¹ and R¹² can joint together to form a ring system;
n is 1-20;
m is 1-5;
s is 1-5;
x is 0 or 1; and
t is 1-5.

2. A compound according to claim 1 wherein said compound is
(SEQ ID NO: 1)
Ac-c(Cys-Leu-Asp-Pro-Lys-Gln-Lys-Trp-Ile-Gln-Cys)-
NH₂,
(SEQ ID NO: 2)
Leu-c(Cys-Asp-Pro-Lys-Gln-Lys-Trp-Ile-Gln-Cys)-
NH₂,
(SEQ ID NO: 3)
Leu-Asp-c(Cys-Pro-Lys-Gln-Lys-Trp-Ile-Gln-Cys)-
NH₂,
(SEQ ID NO: 4)
Leu-Asp-Pro-Lys-Gln-c(Cys-Lys-Trp-Ile-Gln-Cys)-
NH₂,
(SEQ ID NO: 5)
Leu-Asp-Pro-Lys-Gln-Lys-c(Cys-Trp-Ile-Gln-Cys)-
NH₂,
(SEQ ID NO: 6)
Leu-Asp-Pro-Lys-Gln-Lys-Trp-c(Cys-Ile-Gln-Cys)-
NH₂,
(SEQ ID NO: 7)
Lys-Trp-c(Cys-Ile-Gln-Cys)-NH₂,
(SEQ ID NO: 8)
Ac-c(Cys-Leu-Asp-Pro-Lys-Gln-Lys-Trp-Ile-Gln-Cys)-
D-Dap-NH₂,
(SEQ ID NO: 9)
Ac-Asp-c(Cys-Leu-Asp-Pro-Lys-Gln-Lys-Trp-Ile-Gln-
Cys)-D-Dap-NH₂,
(SEQ ID NO: 8)
Ac-c(Cys-Leu-Asp-Pro-Lys-Gln-Lys-Trp-Ile-Gln-Cys)-
Dab-NH₂,
(SEQ ID NO: 2)
Ac-Leu-c(Cys-Asp-Pro-Lys-Gln-Lys-Trp-Ile-Gln-Cys)-
NH₂,
(SEQ ID NO: 3)
Ac-Leu-Asp-c(Cys-Pro-Lys-Gln-Lys-Trp-Ile-Gln-Cys)-
NH₂,
(SEQ ID NO: 10)
Ac-Leu-Asp-Pro-c(Cys-Lys-Gln-Lys-Trp-Ile-Gln-Cys)-
NH₂,
(SEQ ID NO: 11)
Ac-Leu-Asp-Pro-Lys-c(Cys-Gln-Lys-Trp-Ile-Gln-Cys)-
NH₂,
(SEQ ID NO: 4)
Ac-Leu-Asp-Pro-Lys-Gln-c(Cys-Lys-Trp-Ile-Gln-Cys)-
NH₂,
(SEQ ID NO: 12)
Ac-c(Cys-Leu-Asp-Pro-Lys-Gln-Lys-Trp-Ile-D-Gln-
Cys)-NH₂,
(SEQ ID NO: 13)
Ac-c(Cys-Leu-Asp-Pro-Lys-Gln-Lys-Trp-D-Ile-Gln-
Cys)-NH₂,
(SEQ ID NO: 14)
Ac-c(Cys-Leu-Asp-Pro-Lys-Gln-Lys-D-Trp-Ile-Gln-
Cys)-NH₂,
(SEQ ID NO: 15)
Ac-c(Cys-Leu-Asp-Pro-Lys-Gln-D-Lys-Trp-Ile-Gln-
Cys)-NH₂,
(SEQ ID NO: 16)
Ac-c(Cys-Leu-Asp-Pro-Lys-D-Gln-Lys-Trp-Ile-Gln-
Cys)-NH₂,
(SEQ ID NO: 17)
Ac-c(Cys-Leu-Asp-Pro-D-Lys-Gln-Lys-Trp-Ile-Gln-
Cys)-NH₂,
(SEQ ID NO: 18)
Ac-c(Cys-Leu-Asp-D-Pro-Lys-Gln-Lys-Trp-Ile-Gln-
Cys)-NH₂,
(SEQ ID NO: 19)
Ac-c(Cys-Leu-D-Asp-Pro-Lys-Gln-Lys-Trp-Ile-Gln-
Cys)-NH₂,
(SEQ ID NO: 20)
Ac-c(Cys-D-Leu-Asp-Pro-Lys-Gln-Lys-Trp-Ile-Gln-
Cys)-NH₂,
(SEQ ID NO: 1)
c(Cys-Leu-Asp-Pro-Lys-Gln-Lys-Trp-Ile-Gln-Cys)-
NH₂,
(SEQ ID NO: 21)
Ac-Lys-c(Cys-Leu-Asp-Pro-Lys-Gln-Lys-Trp-Ile-Gln-
Cys)-NH₂,
(SEQ ID NO: 22)
c(D-Cys-Leu-Asp-Pro-Lys-Gln-Lys-Trp-Ile-Gln-Cys)-
Dap-NH₂,
(SEQ ID NO: 23)
c(Cys-Leu-Asp-Pro-Lys-Gln-Lys-Trp-Ile-Gln-D-Cys)-
NH₂,
(SEQ ID NO: 24)
c(D-Cys-Leu-Asp-Pro-Lys-Gln-Lys-Trp-Ile-Gln-D-
Cys)-NH₂,
(SEQ ID NO: 10)
Leu-Asp-Pro-c(Cys-Lys-Gln-Lys-Trp-Ile-Gln-Cys)-
NH₂,
(SEQ ID NO: 25)
c(D-Cys-Leu-Asp-Pro-Lys-Gln-Lys-Trp-Ile-Gln-Cys)-
NH₂,
(SEQ ID NO: 26)
Asp-Pro-c(Cys-Lys-Gln-Lys-Trp-Ile-Gln-Cys)-NH₂,
(SEQ ID NO: 27)
Asp-Pro-Lys-c(Cys-Gln-Lys-Trp-Ile-Gln-Cys)-NH₂,
(SEQ ID NO: 28)
c(Cys-Asp-Pro-Lys-Gln-Lys-Trp-Ile-Gln-Cys)-NH₂,
(SEQ ID NO: 29)
Pro-c(Cys-Lys-Gln-Lys-Trp-Ile-Gln-Cys)-NH₂,
(SEQ ID NO: 30)
Pro-Lys-c(Cys-Gln-Lys-Trp-Ile-Gln-Cys)-NH₂,
(SEQ ID NO: 31)
Pro-Lys-Gln-c(Cys-Lys-Trp-Ile-Gln-Cys)-NH₂,
(SEQ ID NO: 32)
c(Cys-Pro-Lys-Gln-Lys-Trp-Ile-Gln-Cys)-NH₂,
(SEQ ID NO: 33)
Lys-c(Cys-Gln-Lys-Trp-Ile-Gln-Cys)-NH₂,
(SEQ ID NO: 34)
Lys-Gln-c(Cys-Lys-Trp-Ile-Gln-Cys)-NH₂,
(SEQ ID NO: 35)
c(Cys-Lys-Gln-Lys-Trp-Ile-Gln-Cys)-NH₂,
(SEQ ID NO: 36)
c(Cys-Gln-Lys-Trp-Ile-Gln-Cys)-NH₂,
(SEQ ID NO: 37)
Lys-c(Cys-Trp-Ile-Gln-Cys)-NH₂,
(SEQ ID NO: 38)
c(Cys-Lys-Trp-Ile-Gln-Cys)-NH₂,
(SEQ ID NO: 39)
pGlu-c(Cys-Lys-Trp-Ile-Gln-Cys)-NH₂,
(SEQ ID NO: 39)
DpGlu-c(Cys-Lys-Trp-Ile-Gln-Cys)-NH₂,
(SEQ ID NO: 39)
Ac-Gln-c(Cys-Lys-Trp-Ile-Gln-Cys)-NH₂,
(SEQ ID NO: 39)
Ac-D-Gln-c(Cys-Lys-Trp-Ile-Gln-Cys)-NH₂,
(SEQ ID NO: 38)
Ac-c(Cys-Lys-Trp-Ile-Gln-Cys)-NH2,
(SEQ ID NO: 38)
nButyryl-c(Cys-Lys-Trp-Ile-Gln-Cys)-NH₂,
(SEQ ID NO: 38)
Benzoyl-c(Cys-Lys-Trp-Ile-Gln-Cys)-NH₂,
(SEQ ID NO: 38)
Isobutyryl-c(Cys-Lys-Trp-Ile-Gln-Cys)-NH₂,
(SEQ ID NO: 40)
c(βAla-Lys-Trp-Gln-Asp)-NH₂,
(SEQ ID NO: 41)
c(Gaba-Lys-Trp-Ile-Gln-Asp)-NH₂,
(SEQ ID NO: 42)
c(Gaba-Lys-Trp-Ile-Gln-Glu)-NH₂,
(SEQ ID NO: 43)
c(Succinyl-Lys-Trp-Ile-Gln-Orn)-NH₂,
(SEQ ID NO: 44)
c(Succinyl-Lys-Trp-Ile-Gln-Lys)-NH₂,
(SEQ ID NO: 45)
Ac-c(Asp-Lys-Trp-Ile-Gln-Lys)-NH₂,
(SEQ ID NO: 46)
c(Ac-Lys-Trp-Ile-Gln-Cys)-NH₂,
(SEQ ID NO: 38)
Trimethylacetyl-c(Cys-Lys-Trp-Ile-Gln-Cys)-NH₂,
(SEQ ID NO: 38)
4-Biphenylcarboxyl-c(Cys-Lys-Trp-Ile-Gln-Cys)-NH₂,
(SEQ ID NO: 38)
1-Naphthoyl-c(Cys-Lys-Trp-Ile-Gln-Cys)-NH₂,
(SEQ ID NO: 38)
2-Naphthoyl-c(Cys-Lys-Trp-Ile-Gln-Cys)-NH₂,
(SEQ ID NO: 38)
2-Indolecarboxyl-c(Cys-Lys-Trp-Ile-Gln-Cys)-NH₂,
(SEQ ID NO: 38)
p-Tolylacetyl-c(Cys-Lys-Trp-Ile-Gln-Cys)-NH₂,
(SEQ ID NO: 38)
trans-Cinnamoyl-c(Cys-Lys-Trp-Ile-Gln-Cys)-NH₂,
(SEQ ID NO: 38)
4-Chlorocinnamoyl-c(Cys-Lys-Trp-Ile-Gln-Cys)-NH₂,
(SEQ ID NO: 47)
c(Cys-Trp-Ile-Gln-Cys)-NH₂,
(SEQ ID NO: 48)
Ac-c(Cys-Lys-Trp-Ile-Gln-D-Cys)-NH₂,
(SEQ ID NO: 49)
Ac-c(Cys-Lys-Trp-Ile-D-Gln-Cys)-NH₂,
(SEQ ID NO: 50)
Ac-c(Cys-Lys-Trp-D-Ile-Gln-Cys)-NH₂,
(SEQ ID NO: 51)
Ac-c(Cys-Lys-D-Trp-Ile-Gln-Cys)-NH₂,
(SEQ ID NO: 52)
Ac-c(Cys-D-Lys-Trp-Ile-Gln-Cys)-NH₂,
(SEQ ID NO: 53)
Ac-c(D-Cys-Lys-Trp-Ile-Gln-Cys)-Nh₂,
(SEQ ID NO: 54)
(Des-amino-4-methyl)Phe-c(Cys-Lys-Trp-Ile-Gln-
Cys)-NH₂,
(SEQ ID NO: 38)
n-Hexanoyl-c(Cys-Lys-Trp-Ile-Gln-Cys)-NH₂,
(SEQ ID NO: 38)
Octanoyl-c(Cys-Lys-Trp-Ile-Gln-Cys)-NH₂,
(SEQ ID NO: 38)
Decanoyl-c(Cys-Lys-Trp-Ile-Gln-Cys)-NH₂,
(SEQ ID NO: 38)
Dodecanoyl-c(Cys-Lys-Trp-Ile-Gln-Cys)-NH₂,
(SEQ ID NO: 55)
Trp-c(Cys-Ile-Gln-Cys)-NH₂,
(SEQ ID NO: 56)
c(Cys-Ile-Gln-Cys)-NH₂,
(SEQ ID NO: 57)
c(Ac-Lys-Trp-Ile-Gln-hCys)-NH₂,
(SEQ ID NO: 58)
c(Gly-Lys-Trp-Ile-Gln-Asp)-NH₂,
(SEQ ID NO: 58)
c(Gly-Lys-Trp-Ile-Gln-Glu)-NH₂,
(SEQ ID NO: 42)
c((βAla-Lys-Trp-Ile-Gln-Glu)-NH₂,
(SEQ ID NO: 41)
c(Apn-Lys-Trp-Ile-Gln-Asp)-NH₂,
(SEQ ID NO: 42)
c(Apn-Lys-Trp-Ile-Gln-Glu)-NH₂,
(SEQ ID NO: 41)
c(Ahx-Lys-Trp-Ile-Gln-Asp)-NH₂,
(SEQ ID NO: 42)
c(Ahx-Lys-Trp-Ile-Gln-Glu)-NH₂,
(SEQ ID NO: 41)
c(Aha-Lys-Trp-Ile-Gln-Asp)-NH₂,
(SEQ ID NO: 42)
c(Aha-Lys-Trp-Ile-Gln-Glu)-NH₂,
(SEQ ID NO: 41)
c(Aoc-Lys-Trp-Ile-Gln-Asp)-NH₂,
(SEQ ID NO: 42)
c(Aoc-Lys-Trp-Ile-Gln-Glu)-NH₂,
(SEQ ID NO: 41)
c(Anc-Lys-Trp-Ile-Gln-Asp)-NH₂,
(SEQ ID NO: 42)
c(Anc-Lys-Trp-Ile-Gln-Glu)-NH₂,
(SEQ ID NO: 59)
Isobutyryl-c(Asp-Lys-Trp-Ile-Gln-Dap)-NH₂,
(SEQ ID NO: 59)
Isobutyryl-c(Asp-Lys-Trp-Ile-Gln-Orn)-NH₂,
(SEQ ID NO: 60)
Isobutyryl-c(Glu-Lys-Trp-Ile-Gln-Dap)-NH₂,
(SEQ ID NO: 60)
Isobutyryl-c(Glu-Lys-Trp-Ile-Gln-Dab)-NH₂,
(SEQ ID NO: 60)
Isobutyryl-c(Glu-Lys-Trp-Ile-Gln-Orn)-NH₂,
(SEQ ID NO: 60)
Isobutyryl-c(Glu-Lys-Trp-Ile-Gln-Lys)-NH₂,
(SEQ ID NO: 61)
Isobutyryl-c(Dap-Lys-Trp-Ile-Gln-Asp)-NH₂,
(SEQ ID NO: 61)
Isobutyryl-c(Dab-Lys-Trp-Ile-Gln-Asp)-NH₂,
(SEQ ID NO: 61)
Isobutyryl-c(Lys-Lys-Trp-Ile-Gln-Asp)-NH₂,
(SEQ ID NO: 62)
Isobutyryl-c(Dap-Lys-Trp-Ile-Gln-Glu)-NH₂,
(SEQ ID NO: 62)
Isobutyryl-c(Dab-Lys-Trp-Ile-Gln-Glu)-NH₂,
(SEQ ID NO: 62)
Isobutyryl-c(0rn-Lys-Trp-Ile-Gln-Glu)-NH₂,
(SEQ ID NO: 62)
Isobutyryl-c(Lys-Lys-Trp-Ile-Gln-Glu)-NH₂,
(SEQ ID NO: 63)
Ac-c(Asp-Lys-Trp-Ile-Gln-Dap)-NH₂,
(SEQ ID NO: 63)
Ac-c(Asp-Lys-Trp-Ile-Gln-Dab)-NH₂,
(SEQ ID NO: 63)
Ac-c(Asp-Lys-Trp-Ile-Gln-Orn)-NH₂,
(SEQ ID NO: 64)
Ac-c(Glu-Lys-Trp-Ile-Gln-Dap)-NH₂,
(SEQ ID NO: 64)
Ac-c(Glu-Lys-Trp-Ile-Gln-Dab)-NH₂,
(SEQ ID NO: 64)
Ac-c(Glu-Lys-Trp-Ile-Gln-Orn)-NH₂,
(SEQ ID NO: 64)
Ac-c(Glu-Lys-Trp-Ile-Gln-Lys)-NH₂,
(SEQ ID NO: 65)
Ac-c(Dap-Lys-Trp-Ile-Gln-Asp)-NH₂,
(SEQ ID NO: 65)
Ac-c(Dab-Lys-Trp-Ile-Gln-Asp)-NH₂,
(SEQ ID NO: 65)
Ac-c(0rn-Lys-Trp-Ile-Gln-Asp)-NH₂,
(SEQ ID NO: 65)
Ac-c(Lys-Lys-Trp-Ile-Gln-Asp)-NH₂,
(SEQ ID NO: 66)
Ac-c(Dap-Lys-Trp-Ile-Gln-Glu)-NH₂,
(SEQ ID NO: 66)
Ac-c(Dab-Lys-Trp-Ile-Gln-Glu)-NH₂,
(SEQ ID NO: 66)
Ac-c(0rn-Lys-Trp-Ile-Gln-Glu)-NH₂,
(SEQ ID NO: 66)
Ac-c(Lys-Lys-Trp-Ile-Gln-Glu)-NH₂,
(SEQ ID NO: 43)
c(Succinyl-Lys-Trp-Ile-Gln-Dap)-NH₂,
(SEQ ID NO: 43)
c(Succinyl-Lys-Trp-Ile-Gln-Dab)-NH₂,
(SEQ ID NO: 67)
c(Glutaryl-Lys-Trp-Ile-Gln-Dap)-NH₂,
(SEQ ID NO: 67)
c(Glutaryl-Lys-Trp-Ile-Gln-Dab)-NH₂,
(SEQ ID NO: 67)
c(Glutaryl-Lys-Trp-Ile-Gln-Orn)-NH₂,
(SEQ ID NO: 67)
c(Glutaryl-Lys-Trp-Ile-Gln-Lys)-NH₂,
(SEQ ID NO: 68)
c(Diglycolyl-Lys-Trp-Ile-Gln-Dap)-NH₂,
(SEQ ID NO: 68)
c(Diglycolyl-Lys-Trp-Ile-Gln-Dab)-NH₂,
(SEQ ID NO: 68)
c(Diglycolyl-Lys-Trp-Ile-Gln-Orn)-NH₂,
(SEQ ID NO: 68)
c(Diglycolyl-Lys-Trp-Ile-Gln-Lys)-NH₂,
(SEQ ID NO: 69)
c(Adipoyl-Lys-Trp-Ile-Gln-Dap)-NH₂,
(SEQ ID NO: 69)
c(Adipoyl-Lys-Trp-Ile-Gln-Dab)-NH₂,
(SEQ ID NO: 69)
c(Adipoyl-Lys-Trp-Ile-Gln-Orn)-NH₂,
or
(SEQ ID NO: 69)
c(Adipoyl-Lys-Trp-Ile-Gln-Lys)-NH2,
or a pharmaceutically acceptable salt thereof.

3. A compound according to claim 2 wherein said compound is
(SEQ ID NO: 38)
c(Cys-Lys-Trp-Ile-Gln-Cys)-NH₂,
(SEQ ID NO: 39)
Ac-Gln-c(Cys-Lys-Trp-Ile-Gln-Cys)-NH₂,
(SEQ ID NO: 38)
Isobutyryl-c(Cys-Lys-Trp-Ile-Gln-Cys)-NH₂,
(SEQ ID NO: 46)
c(Ac-Lys-Trp-Ile-Gln-Cys)-NH₂,
(SEQ ID NO: 38)
2-Indolecarboxyl-c(Cys-Lys-Trp-Ile-Gln-Cys)-NH₂,
(SEQ ID NO: 47)
c(Cys-Trp-Ile-Gln-Cys)-NH₂,
(SEQ ID NO: 38)
n-Hexanoyl-c(Cys-Lys-Trp-Ile-Gln-Cys)-NH₂,
(SEQ ID NO: 58)
c(Gly-Lys-Trp-Ile-Gln-Glu)-NH₂,
(SEQ ID NO: 41)
c(Ahx-Lys-Trp-Ile-Gln-Asp)-NH₂,
(SEQ ID NO: 42)
c(Ahx-Lys-Trp-Ile-Gln-Glu)-NH₂,
(SEQ ID NO: 42)
c(Aoc-Lys-Trp-Ile-Gln-Glu)-NH₂,
(SEQ ID NO: 59)
Isobutyryl-c(Asp-Lys-Trp-Ile-Gln-Dap)-NH₂,
(SEQ ID NO: 60)
Isobutyryl-c(Glu-Lys-Trp-Ile-Gln-Dab)-NH₂,
or
(SEQ ID NO: 62)
Isobutyryl-c(Dap-Lys-Trp-Ile-Gln-Glu)NH₂-,
or a pharmaceutically acceptable salt thereof.

4. A compound according to claim 3 wherein said compound is

| | |
|---|---|
| c(Cys-Trp-Ile-Gln-Cys)-NH₂, | (SEQ ID NO: 47) |
| or | |
| c(Gly-Lys-Trp-Ile-Gln-Glu)-NH₂, | (SEQ ID NO: 58) |

or a pharmaceutically acceptable salt thereof.

5. A method of preventing or inhibiting and indication associated with a chemokine-induced activity, comprising administering to a mammal afflicted with, or at risk of, the indication an amount of a chemokine peptide according to claim 1, or a pharmaceutically acceptable salt thereof, effective to prevent or inhibit said activity.

6. A method to inhibit the activity of more than one chemokine, comprising administering to a mammal in need thereof an effective amount of a chemokine peptide according to claim 1, or a pharmaceutically acceptable salt thereof.

7. A method to increase or enhance or chemokine-associated inflammatory response in a mammal, comprising administering to the mammal an amount of a chemokine peptide according to claim 1, or a pharmaceutically acceptable salt thereof, effective to increase or enhance said response.

8. A method of preventing or inhibiting an indication associated with hematopoietic cell recruitment, comprising administering to a mammal at risk of, or afflicted with, the indication of an effective amount of a chemokine peptide according to claim 1, or a pharmaceutically acceptable salt thereof.

9. A method of preventing or inhibiting an indication associated with histamine release from basophils or mast cells, comprising administering to a mammal at risk of, or afflicted with, the indication of an effective amount of a chemokine peptide according to claim 1, or a pharmaceutically acceptable salt thereof.

10. A method to modulate the chemokine-induced activity of hematopoietic cells at a preselected physiological site, comprising administering to a mammal a dosage form comprising an effective amount of a chemokine peptide according to claim 1, or a pharmaceutically acceptable salt thereof, wherein the dosage form is linked to a site targeting moiety.

11. A method to augment an immune response, comprising administering to a mammal an immunogenic moiety and an amount of a chemokine peptide according to claim 1, or a pharmaceutically acceptable salt thereof, wherein the amount is effective to augment the immune response of the mammal to the immunogenic moiety.

12. A therapeutic method to prevent or treat a vascular indication, comprising administering to a mammal in need of such therapy an effective amount of a chemokine peptide according to claim 1, or a pharmaceutically acceptable salt thereof, wherein the indication is coronary artery disease, myocardial infarction, unstable angina pectoris, atherosclerosis or vasculitis.

13. A therapeutic method to prevent or inhibit lentiviral infection or replication, comprising administering to a mammal in need of such therapy an effective amount of a chemokine peptide according to claim 1, or a pharmaceutically acceptable salt thereof.

14. The method of claim 13 wherein the lentivirus is HIV.

15. The method of claim 14 further comprising administering an antiviral agent before, during and/or after the administration of the peptide, a variant thereof, derivative thereof, the chemokine peptide according to claim 1, or a pharmaceutically acceptable salt thereof.

16. A therapeutic method to prevent or treat low bone mineral density, comprising administering to a mammal in need of such therapy an effective amount of a chemokine peptide according to claim 1, or a pharmaceutically acceptable salt thereof.

17. A method of inhibiting a parasitic infection in a vertebrate animal, comprising administering to a mammal in need of such therapy an effective amount of a chemokine Peptide according to claim 1, or a pharmaceutically acceptable salt thereof

18. The method of claim 17 wherein the animal is a human with malaria.

19. A therapeutic method to prevent or treat an autoimmune disease, comprising administering to a mammal in need of such therapy an effective amount of a chemokine peptide according to claim 1, or a pharmaceutically acceptable salt thereof.

20. A method of suppressing tumor growth in a vertebrate animal, comprising administering to said vertebrate an effective amount of a chemokine peptide according to claim 1, or a pharmaceutically acceptable salt thereof

21. A method for preventing or treating psoriasis in a mammal, comprising administering to said vertebrate an effective amount of a chemokine peptide according to claim 1, or a pharmaceutically acceptable salt thereof

22. A method to increase or enhance hematopoietic cell-associated activity at a tumor site, comprising administering an effective amount of a chemokine peptide according to claim 1, or a pharmaceutically acceptable salt thereof

23. A method enhance wound healing, comprising administering an effective amount of a chemokine peptide according to claim 1, or a pharmaceutically acceptable salt thereof.

24. A therapeutic method to prevent or treat asthma, comprising administering to a mammal in need of such therapy an effective amount of a chemokine peptide according to claim 1, or a pharmaceutically acceptable salt thereof.

25. A method of preventing or inhibiting an indication associated with elevated TNFα, comprising administering to a mammal afflicted with, or at risk of, the indication an effective amount of a chemokine peptide according to claim 1, or a pharmaceutically acceptable salt thereof.

26. A therapeutic method to prevent or treat organ transplant rejection, comprising administering to a mammal in need of such therapy an effective amount of a chemokine peptide according to claim 1, or a pharmaceutically acceptable salt thereof.

27. A therapeutic method to prevent or treat rheumatoid arthritis, comprising administering to a mammal in need of such therapy an effective amount of a chemokine peptide according to claim 1, or a pharmaceutically acceptable salt thereof.

28. A therapeutic method to prevent or treat allergy, comprising administering to a mammal in need of such therapy an effective amount of a chemokine peptide according to claim 1, or a pharmaceutically acceptable salt thereof.

29. Use of a chemokine peptide according to claim 1 for the manufacture of a medicament for the treatment of a pathological condition or symptom in a mammal which is associated with a chemokine-induced activity.

30. A peptide of the formula:
(SEQ ID NO: 70)
c(D-Cys-D-Gln-D-Ile-D-Trp-D-Lys-D-Gln-D-Lys-D-Pro-
D-Asp-D-Leu-D-Cys)-Doc-Aoc-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Aoc-OH,
(SEQ ID NO: 72)
c(D-Cys-D-Gln-D-Ile-D-Trp-D-Lys-D-Gln-D-Lys-D-Pro-
D-Asp-D-Leu-D-Cys)-Ile-Glu-Aoc-OH,
(SEQ ID NO: 73)
c(D-Cys-D-Gln-D-Ile-D-Trp-D-Lys-D-Gln-D-Lys-D-Pro-
D-Asp-D-Leu-D-Cys)-D-Val-D-Glu-Aoc-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Gaba-OH,
(SEQ ID NO: 71)
c(D-Cys-D-Gln-D-Ile-D-Trp-D-Lys-D-Gln-D-Lys-D-Pro-
D-Asp-D-Leu-D-Cys)-Ahx-OH,
(SEQ ID NO: 70)
c(D-Cys-D-Gln-D-Ile-D-Trp-D-Lys-D-Gln-D-Lys-D-Pro-
D-Asp-D-Leu-D-Cys)-Asp-Aoc-OH,
(SEQ ID NO: 70)
c(D-Cys-D-Gln-D-Ile-D-Trp-D-Lys-D-Gln-D-Lys-D-Pro-
D-Asp-D-Leu-D-Cys)-Glu-Aoc-OH,
(SEQ ID NO: 74)
c(D-Cys-D-Gln-D-Ile-D-Trp-D-Lys-D-Gln-D-Lys-D-Pro-
D-Asp-D-Leu-D-Cys)-D-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-D-Asp-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-Asp-Lys(Nε-octanoyl)-0H,
(SEQ ID NO: 75)
c(D-Cys-D-Gln-D-Ile-D-Trp-D-Lys-D-Gln-D-Lys-D-Pro-
D-Asp-D-Leu-D-Cys)-Lys(Nε-octanoyl)-OH,
(SEQ ID NO: 76)
c(D-Cys-D-Gln-D-Ile-D-Trp-D-Lys-D-Gln-D-Lys-D-Pro-
D-Asp-D-Leu-D-Cys)-D-Val-D-Glu-Lys(Nε-octanoyl)-
0H,
(SEQ ID NO: 75)
c(D-Cys-D-Gln-D-Ile-D-Trp-D-Lys-D-Gln-D-Lys-D-Pro-
D-Asp-D-Leu-D-Cys)-Doc-Lys(N/-octanoyl)-0H,
(SEQ ID NO: 77)
HEPA-c(D-Cys-D-Gln-D-Ile-D-Trp-D-Lys-D-Gln-D-Lys-
D-Pro-D-Asp-D-Leu-D-Cys)-NH₂,
(SEQ ID NO: 78)
c(D-Cys-D-Gln-D-Ile-D-Trp-D-Lys-D-Gln-D-Lys-D-Pro-
D-Asp-D-Leu-Lys(Nε-octanoyl)-D-Cys)-NH₂,
(SEQ ID NO: 79)
c(D-Cys-D-Gln-D-Ile-D-Trp-D-Lys-D-Gln-D-Lys-D-Pro-
D-Asp-Lys(Nε-octanoyl)-D-Leu-D-Cys)-OH,
(SEQ ID NO: 13)
Ac-c(Cys-Leu-Asp-Pro-Lys-Gln-Lys-Trp-A6c-Gln-
Cys)-NH₂,
(SEQ ID NO: 80)
Ac-c(Cys-A6c-Asp-Pro-Lys-Gln-Lys-Trp-Gln-Cys)-
NH₂,
(SEQ ID NO: 81)
Ac-c(Cys-Lys-Gln-Lys-Trp-Ile-Gln-Cys)-D-Pro-D-
Asp-D-Leu-NH₂,
(SEQ ID NO: 82)
Ac-c(Cys-Pro-Lys-Gln-Lys-Trp-Ile-Gln-Cys)-D-Asp-
D-Leu-NH₂,
(SEQ ID NO: 83)
c(D-Cys-D-Gln-D-Ile-D-Trp-D-Lys-D-Gln-D-Lys-D-Pro-
D-Asp(D-Ala-OH)-D-Leu-D-Cys)-NH₂,
(SEQ ID NO: 84)
Octanoyl-Dap-c(D-Cys-D-Gln-D-Ile-D-Trp-D-Lys-D-
Gln-D-Lys-D-Pro-D-Asp-D-Leu-D-Cys)-NH₂,
(SEQ ID NO: 111)
c(D-Cys-D-Gln-D-Ile-D-Trp-D-Lys-D-Gln-D-Lys-D-Pro-
D-Asp(1-hexanol)-D-Leu-D-Cys)-NH₂,
(SEQ ID NO: 85)
Ac-Lys(Ne-dodecanoyl)-c(Cys-Leu-Asp-Pro-Lys-Gln-
Lys-Trp-Ile-Gln-Cys)-NH₂,
(SEQ ID NO: 86)
BOC-Trp-Gly-α-aminoglutaramide,
(SEQ ID NO: 86)
BOC-Trp-Val-α-aminoglutaramide,
(SEQ ID NO: 86)
BOC-Trp-βAla-α-aminoglutaramide,
(SEQ ID NO: 87)
D-Lys(Nε-tetradecanoyl)-D-Asp-c(Cys-Leu-Asp-Pro-
Lys-Gln-Lys-Trp-Ile-Gln-Cys)-NH₂,
(SEQ ID NO: 88)
D-Lys-D-Asp-c(Cys-Leu-Asp-Pro-Lys-Gln-Lys-Trp-Ile-
Gln-Cys)-NH₂,
(SEQ ID NO: 87)
D-Lys(Nε-octanoyl)-D-Asp-c(Cys-Leu-Asp-Pro-Lys-
Gln-Lys-Trp-Ile-Gln-Cys)-NH₂,
(SEQ ID NO: 89)
Lys(Ne-dodecanoyl)-Ile-c(Cys-Leu-Asp-Pro-Lys-Gln-
Lys-Trp-Ile-Gln-Cys)-NH₂,
(SEQ ID NO: 89)
Lys(Nε-dodecanoyl)-Asp-c(Cys-Leu-Asp-Pro-Lys-Gln-
Lys-Trp-Ile-Gln-Cys)-NH₂,
(SEQ ID NO: 90)
BOC-Tcc-βAla-alpha-aminoglutaramide,
(SEQ ID NO: 91)
D-Lys(Nε-dodecanoyl)-Asp-c(Cys-Leu-Asp-Pro-Lys-
Gln-Lys-Trp-Ile-Gln-Cys)-NH₂,
(SEQ ID NO: 92)
Lys[Nε-[γ-Glu-(Nα-decanoyl)]}-c(Cys-Leu-Asp-Pro-Lys-
Gln-Lys-Trp-Ile-Gln-Cys)-NH₂,
(SEQ ID NO: 90)
BOC-Tic-βAla-α-aminoglutaramide,
(SEQ ID NO: 93)
3-indolepropionyl-L-valyl-α-aminoglutaramide,
(SEQ ID NO: 94)
N(3-indolylpropionyl)-βAla-α-aminoglutarimide,
(SEQ ID NO: 95)
Des-amino-Trp-βAla[ψ(CH₂NH)]-α-aminoglutarimide,
(SEQ ID NO: 94)
N-(3-indolepropyl)-βAla-α-aminoglutarimide,
(SEQ ID NO: 96)
N-(isopropoxycarbonyl)-Trp-βAla-α-
aminoglutarimide,
(SEQ ID NO: 97)
Trp-Gly-α-aminoglutaramide,
(SEQ ID NO: 97)
Trp-Val-α-aminoglutaramide,
(SEQ ID NO: 97)
Trp-βAla-α-aminoglutaramide,
(SEQ ID NO: 98)
c(D-Cys-D-Gln-D-Ile-D-Trp-D-Lys-D-Cys)-NH₂,
(SEQ ID NO: 99)
BOC-Trp-βhVal-α-aminoglutarimide,
(SEQ ID NO: 100)
Tryptaminylcarbonyl-βhVal-α-aminoglutarimide,
(SEQ ID NO: 101)
[aminoPeg₁₀-oxaglutaryl]₂-c(D-Cys-D-Gln-D-Ile-D-
Trp-D-Lys-D-Gln-D-Lys-D-Pro-D-Asp-D-Leu-D-Cys)-
NH₂,
(SEQ ID NO: 101)
[aminoPeg₁₀-oxaglutaryl]-c(D-Cys-D-Gln-D-Ile-D-
Trp-D-Lys-D-Gln-D-Lys-D-Pro-D-Asp-D-Leu-D-Cys)-
NH₂,
(SEQ ID NO: 102)
Trp-Aib-aminoglutarimide,
(SEQ ID NO: 103)
BOC-tryptophanolyl-carbonyl-Val-aminoglutarimide,
(SEQ ID NO: 98)
Ac-c(D-Cys-D-Gln-D-Ile-D-Trp-D-Lys-D-Cys)-NH₂,
(SEQ ID NO: 98)
nButyryl-c(D-Cys-D-Gln-D-Ile-D-Trp-D-Lys-D-Cys)-
NH₂,
(SEQ ID NO: 98)
Benzoyl-c(D-Cys-D-Gln-D-Ile-D-Trp-D-Lys-D-Cys)-
NH₂,
(SEQ ID NO: 98)
Isobutyryl-c(D-Cys-D-Gln-D-Ile-D-Trp-D-Lys-D-Cys)-
NH₂,
(SEQ ID NO: 104)
Amino-tryptophanolyl-carbonyl-Val-
aminoglutarimide,
(SEQ ID NO: 105)
c(βAla-D-Gln-D-Ile-D-Trp-D-Lys-D-Asp)-NH₂,
(SEQ ID NO: 105)
c(Gaba-D-Gln-D-Ile-D-Trp-D-Lys-D-Asp)-NH₂,
(SEQ ID NO: 105)
c(Apn-D-Gln-D-Ile-D-Trp-D-Lys-D-Asp)-NH₂,
(SEQ ID NO: 106)
Ac-c(D-Cys-D-Gln-D-Ile-D-Trp-D-Lys-D-Gln-D-Lys-D-
Pro-D-Asp-D-Leu-D-Cys)-NH₂,
(SEQ ID NO: 107)
c(βAla-D-Gln-D-Ile-D-Trp-D-Lys-D-Gln-D-Lys-D-Pro-
D-Asp-D-Leu-D-Asp)-NH₂,
(SEQ ID NO: 108)
Butanyl-c(D-Cys-D-Gln-D-Val-D-Trp-D-Lys-D-Cys)-
NH₂,
(SEQ ID NO: 109)
Isobutyryl-Asp-Lys-Trp-Ile-Gln-Lys-NH₂,
or
(SEQ ID NO: 110)
Isobutyryl-0m-Lys-Trp-Ile-Gln-Asp-NH₂,
or a pharmaceutically acceptable salt thereof.


SEQUENCE LISTING (112 sequences)

SEQ ID NO: 1 - protein, 11 aa, Artificial
  Chemokine analog
XLDPKQKWIQC

SEQ ID NO: 2 - protein, 11 aa, Artificial
  Chemokine analog
XCDPKQKWIQC

SEQ ID NO: 3 - protein, 11 aa, Artificial
  Chemokine analog
XDCPKQKWIQC

SEQ ID NO: 4 - protein, 11 aa, Artificial
  Chemokine analog
XDPKQCKWIQC

SEQ ID NO: 5 - protein, 11 aa, Artificial
  Chemokine analog
LDPKQKCWIQC

SEQ ID NO: 6 - protein, 11 aa, Artificial
  Chemokine analog
LDPKQKWCIQC

SEQ ID NO: 7 - protein, 6 aa, Artificial
  Chemokine analog
KWCIQC

SEQ ID NO: 8 - protein, 12 aa, Artificial
  Chemokine analog
CLDPKQKWIQCX

SEQ ID NO: 9 - protein, 13 aa, Artificial
  Chemokine analog
DCLDPKQKWIQCX

SEQ ID NO: 10 - protein, 11 aa, Artificial
  Chemokine analog
XDPCKQKWIQC

SEQ ID NO: 11 - protein, 11 aa, Artificial
  Chemokine analog
LDPKCQKWIQC

SEQ ID NO: 12 - protein, 11 aa, Artificial
  Chemokine analog
CLDPKQKWIXC

SEQ ID NO: 13 - protein, 11 aa, Artificial
  Chemokine analog
CLDPKQKWXQC

SEQ ID NO: 14 - protein, 11 aa, Artificial
  Chemokine analog
CLDPKQKXIQC

SEQ ID NO: 15 - protein, 11 aa, Artificial
  Chemokine analog
CLDPKQXWIQC

SEQ ID NO: 16 - protein, 11 aa, Artificial
  Chemokine analog
CLDPKXKWIQC

SEQ ID NO: 17 - protein, 11 aa, Artificial
  Chemokine analog
CLDPXQKWIQC

SEQ ID NO: 18 - protein, 11 aa, Artificial
  Chemokine analog
CLDXKQKWIQC

SEQ ID NO: 19 - protein, 11 aa, Artificial
  Chemokine analog
CLXPKQKWIQC

SEQ ID NO: 20 - protein, 11 aa, Artificial
  Chemokine analog
CXDPKQKWIQC

SEQ ID NO: 21 - protein, 12 aa, Artificial
  Chemokine analog
KCLDPKQKWIQC

SEQ ID NO: 22 - protein, 12 aa, Artificial
  Chemokine analog
XLDPKQKWIQCX

SEQ ID NO: 23 - protein, 11 aa, Artificial
  Chemokine analog
CLDPKQKWIQX

SEQ ID NO: 24 - protein, 11 aa, Artificial
  Chemokine analog
XLDPKQKWIQX

SEQ ID NO: 25 - protein, 11 aa, Artificial
  Chemokine analog
XLDPKQKWIQC

SEQ ID NO: 26 - protein, 10 aa, Artificial
  Chemokine analog
DPCKQKWIQC

SEQ ID NO: 27 - protein, 10 aa, Artificial
  Chemokine analog
DPKCQKWIQC

SEQ ID NO: 28 - protein, 10 aa, Artificial
  Chemokine analog
CDPKQKWIQC

SEQ ID NO: 29 - protein, 9 aa, Artificial
  Chemokine analog
PCKQKWIQC

SEQ ID NO: 30 - protein, 9 aa, Artificial
  Chemokine analog
PKCQKWIQC

SEQ ID NO: 31 - protein, 9 aa, Artificial
  Chemokine analog
PKQCKWIQC

SEQ ID NO: 32 - protein, 9 aa, Artificial
  Chemokine analog
CPKQKWIQC

SEQ ID NO: 33 - protein, 8 aa, Artificial
  Chemokine analog
KCQKWIQC

SEQ ID NO: 34 - protein, 8 aa, Artificial
  Chemokine analog
KQCKWIQC

SEQ ID NO: 35 - protein, 8 aa, Artificial
  Chemokine analog
CKQKWIQC

SEQ ID NO: 36 - protein, 7 aa, Artificial
  Chemokine analog
CQKWIQC

SEQ ID NO: 37 - protein, 6 aa, Artificial
  Chemokine analog
KCWIQC

SEQ ID NO: 38 - protein, 6 aa, Artificial
  Chemokine analog
CKWIQC

SEQ ID NO: 39 - protein, 7 aa, Artificial
  Chemokine analog
XCKWIQC

SEQ ID NO: 40 - protein, 5 aa, Artificial
  Chemokine analog
XKWQD

SEQ ID NO: 41 - protein, 6 aa, Artificial
  Chemokine analog
XKWIQD

SEQ ID NO: 42 - protein, 6 aa, Artificial
  Chemokine analog
XKWIQE

SEQ ID NO: 43 - protein, 5 aa, Artificial
  Chemokine analog
KWIQX

SEQ ID NO: 44 - protein, 5 aa, Artificial
  Chemokine analog
KWIQK

SEQ ID NO: 45 - protein, 6 aa, Artificial
  Chemokine analog
DKWIQK

SEQ ID NO: 46 - protein, 5 aa, Artificial
  Chemokine analog
KWIQC

SEQ ID NO: 47 - protein, 5 aa, Artificial
  Chemokine analog
CWIQC

SEQ ID NO: 48 - protein, 6 aa, Artificial
  Chemokine analog
CKWIQX

SEQ ID NO: 49 - protein, 6 aa, Artificial
  Chemokine analog
CKWIXC

SEQ ID NO: 50 - protein, 6 aa, Artificial
  Chemokine analog
CKWXQC

SEQ ID NO: 51 - protein, 6 aa, Artificial
  Chemokine analog
CKXIQC

SEQ ID NO: 52 - protein, 6 aa, Artificial
  Chemokine analog
CXWIQC

SEQ ID NO: 53 - protein, 6 aa, Artificial
  Chemokine analog
XKWIQC

SEQ ID NO: 54 - protein, 7 aa, Artificial
  Chemokine analog
FCKWIQC

SEQ ID NO: 55 - protein, 5 aa, Artificial
  Chemokine analog
WCIQC

SEQ ID NO: 56 - protein, 4 aa, Artificial
  Chemokine analog
CIQC

SEQ ID NO: 57 - protein, 5 aa, Artificial
  Chemokine analog
KWIQX

SEQ ID NO: 58 - protein, 6 aa, Artificial
  Chemokine analog
GKWIQX

SEQ ID NO: 59 - protein, 6 aa, Artificial
  Chemokine analog
DKWIQX

SEQ ID NO: 60 - protein, 6 aa, Artificial
  Chemokine analog
EKWIQX

SEQ ID NO: 61 - protein, 6 aa, Artificial
  Chemokine analog
XKWIQD

SEQ ID NO: 62 - protein, 6 aa, Artificial
  Chemokine analog
XKWIQE

SEQ ID NO: 63 - protein, 6 aa, Artificial
  Chemokine analog
DKWIQX

SEQ ID NO: 64 - protein, 6 aa, Artificial
  Chemokine analog
EKWIQX

SEQ ID NO: 65 - protein, 6 aa, Artificial
  Chemokine analog
XKWIQD

SEQ ID NO: 66 - protein, 6 aa, Artificial
  Chemokine analog
XKWIQE

SEQ ID NO: 67 - protein, 5 aa, Artificial
  Chemokine analog
KWIQX

SEQ ID NO: 68 - protein, 5 aa, Artificial
  Chemokine analog
KWIQX

SEQ ID NO: 69 - protein, 5 aa, Artificial
  Chemokine analog
KWIQX

SEQ ID NO: 70 - protein, 13 aa, Artificial
  Chemokine analog
XXXXXXXXXXXXX

SEQ ID NO: 71 - protein, 12 aa, Artificial
  Chemokine analog
XXXXXXXXXXXX

SEQ ID NO: 72 - protein, 14 aa, Artificial
  Chemokine analog
XXXXXXXXXXXIEX

SEQ ID NO: 73 - protein, 14 aa, Artificial
  Chemokine analog
XXXXXXXXXXXXXX

SEQ ID NO: 74 - protein, 12 aa, Artificial
  Chemokine analog
XXXXXXXXXXXX

SEQ ID NO: 75 - protein, 13 aa, Artificial
  Chemokine analog
XXXXXXXXXXXXX

SEQ ID NO: 76 - protein, 14 aa, Artificial
  Chemokine analog
XXXXXXXXXXXXXX

SEQ ID NO: 77 - protein, 11 aa, Artificial
  Chemokine analog
XXXXXXXXXXX

SEQ ID NO: 78 - protein, 12 aa, Artificial
  Chemokine analog
XXXXXXXXXXXX

SEQ ID NO: 79 - protein, 12 aa, Artificial
  Chemokine analog
XXXXXXXXXXXX

SEQ ID NO: 80 - protein, 10 aa, Artificial
  Chemokine analog
CXDPKQKWQC

SEQ ID NO: 81 - protein, 11 aa, Artificial
  Chemokine analog
CKQKWIQCXXX

SEQ ID NO: 82 - protein, 11 aa, Artificial
  Chemokine analog
CPKQKWIQCXX

SEQ ID NO: 83 - protein, 12 aa, Artificial
  Chemokine analog
XXXXXXXXXXXX

SEQ ID NO: 84 - protein, 12 aa, Artificial
  Chemokine analog
XXXXXXXXXXXX

SEQ ID NO: 85 - protein, 12 aa, Artificial
  Chemokine analog
XCLDPKQKWIQC

SEQ ID NO: 86 - protein, 2 aa, Artificial
  Chemokine analog
WX

SEQ ID NO: 87 - protein, 13 aa, Artificial
  Chemokine analog
XXCLDPKQKWIQC

SEQ ID NO: 88 - protein, 13 aa, Artificial
  Chemokine analog
XXCLDPKQKWIQC

SEQ ID NO: 89 - protein, 13 aa, Artificial
  Chemokine analog
XXCLDPKQKWIQC

SEQ ID NO: 90 - protein, 2 aa, Artificial
  Chemokine analog
XX

SEQ ID NO: 91 - protein, 13 aa, Artificial
  Chemokine analog
XDCLDPKQKWIQC

SEQ ID NO: 92 - protein, 12 aa, Artificial
  Chemokine analog
XCLDPKQKWIQC

SEQ ID NO: 93 - protein, 1 aa, Artificial
  Chemokine analog
X

SEQ ID NO: 94 - protein, 1 aa, Artificial
  Chemokine analog
X

SEQ ID NO: 95 - protein, 2 aa, Artificial
  Chemokine analog
WX

SEQ ID NO: 96 - protein, 2 aa, Artificial
  Chemokine analog
WX

SEQ ID NO: 97 - protein, 2 aa, Artificial
  Chemokine analog
WX

SEQ ID NO: 98 - protein, 6 aa, Artificial
  Chemokine analog
XXXXXX

SEQ ID NO: 99 - protein, 2 aa, Artificial
  Chemokine analog
WX

SEQ ID NO: 100 - protein, 1 aa, Artificial
  Chemokine analog
X

SEQ ID NO: 101 - protein, 11 aa, Artificial
  Chemokine analog
XXXXXXXXXXX

SEQ ID NO: 102 - protein, 2 aa, Artificial
  Chemokine analog
WX

SEQ ID NO: 103 - protein, 1 aa, Artificial
  Chemokine analog
X

SEQ ID NO: 104 - protein, 1 aa, Artificial
  Chemokine analog
X

SEQ ID NO: 105 - protein, 6 aa, Artificial
  Chemokine analog
XXXXXX

SEQ ID NO: 106 - protein, 11 aa, Artificial
  Chemokine analog
XXXXXXXXXXX

SEQ ID NO: 107 - protein, 11 aa, Artificial
  Chemokine analog
XXXXXXXXXXX

SEQ ID NO: 108 - protein, 6 aa, Artificial
  Chemokine analog
XXXXXX

SEQ ID NO: 109 - protein, 6 aa, Artificial
  Chemokine analog
DKWIQK

SEQ ID NO: 110 - protein, 6 aa, Artificial
  Chemokine analog
XKWIQD

SEQ ID NO: 111 - protein, 11 aa, Artificial
  Chemokine analog
XXXXXXXXXXX

SEQ ID NO: 112 - protein, 6 aa, Artificial
  Chemokine analog
CKWIQX